(19) United States
(12) Patent Application Publication
(10) Pub. No.: US 2014/0248263 A1
(43) Pub. Date: Sep. 4, 2014

(54) BISPHENOL COMPOUNDS AND METHODS FOR THEIR USE

(71) Applicants: Raymond John Andersen, Vancouver (CA); Javier Garcia Fernandez, Asturias (ES); Marianne Dorothy Sadar, West Vancouver (CA); Nasrin Mawji, Burnaby (CA); Carmen Adriana Banuelos, Richmond (CA); Jun Wang, Surrey (CA)

(72) Inventors: Raymond John Andersen, Vancouver (CA); Javier Garcia Fernandez, Asturias (ES); Marianne Dorothy Sadar, West Vancouver (CA); Nasrin Mawji, Burnaby (CA); Carmen Adriana Banuelos, Richmond (CA); Jun Wang, Surrey (CA)

(73) Assignees: THE UNIVERSITY OF BRITISH COLUMBIA, Vancouver, BC (CA); BRITISH COLUMBIA CANCER AGENCY BRANCH, Vancouver, BC (CA)

(21) Appl. No.: 14/110,615

(22) PCT Filed: Apr. 6, 2012
(86) PCT No.: PCT/US2012/032584

§ 371 Date: May 23, 2014

Related U.S. Application Data
(60) Provisional application No. 61/473,676, filed on Apr. 8, 2011.

(51) Int. Cl. A61K 31/336; A61K 45/06; A61K 31/09; C07C 43/215; C07D 303/14; C07C 43/23

(52) U.S. Cl. CPC A61K 31/336; C07D 303/14; C07C 43/23; A61K 31/09; C07C 43/215; A61K 45/06
USPC 424/133.1; 549/559; 514/475; 568/609; 514/720; 514/719

(57) ABSTRACT

Compounds having a structure of Formula I: wherein G, a, Q, L², R¹, R², R³, R⁴, R⁵ and R⁶ are as defined herein are provided. Uses of such compounds for treatment of various indications, including prostate cancer as well as methods of treatment involving such compounds are also provided.

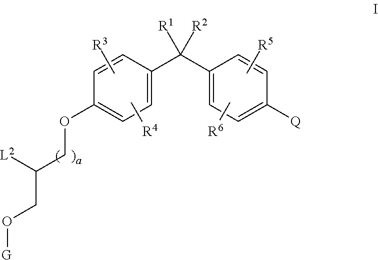

### CROSS-REFERENCE TO RELATED APPLICATION

This application claims the benefit under 35 U.S.C. §119(e) of U.S. Provisional Patent Application No. 61/473,676, filed Apr. 8, 2011, which application is incorporated herein by reference in its entirety.

### STATEMENT OF GOVERNMENT INTEREST

This invention was made in part with government support under Grant No. R01 CA105304 awarded by the National Cancer Institute. The United States Government has certain rights in this invention.

### FIELD OF INVENTION

This invention relates to therapeutics for treatment of various indications, including various cancers. In particular the invention relates to bisphenol compounds and use of the same for treatment of cancers, such as all stages of prostate cancer, including all androgen dependent, androgen-sensitive and castration-resistant prostate cancers.

### BACKGROUND OF THE INVENTION

Androgens mediate their effects through the androgen receptor (AR). Androgens play a role in a wide range of developmental and physiological responses and are involved in male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration (androgen ablation).

Androgens also play a role in female cancers. One example is ovarian cancer where elevated levels of androgens are associated with an increased risk of developing ovarian cancer (K. J. Helzlsouer, A. J. Alberg, G. B. Gordon, C. Longcope, T. L. Bush, S. C. Hoffman & G. W. Comstock, JAMA 274, 1926-1930 (1995); R. J. Edmondson, J. M. Monaghan & B. R. Davies, Br J Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit. Rev Oncol Hematol 25, 1-9 (1997)), whereas estrogen receptor-alpha (ERa) and the progesterone receptor are detected in less than 50% of ovarian tumors.

The only effective treatment available for advanced prostate cancer is the withdrawal of androgens which are essential for the survival of prostate epithelial cells. Androgen ablation therapy causes a temporary reduction in tumor burden concomitant with a decrease in serum prostate-specific antigen (PSA). Unfortunately prostate cancer can eventually grow again in the absence of testicular androgens (castration-resistant disease) (Huber et al 1987 Scand J. Urol Nephrol. 104, 33-39). Castration-resistant prostate cancer is biochemically characterized before the onset of symptoms by a rising titre of serum PSA (Miller et al 1992 J. Urol. 147, 956-961). Once the disease becomes castration-resistant most patients succumb to their disease within two years.

The AR has distinct functional domains that include the carboxy-terminal ligand-binding domain (LBD), a DNA-binding domain (DBD) comprising two zinc finger motifs, and an N-terminus domain (NTD) that contains one or more transcriptional activation domains. Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (IL-6) and by various growth factors (Culig et at 1994 Cancer Res. 54, 5474-5478; Nazareth et al 1996 J. Biol. Chem. 271, 19900-19907; Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The AR may be activated in the absence of testicular androgens by alternative signal transduction pathways in castration resistant disease, which is consistent with the finding that nuclear AR protein is present in secondary prostate cancer tumors (Kim et al 2002 Am. J. Pathol. 160, 219-226; and van der Kwast et al 1991 Inter. J. Cancer 48, 189-193).

Available inhibitors of the AR include nonsteroidal antiandrogens such as bicalutamide (Casodex™), nilutamide, flutamide, investigational drugs MDV3100 and ARN-509, and the steroidal antiandrogen, cyproterone acetate. These antiandrogens target the LBD of the AR and predominantly fail presumably due to poor affinity and mutations that lead to activation of the AR by these same antiandrogens (Taplin, M. E., Bubley, G. J., Kom Y. J., Small E. J., Uptonm M., Rajeshkumarm B., Balkm S. P., Cancer Res., 59, 2511-2515 (1999)). These antiandrogens would also have no effect on the recently discovered AR splice variants that lack the ligand-binding domain (LBD) to result in a constitutively active receptor which promotes progression of androgen-independent prostate cancer (Dehm S M, Schmidt L J, Heemers H V, Vessella R L, Tindall D J., Cancer Res 68, 5469-77, 2008; Guo Z, Yang X, Sun F, Jiang R, Linn D E, Chen H, Chen H, Kong X, Melamed J, Tepper C G, Kung H J, Brodie A M, Edwards J, Qiu Y., Cancer Res. 69, 2305-13, 2009; Hu et al 2009 Cancer Res. 69, 16-22; Sun et al 2010 J Clin Invest. 2010 120, 2715-30).

Conventional therapy has concentrated on androgen-dependent activation of the AR through its C-terminal domain. Recent studies developing antagonists to the AR have concentrated on the C-terminus and specifically: 1) the allosteric pocket and AF-2 activity (Estébanez-Perpiñá et al 2007, PNAS104, 16074-16079); 2) in silico “drug repurposing” procedure for identification of nonsteroidal antagonists (Bisson et al 2007, PNAS104, 11927-11932); and coactivator or corepressor interactions (Chang et al 2005, Mol Endocrinology 19, 2478-2490; Hur et al 2004, PLoS Biol 2, E274; Estébanez-Perpiñá et al 2005, JBC 280, 8060-8068; He et al 2004, Mol Cell 16, 425-438). The AR-NTD is also a target for drug development (e.g. WO 2000/001813), since the NTD contains Activation-Function-1 (AF1) which is the essential region required for AR transcriptional activity (Jenster et al 1991. Mol. Endocrinol. 5, 1396-404). The AR-NTD importantly plays a role in activation of the AR in the absence of androgens (Sadar, M. D. 1999 J. Biol. Chem. 274, 7777-7783; Sadar M D et al 1999 Endocr Relat Cancer. 6, 487-502; Ueda et al 2002 J. Biol. Chem. 277, 7076-7085; Ueda 2002 J. Biol. Chem. 277, 38087-38094; Blaszczyk et al 2004 Clin Cancer Res. 10, 1860-9; Dehm et al 2006 J Biol. Chem. 28, 27882-93; Gregory et al 2004 J Biol. Chem. 279, 7119-30). The AR-NTD is important in hormonal progression of prostate cancer as shown by application of decoy molecules (Quayle et al 2007, Proc Natl Acad Sci USA. 104, 1331-1336).

While the crystal structure has been resolved for the AR C-terminus LBD, this has not been the case for the NTD due to its high flexibility and intrinsic disorder in solution (Reid et al 2002 J. Biol. Chem. 277, 20079-20086) thereby hampering virtual docking drug discovery approaches.

Recent advances in the development of compounds that modulate AR include the bis-phenol compounds disclosed in published PCT WO 2010/000066 to the British Columbia Cancer Agency Branch and The University of British Columbia. While such compounds appear promising, there remains a need in the art for additional and/or improved compounds that modulate the AR, and which provide treatment for conditions that benefit from such modulation

### BRIEF SUMMARY

The compounds described herein may be used for in vivo or in vitro research uses (i.e., non-clinical) to investigate the mechanisms of orphan and nuclear receptors (including steroid receptors such as the androgen receptor). Furthermore, these compounds may be used individually or as part of a kit for in vivo or in vitro research to investigate signal transduction pathways and/or the activation of orphan and nuclear receptors using recombinant proteins, cells maintained in culture, and/or animal models.

This invention is also based in part on the surprising discovery that the compounds described herein, may also be used to modulate AR activity either in vivo or in vitro for both research and therapeutic uses. The compounds may be used in an effective amount so that androgen receptor activity may be modulated. The AR may be mammalian. Alternatively, the androgen receptor may be human. In particular, the compounds may be used to inhibit the AR. The compounds modulatory activity may be used in either an in vivo or an in vitro model for the study of at least one of the following indications: prostate cancer, breast cancer, ovarian cancer, salivary gland carcinoma, endometrial cancer, hair loss, acne, hirsutism, ovarian cysts, polycystic ovary disease, precocious puberty (testoxicosis), spinal and bulbar muscular atrophy (SBMA, Kennedy's disease), and age-related macular degeneration. Furthermore, the compounds modulatory activity may be used for the treatment of at least one of the following indications: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. The indication for treatment may be prostate cancer. The prostate cancer may be castration-resistant prostate cancer. The prostate cancer may be androgen-dependent prostate cancer. In other examples the indication is Kennedy's disease.

In accordance with one embodiment, there is provided a compound having a structure of Formula I:

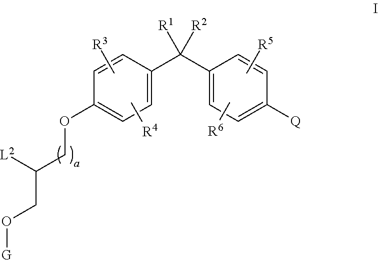

or a pharmaceutically acceptable salt thereof, wherein G, a, Q, L², R¹, R², R³, R⁴, R⁵ and R⁶ are as defined herein.

In other certain embodiments, the present disclosure provides the use of a compound of Formula I, for modulating androgen receptor (AR) activity. Methods for modulating AR, as well as pharmaceutical compositions comprising a compound of Formula I and a pharmaceutically acceptable excipient are also provided.

The present disclosure also provides combination therapy treatments for any of the diseases states disclosed herein. The disclosed thereapies include use of a pharmaceutical composition comprising a compound of Formula I, an additional therapeutic agent and pharmaceutically acceptable excipient. Methods and compositions related to the same are also provided.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE SEVERAL VIEWS OF THE DRAWINGS

In the drawings, the sizes and relative positions of elements in the drawings are not necessarily drawn to scale. For example, the various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been selected solely for ease of recognition in the drawings.

FIG. 1 shows LNCaP and PC3 in vitro data for a representative compound.

FIG. 2 presents LNCaP and PC3 in vitro data for a representative compound.

FIG. 3 is a graph showing LNCaP and PC3 in vitro data for a representative compound.

FIG. 4 illustrates in vivo dose response for a representative compound.

FIG. 5 shows dose response of a representative compound and a comparative compound.

FIG. 6 presents dose response data for a representative compound and a comparative compound.

### DETAILED DESCRIPTION

As used herein, the phrase “Cₓ-C_{y} alkyl” is used as it is normally understood to a person of skill in the art and often refers to a chemical entity that has a carbon skeleton or main carbon chain comprising a number from x to y (with all individual integers within the range included, including integers x and y) of carbon atoms. For example a “C₁-C₁₀ alkyl” is a chemical entity that has 1, 2, 3, 4, 5, 6, 7, 8, 9 or 10 carbon atom(s) in its carbon skeleton or main chain and a “C₁-C₂₀ alkyl” is a chemical entity that has 1, 2, 3, 4, 5, 6, 7, 8, 9, 10, 11, 12, 13, 14, 15, 16, 17, 18, 19 or 20 carbon atom(s) in its carbon skeleton or main chain.

As used herein, the term “cyclic C_{X}-C_{y} alkyl” is used as it is normally understood to a person of skill in the art and often refers to a compound or a chemical entity in which at least a portion of the carbon skeleton or main chain of the chemical entity is bonded in such a way so as to form a ‘loop’, circle or ring of atoms that are bonded together. The atoms do not have to all be directly bonded to each other, but rather may be directly bonded to as few as two other atoms in the ‘loop’. Non-limiting examples of cyclic alkyls include benzene, toluene, cyclohexane, cyclopentane, bisphenol and 1-chloro-3-ethylcyclohexane.

As used herein, the term “branched” is used as it is normally understood to a person of skill in the art and often refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl are tert-butyl and isopropyl.

As used herein, the term “unbranched” is used as it is normally understood to a person of skill in the art and often refers to a chemical entity that comprises a skeleton or main chain that does not split off into more than one contiguous chain. Non-limiting examples of unbranched alkyls are methyl, ethyl, n-propyl, and n-butyl.

As used herein, the term “substituted” is used as it is normally understood to a person of skill in the art and often refers to a chemical entity that has one chemical group replaced with a different chemical group which may contain one or more heteroatoms. Unless otherwise specified, a substituted alkyl is an alkyl in which one or more hydrogen atom(s) is/are replaced with one or more atom(s) that is/are not hydrogen(s). For example, chloromethyl is a non-limiting example of a substituted alkyl, more particularly an example of a substituted methyl. Aminoethyl is another non-limiting example of a substituted alkyl, more particularly it is a substituted ethyl. Unless specifically stated otherwise, all groups described herein are “optionally substituted”, meaning they may be substituted or unsubstituted.

As used herein, the term “unsubstituted” is used as it is normally understood to a person of skill in the art and often refers to a chemical entity that is a hydrocarbon and/or does not contain a heteroatom. Non-limiting examples of unsubstituted alkyls include methyl, ethyl, tert-butyl, and pentyl.

As used herein, the term “saturated” when referring to a chemical entity is used as it is normally understood to a person of skill in the art and often refers to a chemical entity that comprises only single bonds. Non-limiting examples of saturated chemical entities include ethane, tert-butyl, and N⁺H₃.

Unless specifically stated otherwise, C₁-C₂₀ alkyl may include, for example, and without limitation, saturated C₁-C₂₀ alkyl, C₂-C₂₀ alkenyl and C₂-C₂₀ alkynyl. Non-limiting examples of saturated C₁-C₂₀ alkyl may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, 1-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl, t-decyl and the like. Non-limiting examples of C₂-C₂₀ alkenyl may include vinyl, allyl, isopropenyl, 1-prop ene-2-yl, 1-butene-1-yl, 1-butene-2-yl, 1-butene-3-yl, 2-butene-1-yl, 2-butene-2-yl, octenyl, decenyl and the like. Non-limiting examples of C₂-C₂₀ alkynyl may include ethynyl, propynyl, butynyl, pentynyl, hexynyl, heptynyl, octynyl, nonynyl, decynyl and the like. Saturated C₁-C₂₀ alkyl, C₂-C₂₀ alkenyl or C₂-C₂₀ alkynyl may be, for example, and without limitation, interrupted by one or more heteroatoms which are independently nitrogen, sulfur or oxygen.

Unless specifically stated otherwise, C₁-C₁₀ alkyl may include, for example, and without limitation, saturated C₁-C₁₀ alkyl, C₂-C₁₀ alkenyl and C₂-C₁₀ alkynyl. Non-limiting examples of saturated C₁-C₁₀ alkyl may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, 1-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl and t-decyl. Non-limiting examples of C₂-C₁₀ alkenyl may include vinyl, allyl, isopropenyl, 1-prop ene-2-yl, 1-butene-1-yl, 1-butene-2-yl, 1-butene-3-yl, 2-butene-1-yl, 2-butene-2-yl, octenyl and decenyl. Non-limiting examples of C₂-C₁₀ alkynyl may include ethynyl, propynyl, butynyl, pentynyl, hexynyl, heptynyl, octynyl, nonynyl and decynyl. Saturated C₁-C₁₀ alkyl, C₂-C₁₀ alkenyl or C₂-C₁₀ alkynyl may be, for example, and without limitation, interrupted by one or more heteroatoms which are independently nitrogen, sulfur or oxygen.

As used herein, cyclic C₃-C₁₀ alkyl may include, for example, and without limitation, saturated C₃-C₁₀ cycloalkyl, C₃-C₁₀ cycloalkenyl, C₃-C₁₀ cycloalkynyl, C₆₋₁₀ aryl, C₆₋₉ aryl-C₁₋₄ alkyl, C₆₋₈ aryl-C₂₋₄ alkenyl, C₆₋₈ aryl-C₂₋₄ alkynyl, a 4- to 10-membered non-aromatic heterocyclic group containing one or more heteroatoms which are independently nitrogen, sulfur or oxygen, and a 5- to 10-membered aromatic heterocyclic group containing one or more heteroatoms which are independently nitrogen, sulfur or oxygen. Non-limiting examples of the saturated C₃-C₁₀ cycloalkyl group may include cyclopropanyl, cyclobutanyl, cyclopentanyl, cyclohexanyl, cycloheptanyl, cyclooctanyl, cyclononanyl and cyclodecanyl. Non-limiting examples of the C₃-C₁₀ cycloalkenyl group may include cyclopropenyl, cyclobutenyl, cyclopentenyl, cyclohexenyl, cycloheptenyl, cyclooctenyl, cyclononanenyl and cyclodecanenyl. Non-limiting examples of the C₆-C₁₀ aryl group may include phenyl (Ph), pentalenyl, indenyl, naphthyl, and azulenyl. The C₆₋₉ aryl-C₁₋₄ alkyl group may be, for example, and without limitation, a C₁₋₄ alkyl group as defined anywhere above having a C₆₋₉ aryl group as defined anywhere above as a substituent. The C₆₋₈ aryl-C₂₋₄ alkenyl group may be, for example, and without limitation, a C₂₋₄ alkenyl as defined anywhere above having a C₆₋₈ aryl group as defined anywhere above as a substituent. The C₆₋₈ aryl-C₂₋₄ alkynyl group may be, for example, and without limitation, a C₂₋₄ alkynyl group as defined anywhere above having a C₆₋₈ aryl group as defined anywhere above as a substituent. Non-limiting examples of the 4- to 10-membered non-aromatic heterocyclic group containing one or more heteroatoms which are independently nitrogen, sulfur or oxygen may include pyrrolidinyl, pyrrolinyl, piperidinyl, piperazinyl, imidazolinyl, pyrazolidinyl, imidazolydinyl, morpholinyl, tetrahydropyranyl, azetidinyl, oxetanyl, oxathiolanyl, phthalimide and succinimide. Non-limiting examples of the 5- to 10-membered aromatic heterocyclic group containing one or more heteroatoms which are independently nitrogen, sulfur or oxygen may include pyrrolyl, pyridinyl, pyridazinyl, pyrimidinyl, pirazinyl, imidazolyl, thiazolyl and oxazolyl.

Non-limiting examples of one to ten carbon substituted or unsubstituted acyl include acetyl, propionyl, butanoyl and pentanoyl. Non-limiting examples of C₁-C₁₀ alkoxy include methoxy, ethoxy, propoxy and butoxy.

As used herein, the symbol “ [symbol] ” (hereinafter may be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “XY” is bonded to another chemical entity via the point of attachment bond. Furthermore, the specific point of attachment to the non-depicted chemical entity may be specified by inference. For example The compound CH₃—R³, wherein R³ is H or “ [symbol] ” infers that when R³ is “XY”, the point of attachment bond is the same bond as the bond by which R³ is depicted as being bonded to CH₃.

“Halo” refers to fluoro (F), chloro (Cl), bromo (Br) or iodo (I). Radioisotopes are included witin the definition of halo. Accordingly, compounds comprising fluoro, chloro, bromo or iodo may also comprise radioisotopes of the same.

As noted above, the present disclosure provides a compound having a structure of Formula I:

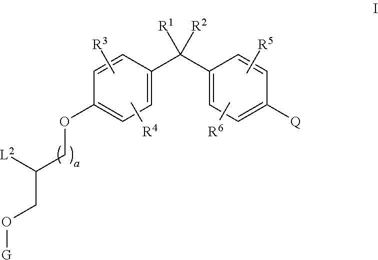

or a pharmaceutically acceptable salt thereof, wherein:

G is a linear, branched or non-aromatic cyclic, substituted or unsubstituted, saturated or unsaturated C₁-C₂₀ alkyl, wherein one or more carbon atoms of the C₁-C₂₀ alkyl may optionally be replaced with an oxygen atom;

a is 0 or 1;

R¹ and R² are each independently H or linear or branched, substituted or unsubstituted, saturated or unsaturated C₁-C₁₀ alkyl, or R¹ and R² together may form a substituted or unsubstituted, saturated or unsaturated cyclic C₃-C₁₀ alkyl;

R³, R⁴, R⁵ and R⁶ are each independently H, halo or linear or branched, substituted or unsubstituted, saturated or unsaturated C₁-C₁₀ alkyl;

Q is

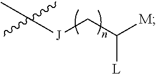

J is G¹, O, CH₂, CHG¹, CG¹₂, S, NH, NG¹, SO, SO₂, or NR;

M is H, OH, F, Cl, Br, CH₂OH, CH₂F, CH₂Cl, CHCl₂, CCl₃, CH₂Br, CHBr₂ or CBr₃;

L is H, A-D or —CH₂-A-D;

A is O, S, NH, NG¹, N⁺H₂, or N⁺HG¹;

D is H, G¹, R, or a moiety from TABLE 1;

R is C₁-C₁₀ acyl;

n is 0, 1, 2, 3, 4, 5, 6, 7 or 8;

L² is H or A²-D²;

A² is O, S, SO, SO₂, NH, NG¹, N⁺H₂ or N⁺HG¹;

D² is H, G¹, R⁷, or a moiety from TABLE 1;

G¹ is a linear or branched, aromatic or non-aromatic cyclic, substituted or unsubstituted, saturated or unsaturated C₁-C₁₀ alkyl;

provided that M is not CH₂Cl when G is isopropyl and L is not H when G is a saturated C₁-C₂₀ alkyl, wherein one or more carbon atoms of the saturated C₁-C₂₀ alkyl have been replaced with an oxygen atom.

In certain embodiments, the optional substituents for any of the C₁-C₂₀ alkyl, C₁-C₁₀ alkyl and cyclic C₃-C₁₀ alkyl moieties are each independently oxo, OR⁸, COOH, R⁹, OH, OR⁹, F, Cl, Br, I, NH₂, NHR⁹, N(R⁹)₂, CN, SH, SR⁹, SO₃H, SO₃R⁹, SO₂R⁹, OSO₃R⁹, OR, CO₂R⁹, CONH₂, CONHR⁹, CONHR, CON(R⁹)₂, NHR, OPO₃H₃, CONR⁹R, NR⁹R or NO₂, wherein R⁸ is a moiety from TABLE 1 and each R⁹ is independently unsubstituted C₁-C₁₀ alkyl.

In other embodiments, the compound has one of the following Formulas Ia or Ib:

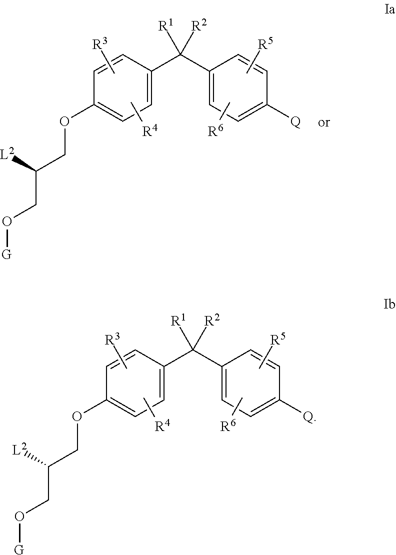

In certain embodiments of the foregoing, G¹ is a linear or branched, aromatic or non-aromatic cyclic, substituted or unsubstituted, saturated or unsaturated C₂-C₁₀ alkyl;

In some embodiments of the foregoing, M is H, F, Cl, Br, CH₂OH, CH₂F, CH₂Cl, CHCl₂, CCl₃, CH₂Br, CHBr₂ or CBr₃ and L is H or A-D, and in other embodiments M is H, OH, F, Cl, Br, CH₂OH, CH₂F, CCl₃, CH₂Br, CHBr₂ or CBr₃. In still other embodiments, M is H, OH, CH₂OH, CCl₃, CHBr₂ or CBr₃.

In other embodiments or the foregoing compound, G is methyl, ethyl or a C₄-C₂₀ alkyl. For example, in some embodiments the C₄-C₂₀ alkyl is saturated and in other embodiments the C₄-C₂₀ alkyl is unsaturated.

In yet other embodiments of the foregoing, L is A-D or —CH₂-A-D, for example in some embodiments L is A-D. In certain specific embodiments, L is A-D or —CH₂-A-D and G is methyl, ethyl or a C₄-C₂₀ alkyl. In some embodiments, L is H.

In still other embodiment, G is a linear, branched or non-aromatic cyclic, substituted or unsubstituted, unsaturated C₁-C₂₀ alkyl, wherein one or more carbon atoms of the unsaturated C₁-C₂₀ alkyl may optionally be replaced with an oxygen atom.

In yet other embodiments, G is a linear, branched or non-aromatic cyclic, substituted or unsubstituted, saturated C₁-C₂₀ alkyl, wherein one or more carbon atoms of the C₁-C₂₀ alkyl may optionally be replaced with an oxygen atom, for example in certain embodiments none of the carbon atoms of the C₁-C₂₀ alkyl are replaced with an oxygen atom.

In certain embodiments, the compound has one of the following Formulas II or III:

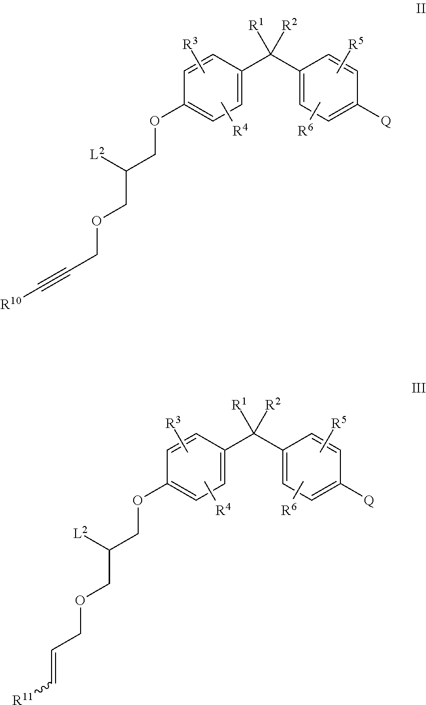

wherein R¹⁰ and R¹¹ are each independently H or linear or branched, substituted or unsubstituted, saturated or unsaturated C₁-C₆ alkyl.

For example, in certain embodiments the compound has one of the following Formulas IIa, IIb, IIIa or IIIb:

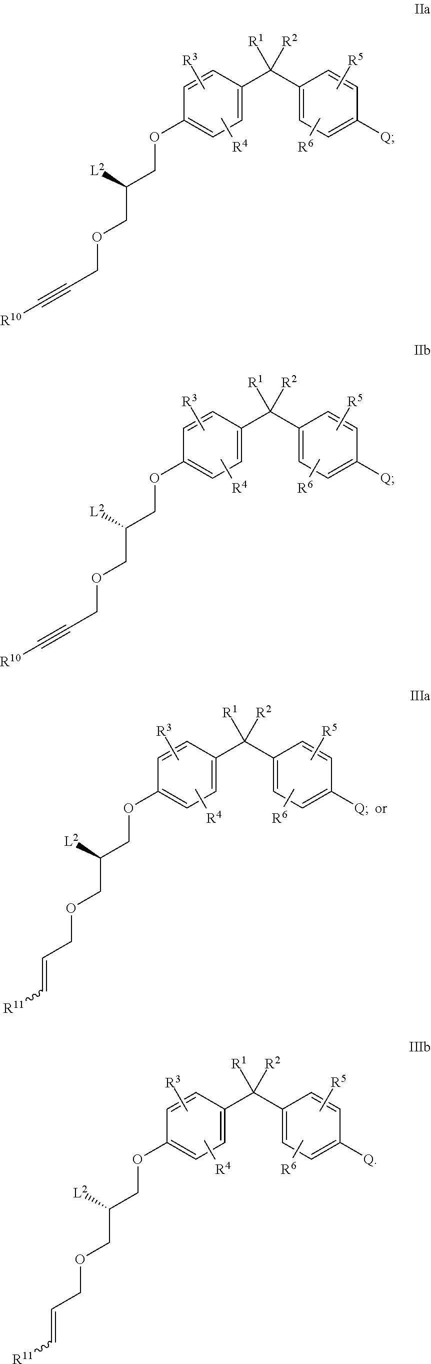

In other examples, the compound has one of the following Formulas IV or V:

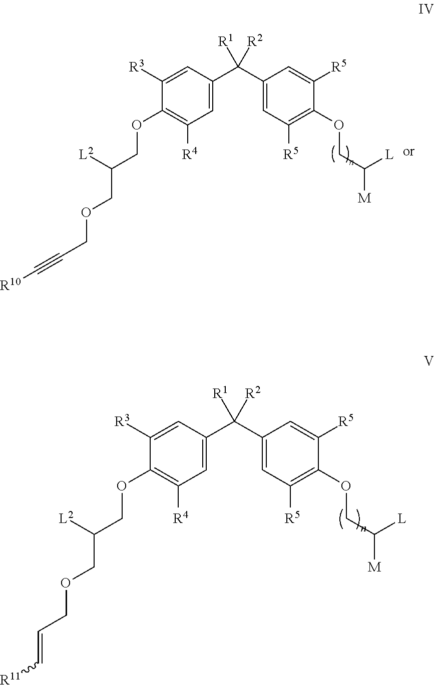

For example, in certain embodiments the compound has one of the following structures IVa, IVb, IVc, IVd, Va, Vb, Vc or Vd:

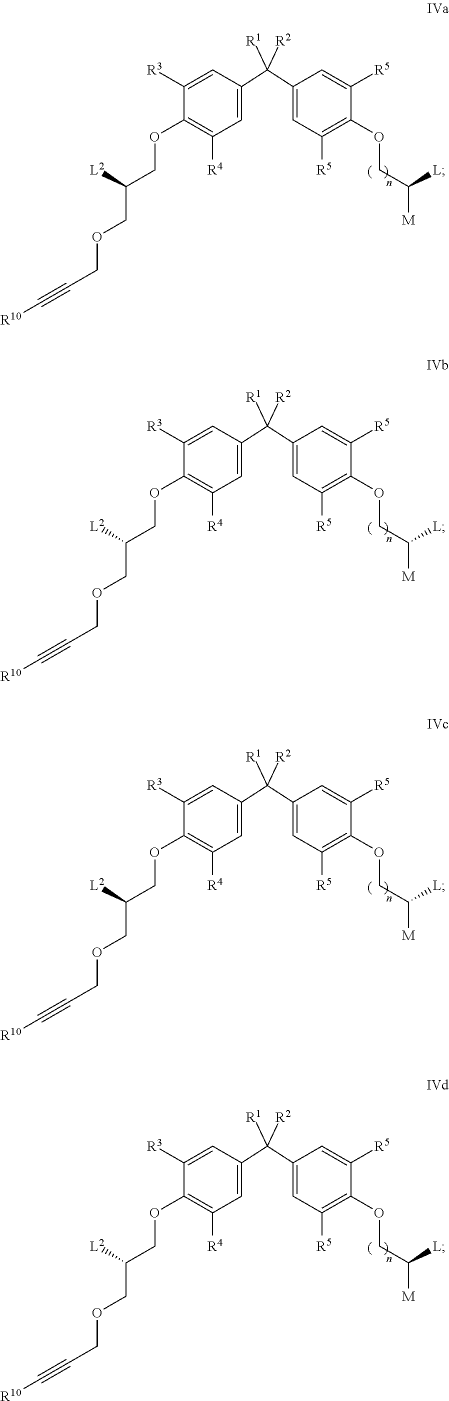

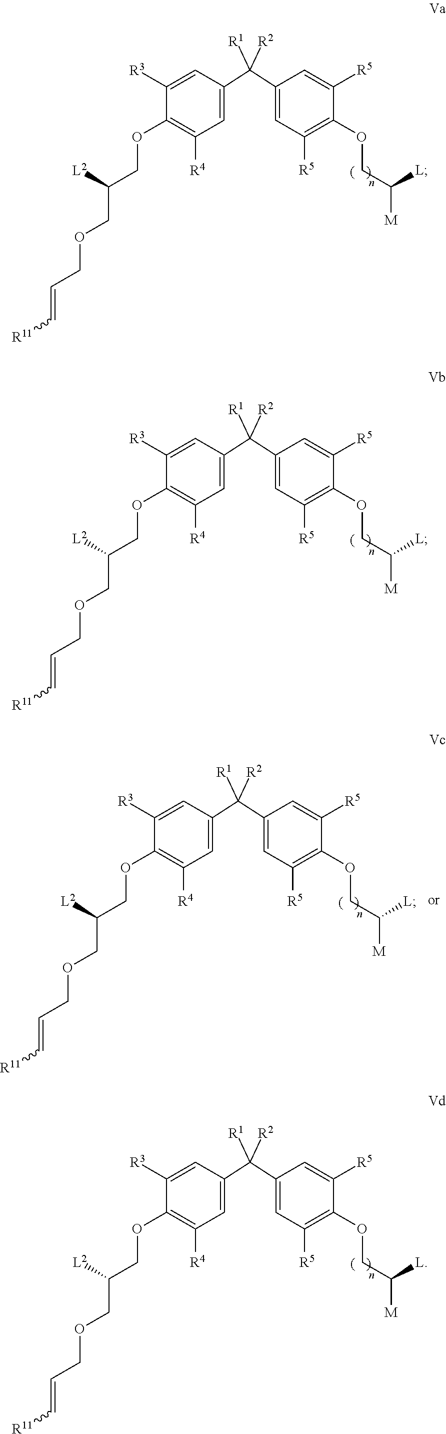

In other embodiments, the compound has one of the following Formulas VI or VII:

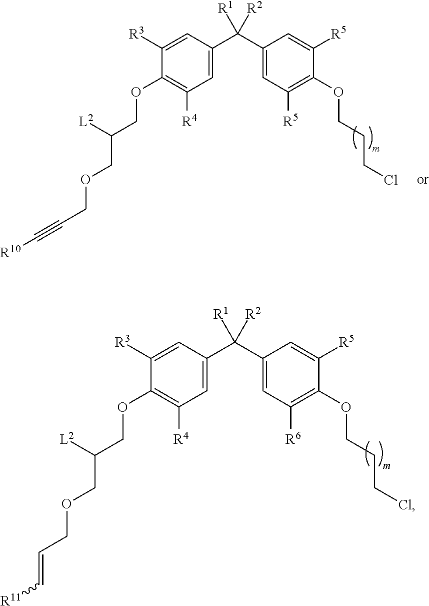

wherein m is 0, 1, 2, 3, 4 or 5.

For example, in certain specific embodiments the compound has one of the following Formulas VIIa, VIIb, VIIa or VIIb:

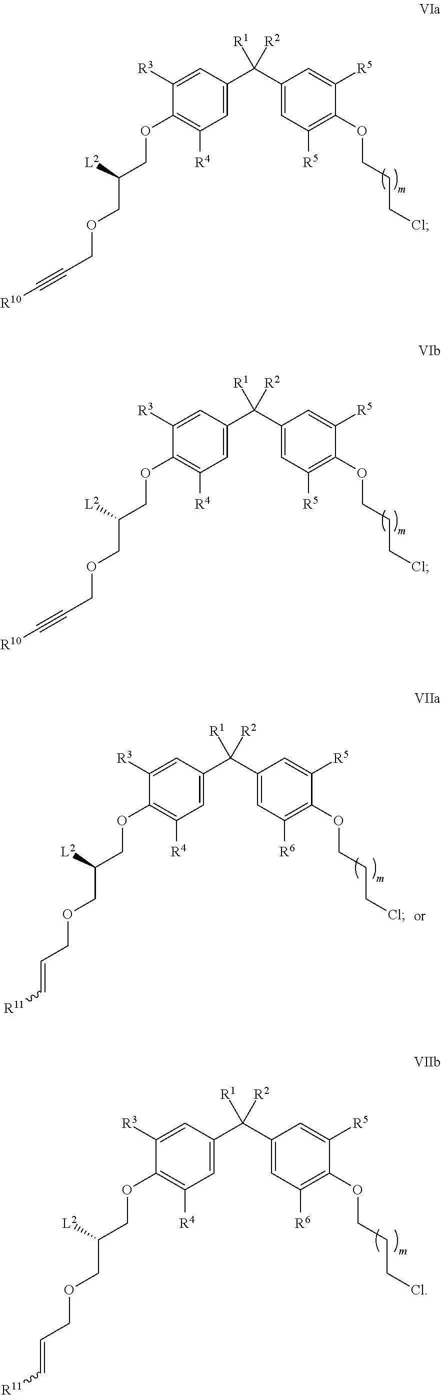

In certain embodiments, the compound has one of the following Formulas VIII, IX or XI:

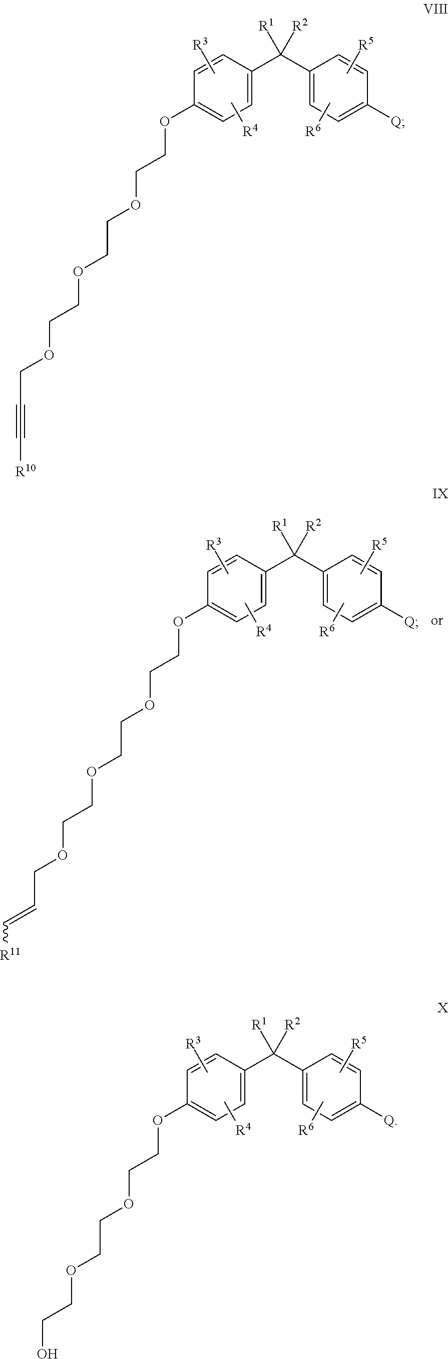

In other embodiments, the compound has one of the following Formulas XI, XII, or XIII:

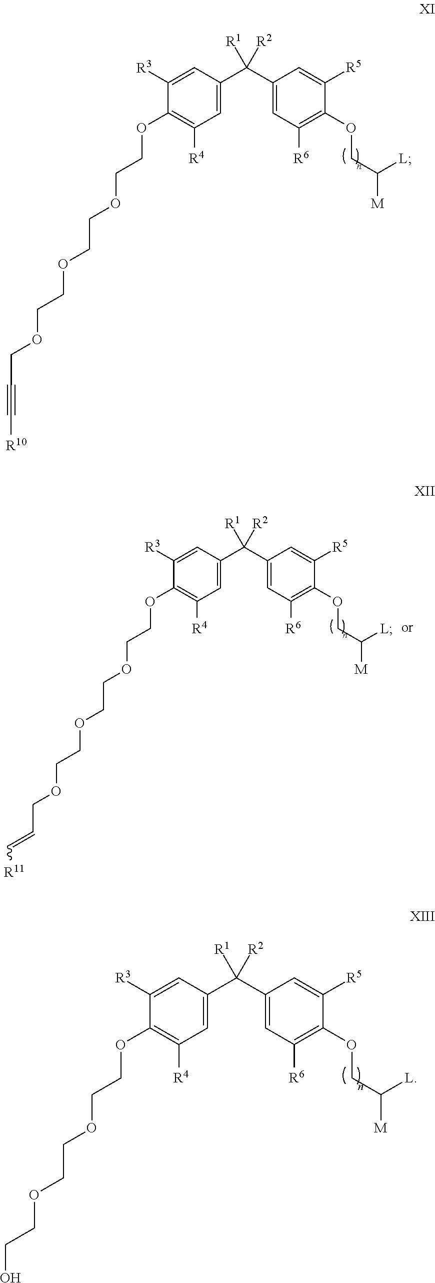

For example, in other aspects the compound has one of the following Formulas XIa, XIb, XIIa, XIIb, XIIIa or XIIIb:

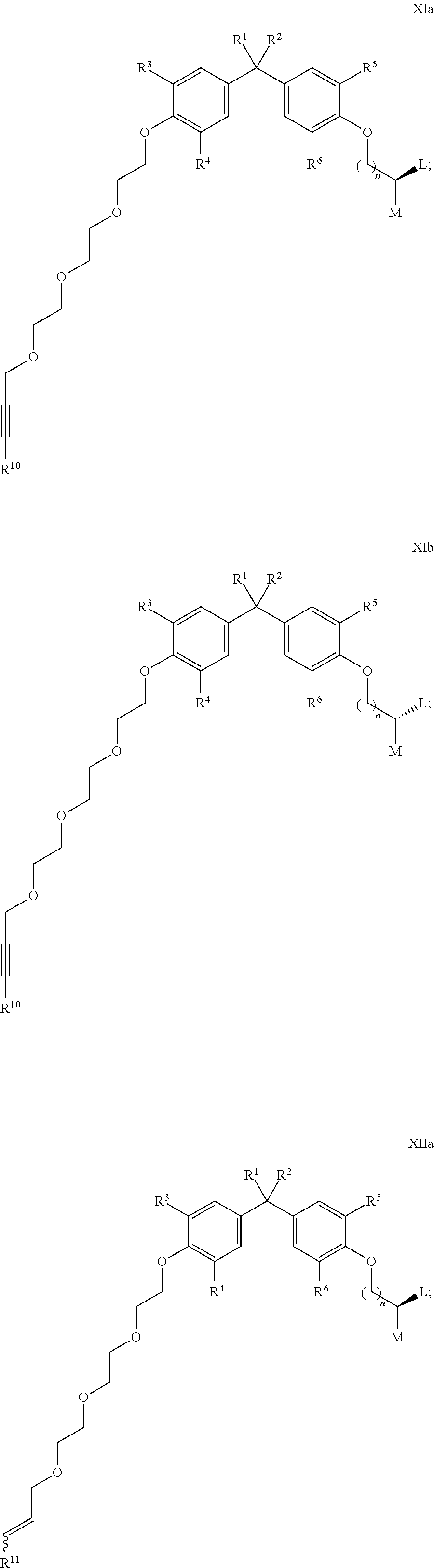

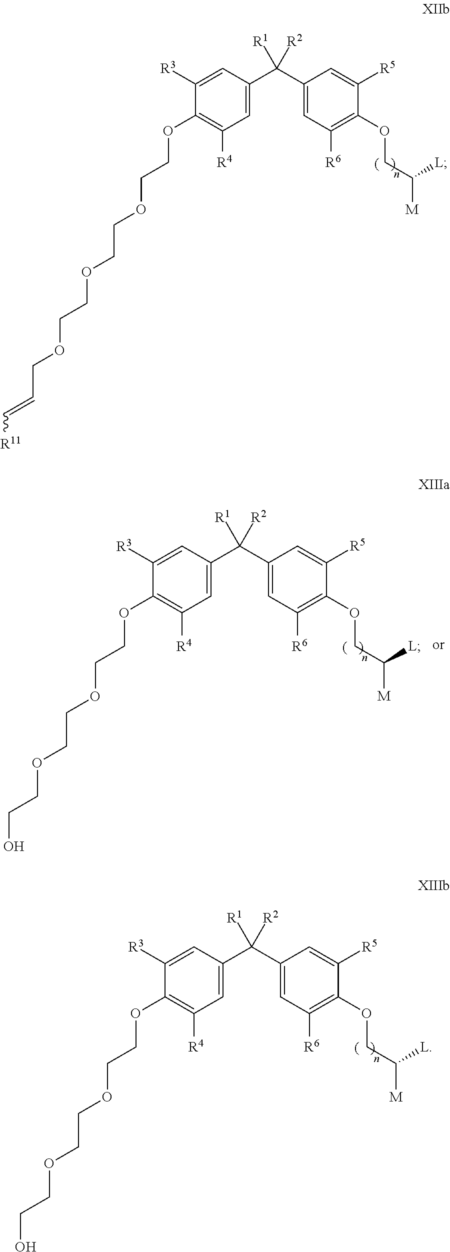

In other aspects, the compound has one of the following Formulas XIV, XV or XVI:

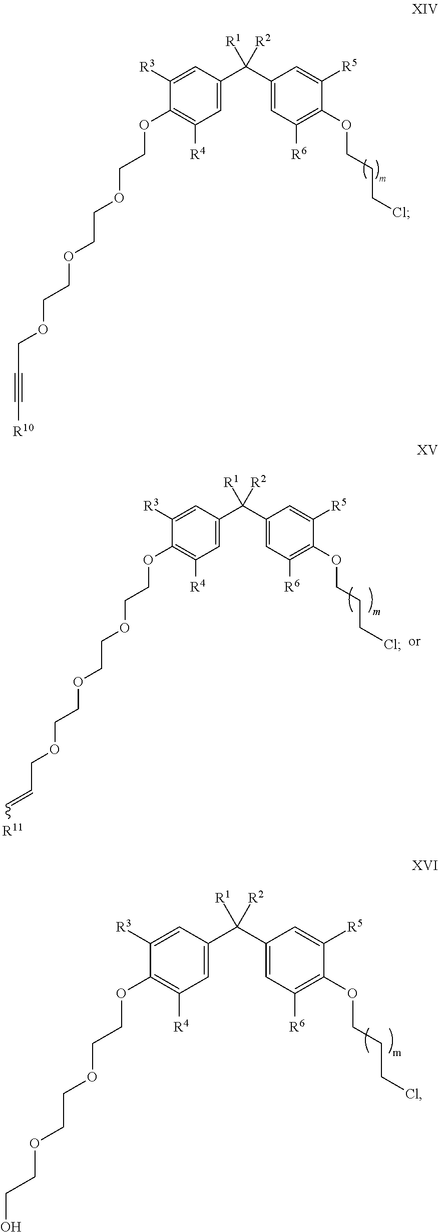

wherein m is 0, 1, 2, 3, 4 or 5.

In certain embodiments, G is a linear, branched or non-aromatic cyclic, substituted or unsubstituted, saturated C₁-C₁₀ alkyl. For example, in certain embodiments the compound has one of the following Formulas XVII, XVIII or XIX:

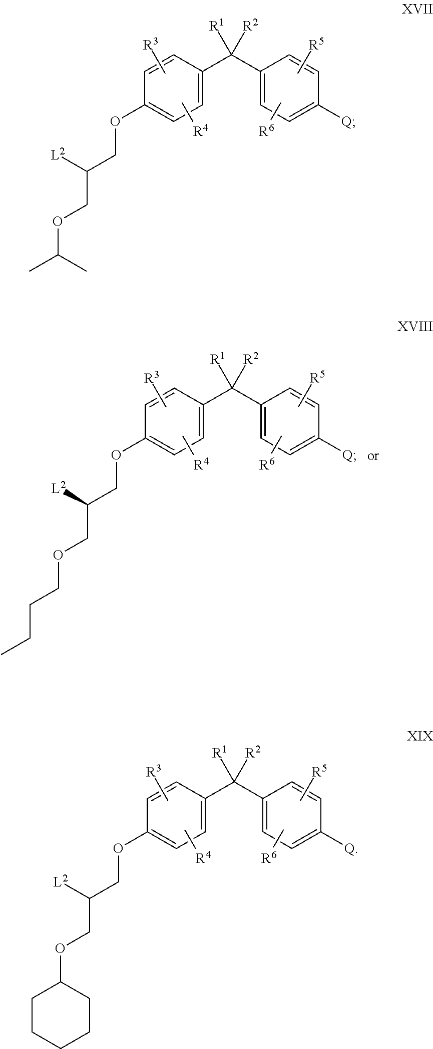

In other further embodiments, the compound has one of the following Formulas XVIIa, XVIIb, XVIIIa, XVIIIb, XIXa or XIXb:

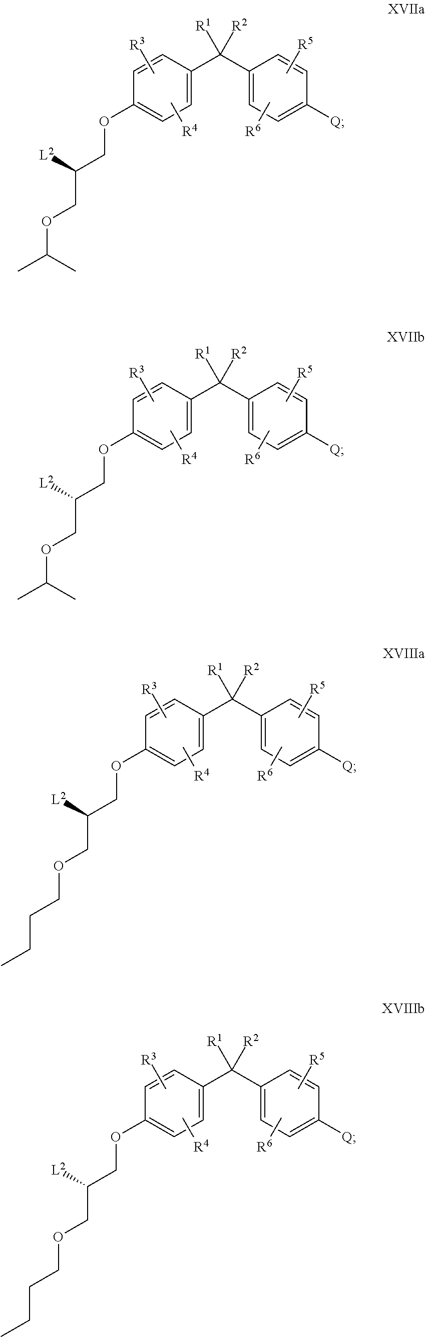

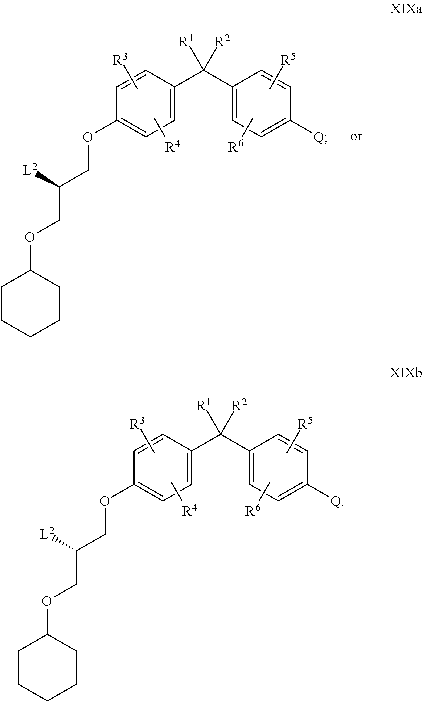

In other aspects, the compound has one of the following Formulas XX, XXI or XXII:

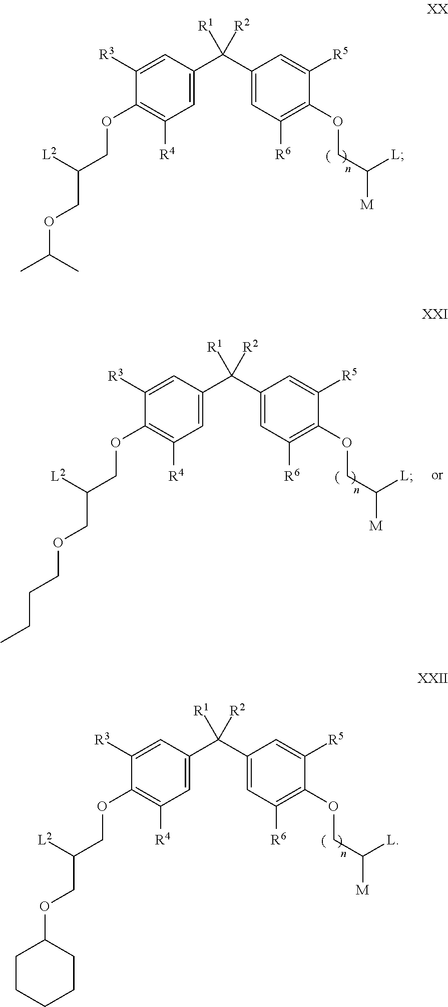

For example, in certain specific embodiments the compound has one of the following Formulas XXa, XXb, XXc, XXd, XXIa, XXIb, XXIc, XXId, XXIIa, XXIIb, XXIIc or XXIId:

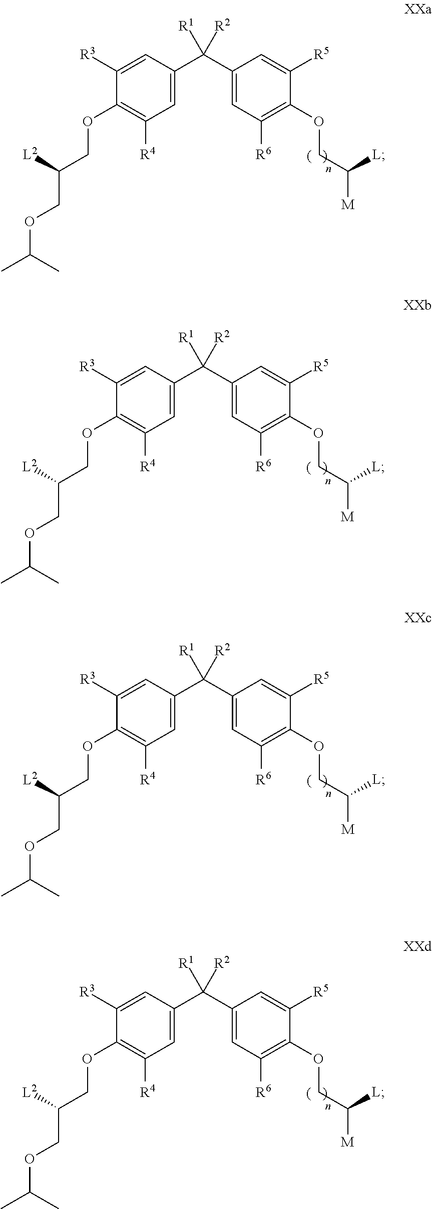

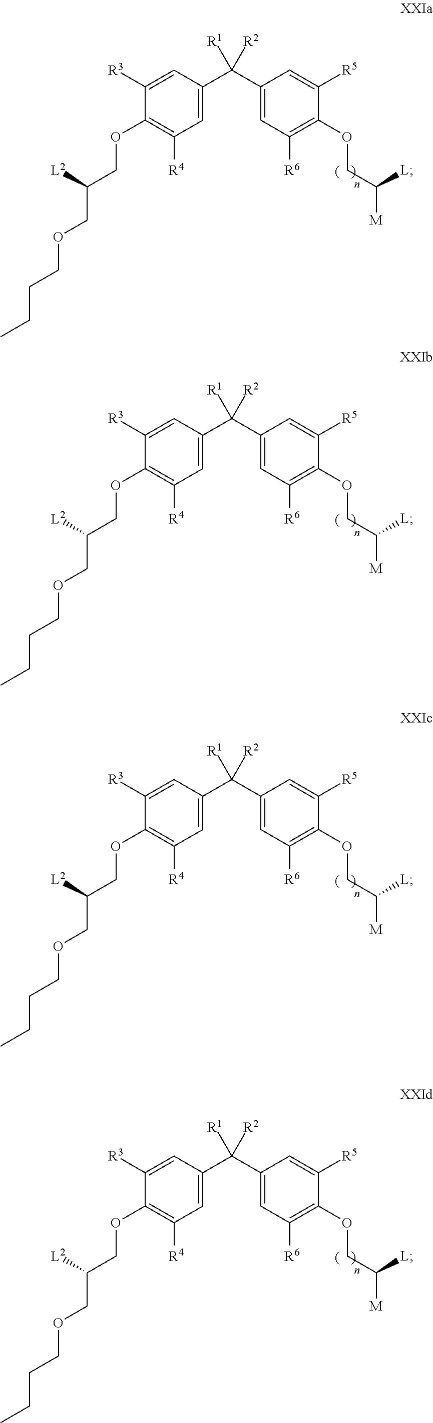

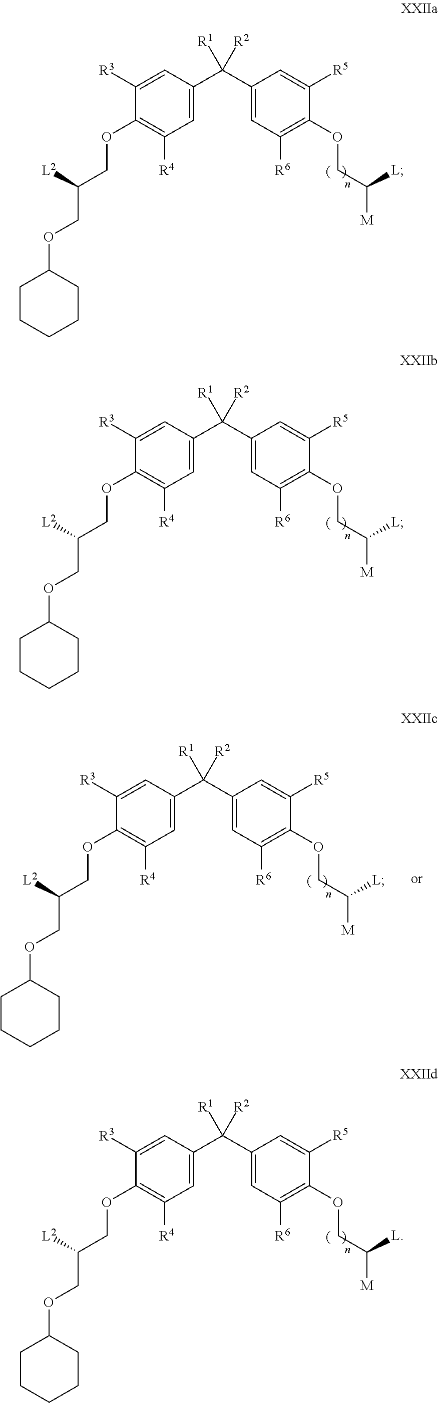

In yet other embodiments, the compound has one of the following Formulas XXIII, XXIV or XXV:

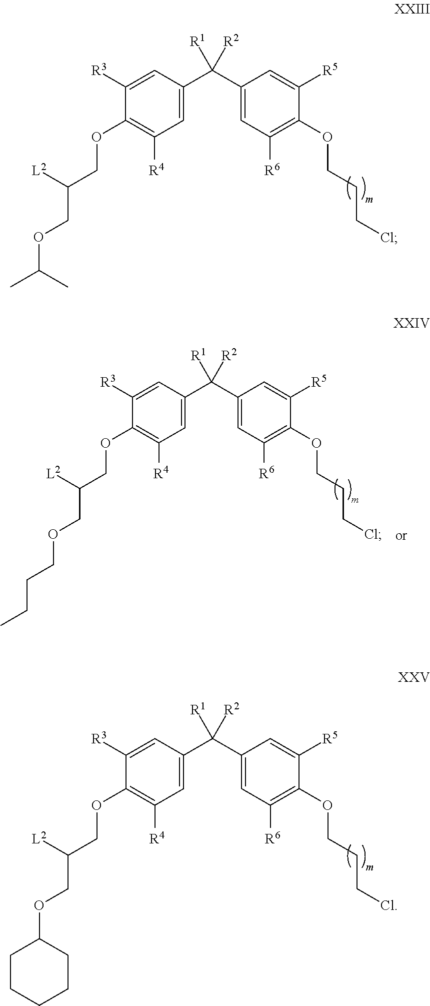

For example, in certain embodiments the compound has one of the following Formulas XXIIIa, XXIIIb, XXIVa, XXIVb, XXVa or XXVb:

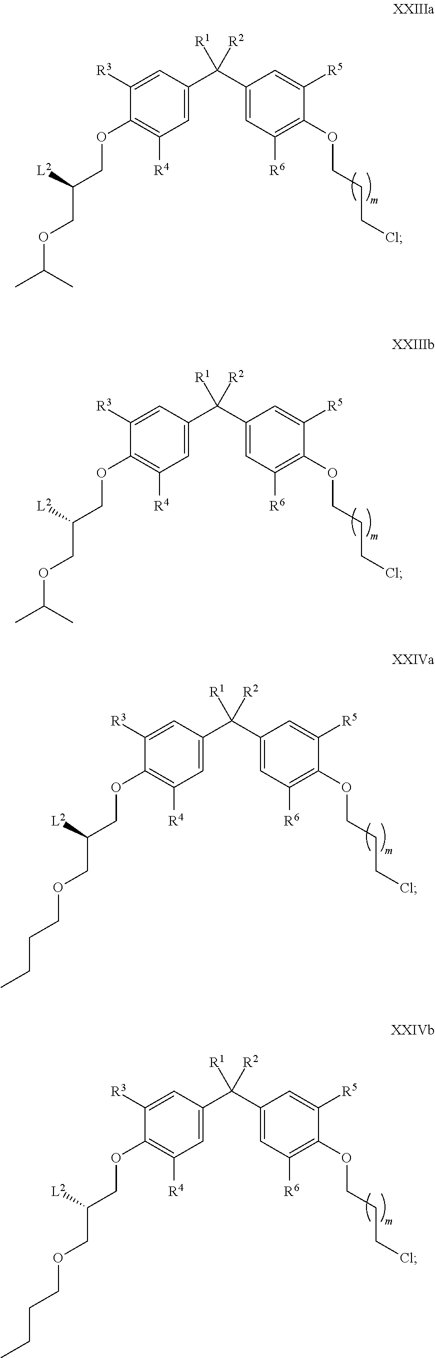

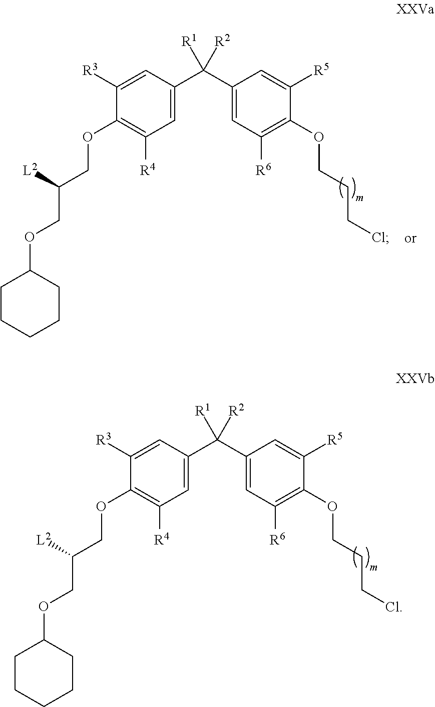

In some embodiments, L² is OH. In other embodiments, R¹⁰ or R¹¹ is H. In other examples, one of R¹ or R² is methyl. For example, in certain specific embodiments R¹ and R² are each methyl.

In other aspects, at least one of R³, R⁴, R⁵ or R⁶ is hydrogen. For example, in certain specific embodiments R³, R⁴, R⁵ and R⁶ are each hydrogen. In other examples, R¹ and R² are each methyl and R³, R⁴, R⁵ and R⁶ are each hydrogen.

In yet other embodiments, at least one of R³, R⁴, R⁵ or R⁶ is methyl. In other aspects, R³, R⁴, R⁵ and R⁶ are each methyl. In other examples, R¹ and R² are each methyl and R³, R⁴, R⁵ and R⁶ are each methyl.

In some embodiments, at least one of R³, R⁴, R⁵ or R⁶ is bromo. For example, in certain embodiments R³, R⁴, R⁵ and R⁶ are each bromo. In other embodiments, R¹ and R² are each methyl and R³, R⁴, R⁵ and R⁶ are each bromo.

In some embodiments, at least one of R³, R⁴, R⁵ or R⁶ is chloro. For example, in certain embodiments R³, R⁴, R⁵ and R⁶ are each chloro. In other examples, R¹ and R² are each methyl and R³, R⁴, R⁵ and R⁶ are each chloro.

In other aspects, at least one of R³, R⁴, R⁵ or R⁶ is fluoro. For example, in certain embodiments R³, R⁴, R⁵ and R⁶ are each fluoro. In other examples, R¹ and R² are each methyl and R³, R⁴, R⁵ and R⁶ are each fluoro.

In some embodiments, L is OH. In other examples, J is G′, O, CH₂, CHG¹, CG¹₂, NH, SO, or NR. For example, in certain specific embodiments J is O.

In other embodiments, M is F, Cl, Br, CH₂OH, CH₂Cl, CHCl₂, CCl₃, CH₂Br, CHBr₂ or CBr₃. For example, in certain specific embodiments M is CH₂F, CH₂Cl or CH₂Br. In other aspects, M is CH₂Cl. In other examples, M is CH₂F. In other further embodiments, L is OH and M is CH₂Cl. In some embodiments, M is CH₂Omethyl, CH₂Oisopropyl, CH₂Obutyl or CH₂Ocyclohexyl. In certain embodiments, M is H.

In some embodiments, L is H. In other embodiments, L is A-D. For example, in certain embodiments A is O. In other aspects, D is H, R, or a moiety from TABLE 1. In other examples, D is H. In other aspects, D is R, and In yet other embodiments, D is a moiety from TABLE 1, for example,

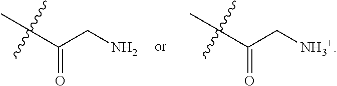

In certain embodiments, n is 0. In other embodiments, n is 1, 2, 3, 4, or 5. In yet other embodiments, n is 1.

In some embodiments, L² is OH. In other examples, L² is A²-D². In other aspects, A² is O. In yet other embodiments, D² is H, R or a moiety from TABLE 1. For example, in certain specific embodiments D² is H. In other specific embodiments, D² is R. In yet other specific embodiments, D² is a moiety from TABLE 1, for example,

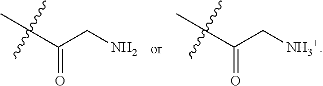

In some embodiments, m is 0. In yet other embodiments, m is 1, 2, 3, 4, or 5. In still other embodiments, m is 1.

In certain embodiments L² is OH and R¹⁰ and R¹¹ are each hydrogen. In yet other embodiments, L² is OH, R¹⁰ and R¹¹ are each hydrogen, R¹ and R² are each methyl and R³, R⁴, R⁵ and R⁶ are each hydrogen. In other further embodiments, L² is OH, R¹⁰ and R¹¹ are each hydrogen, R¹ and R² are each methyl and R³, R⁴, R⁵ and R⁶ are each methyl. In yet other further embodiments, L² is OH, R¹⁰ and R¹¹ are each hydrogen, R¹ and R² are each methyl and R³, R⁴, R⁵ and R⁶ are each bromo. In yet other embodiments, L² is OH, R¹⁰ and R¹¹ are each hydrogen, R¹ and R² are each methyl and R³, R⁴, R⁵ and R⁶ are each chloro. In still other further embodiments, L² is OH, R¹⁰ and R¹¹ are each hydrogen, R¹ and R² are each methyl and R³, R⁴, R⁵ and R⁶ are each fluoro. In other aspects, G is a linear, branched or non-aromatic, substituted or unsubstituted, saturated alkyl and L² is OH.

In certain embodiments, Q is

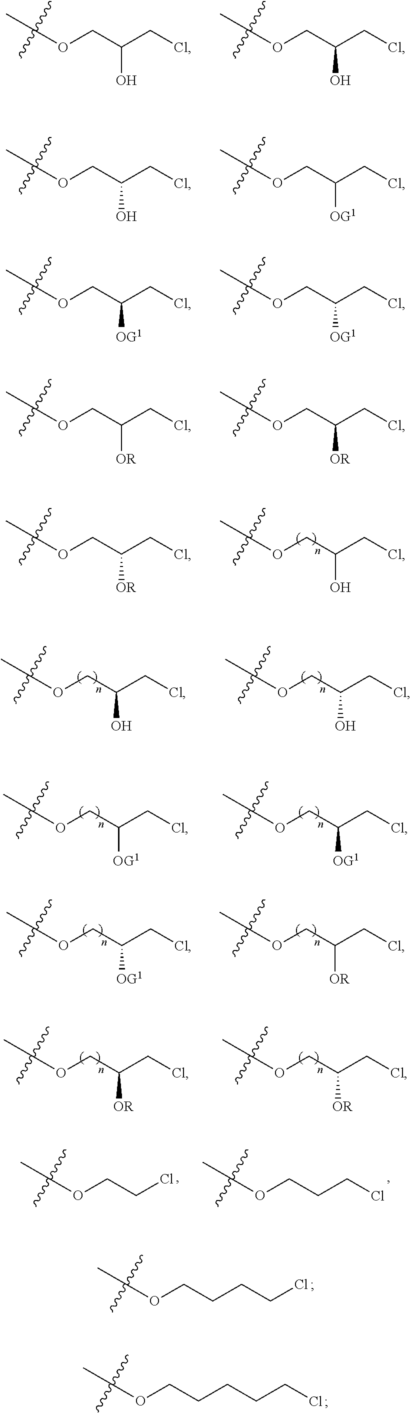

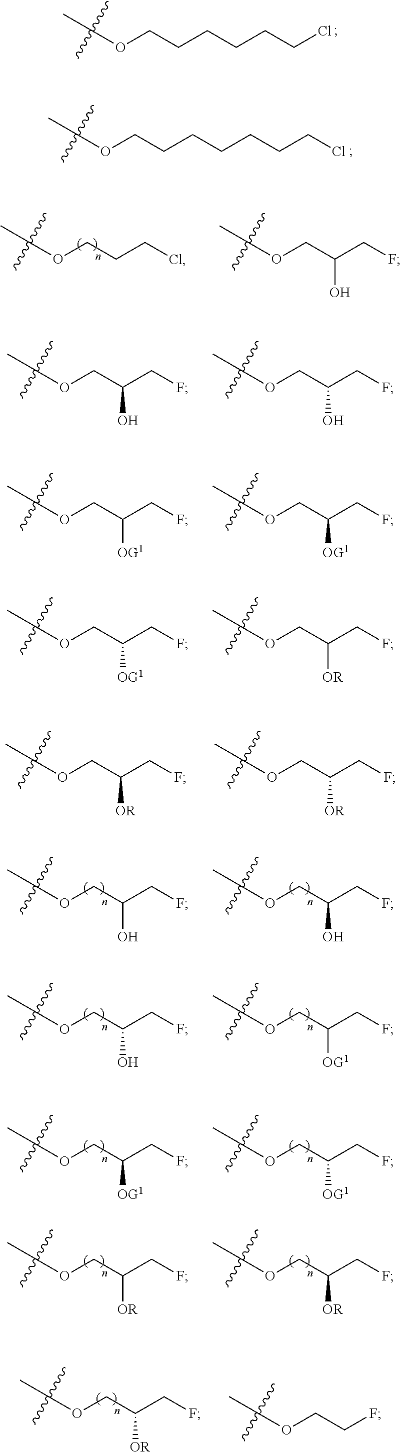

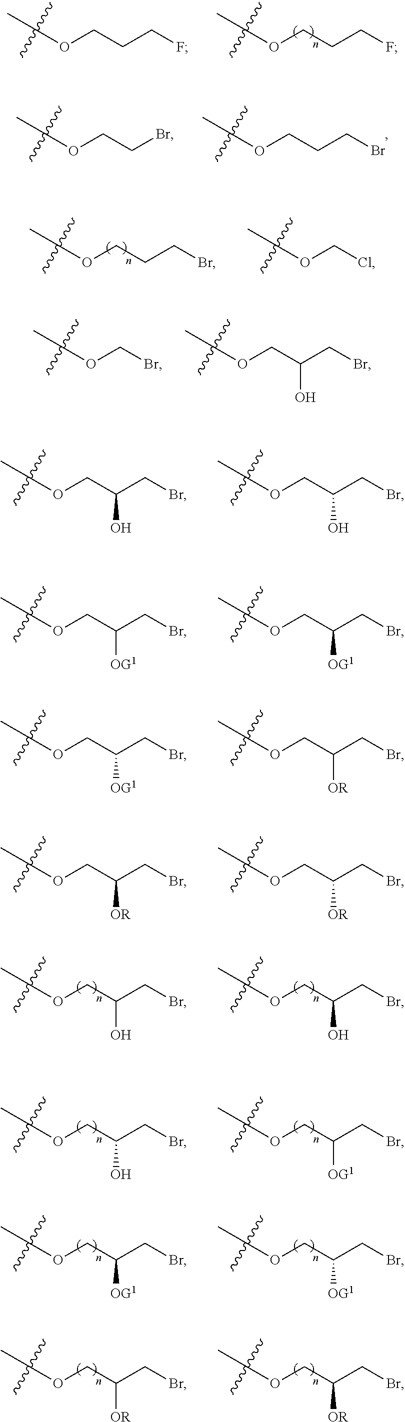

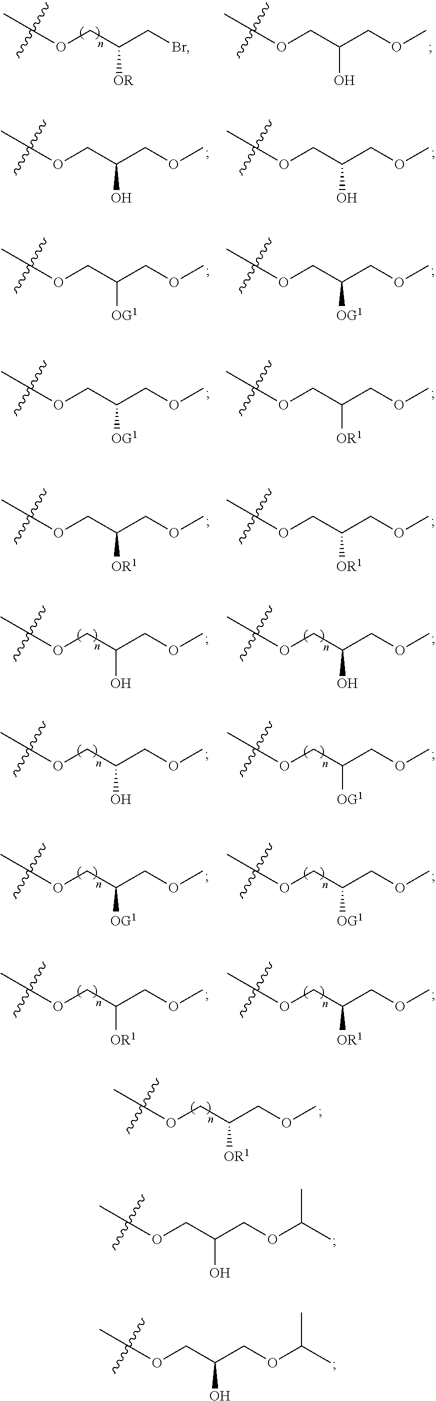

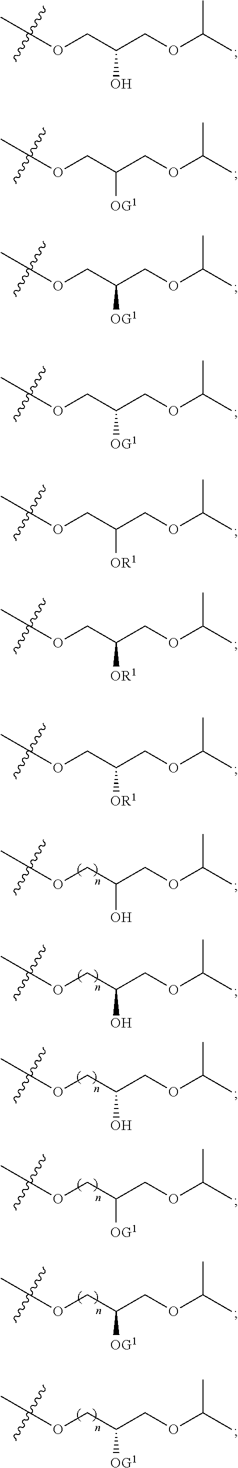

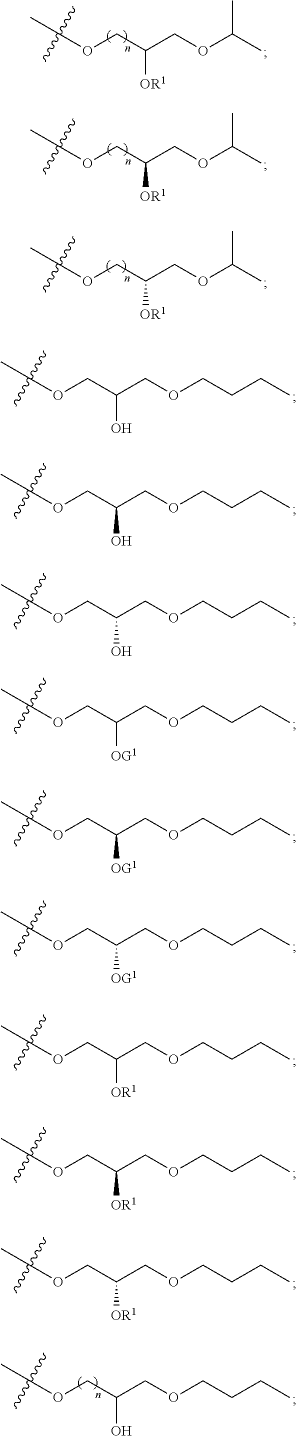

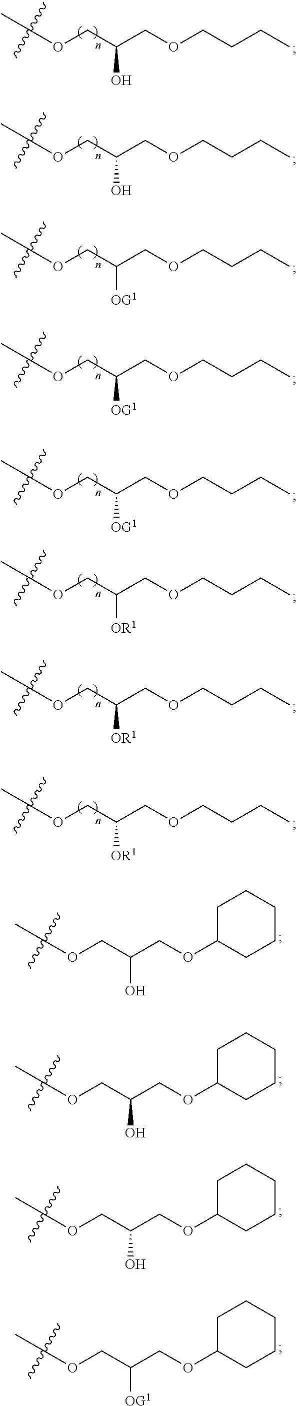

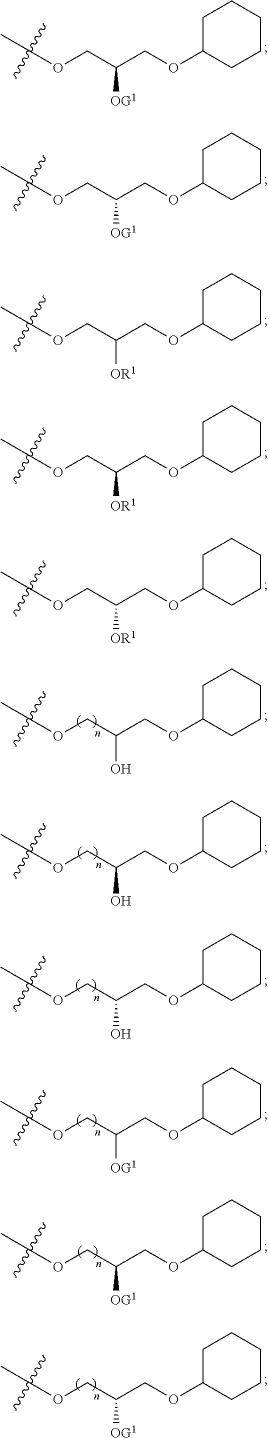

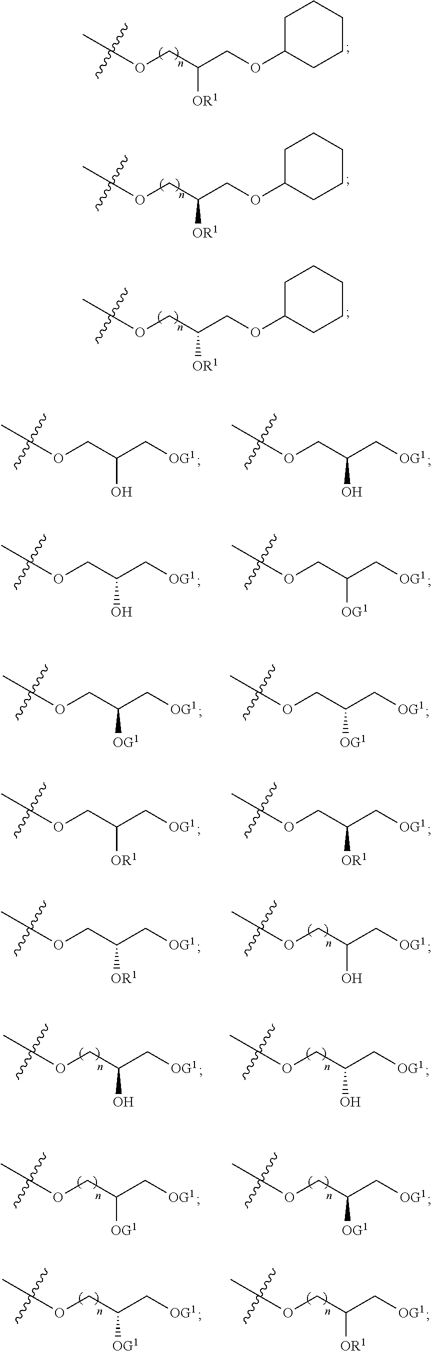

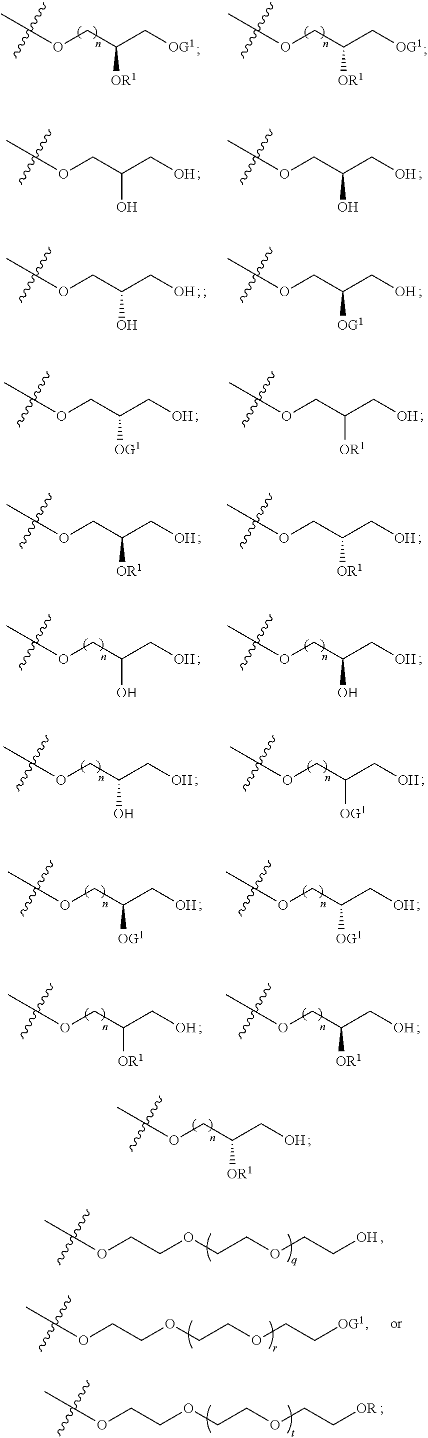

n is 0, 1, 2, 3, 4, 5, 6, 7 or 8;

each of q, r, and t is independently 0, 1, 2, 3, 4, 5, 6 or 7;

each G¹ is independently linear or branched, substituted or unsubstituted, saturated or unsaturated C₁-C₁₀ alkyl, wherein the optional substituent is oxo, OR⁸, COOH, OH, F, Cl, Br, I, NH₂, CN, SH, SO₃H, CONH₂, OPO₃H₃ or NO₂.

In other examples, Q is

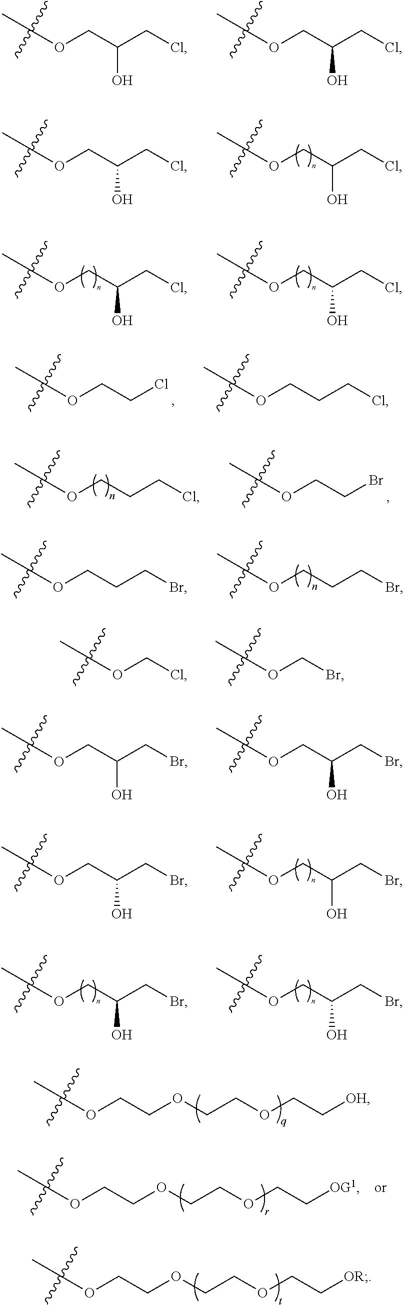

n is 0, 1, 2, 3,4, 5, 6, 7 or 8;

each of q, r, and t is independently 0, 1, 2, 3, 4, 5, 6 or 7; and

each G¹ is independently linear or branched, substituted or unsubstituted, saturated or unsaturated C₁-C₁₀ alkyl, wherein the optional substituent is selected from the group consisting of oxo, OR⁸, COOH, OH, F, Cl, Br, I, NH₂, CN, SH, SO₃H, CONH₂, OPO₃H₃, and NO₂.

In yet other embodiments, Q is

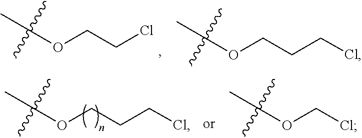

and

n is 0, 1, 2, 3, 4, 5, 6, 7 or 8.

In other yet embodiments, Q is

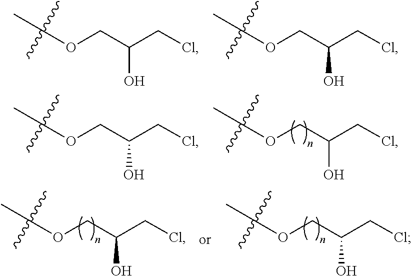

and

n is 0, 1, 2, 3, 4, 5, 6, 7 or 8.

In yet other embodiments, Q is

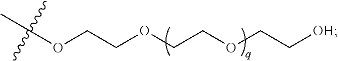

and

q is 0, 1, 2, 3, 4, 5, 6 or 7.

In other further embodiments, Q is

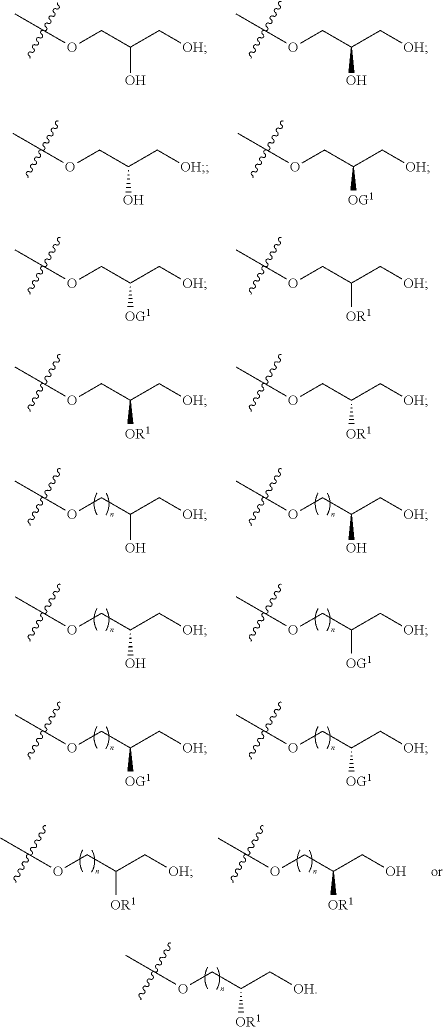

In other further embodiments, Q is

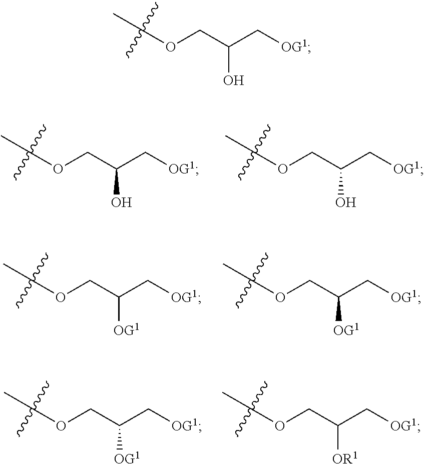

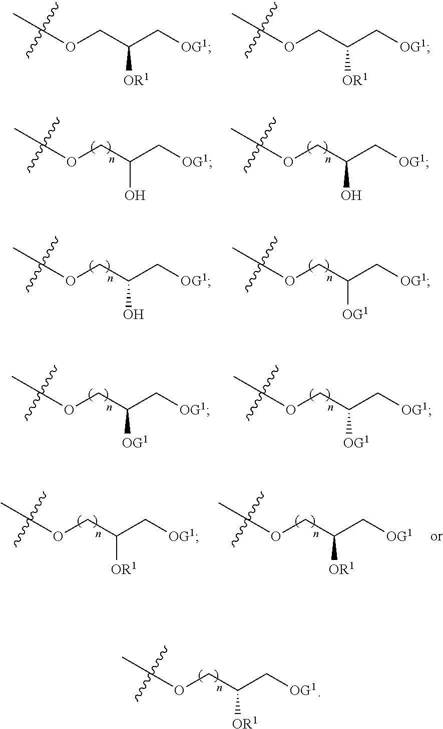

In other further embodiments, Q is

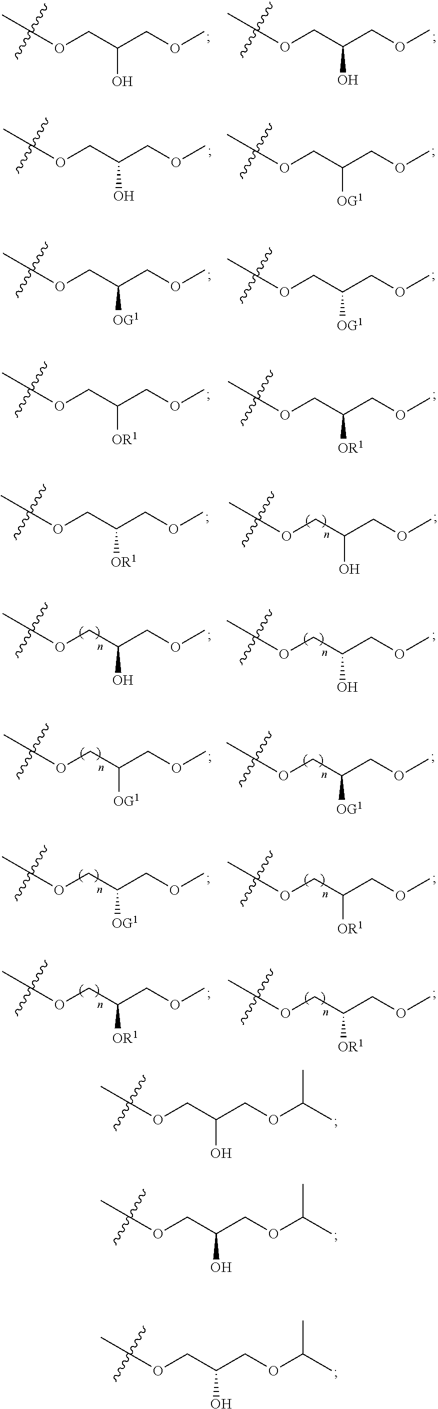

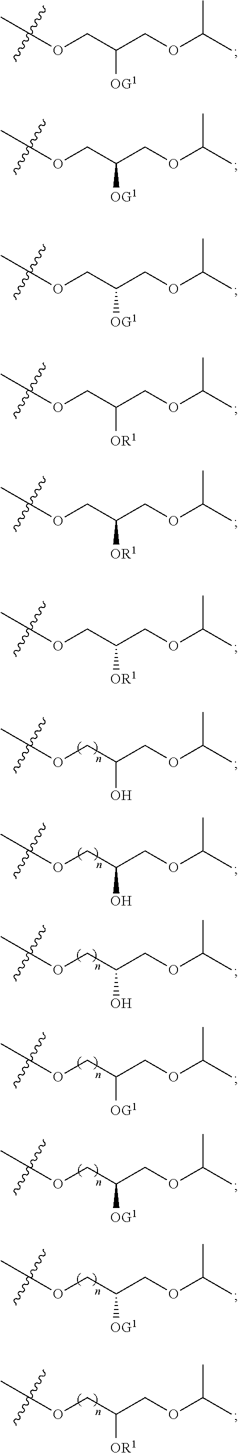

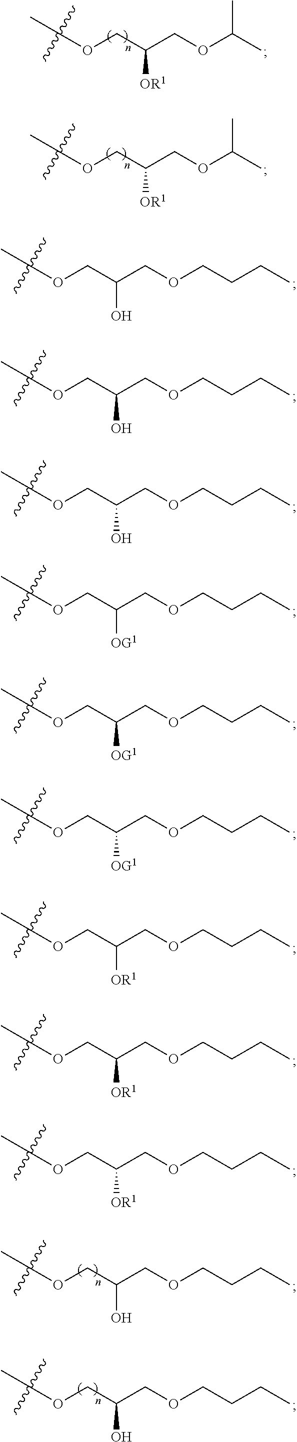

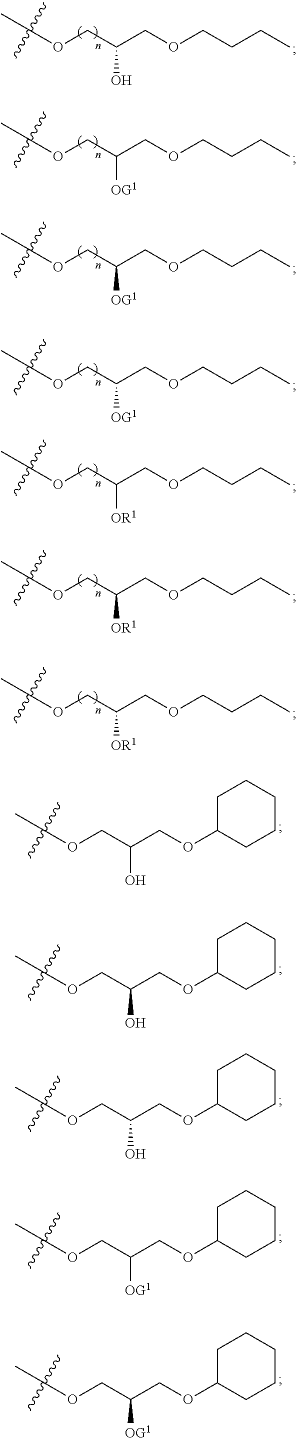

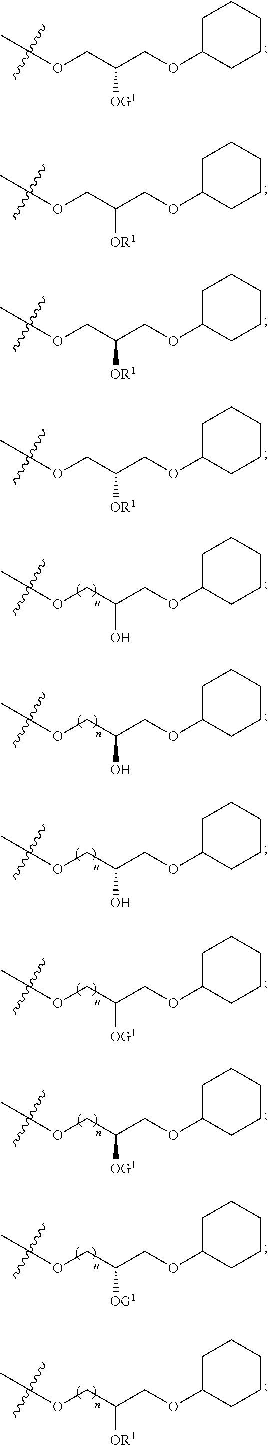

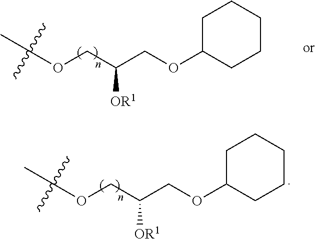

In certain embodiments, G, is CH₂CCH or CH═CH₂. In other certain embodiments, one or more of the OH groups of the compound is optionally substituted to replace the H with a moiety from TABLE 1, for example,

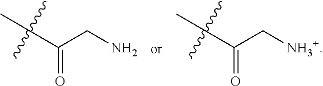

In other embodiments, the present disclosure provides a compound having one of the following structures:

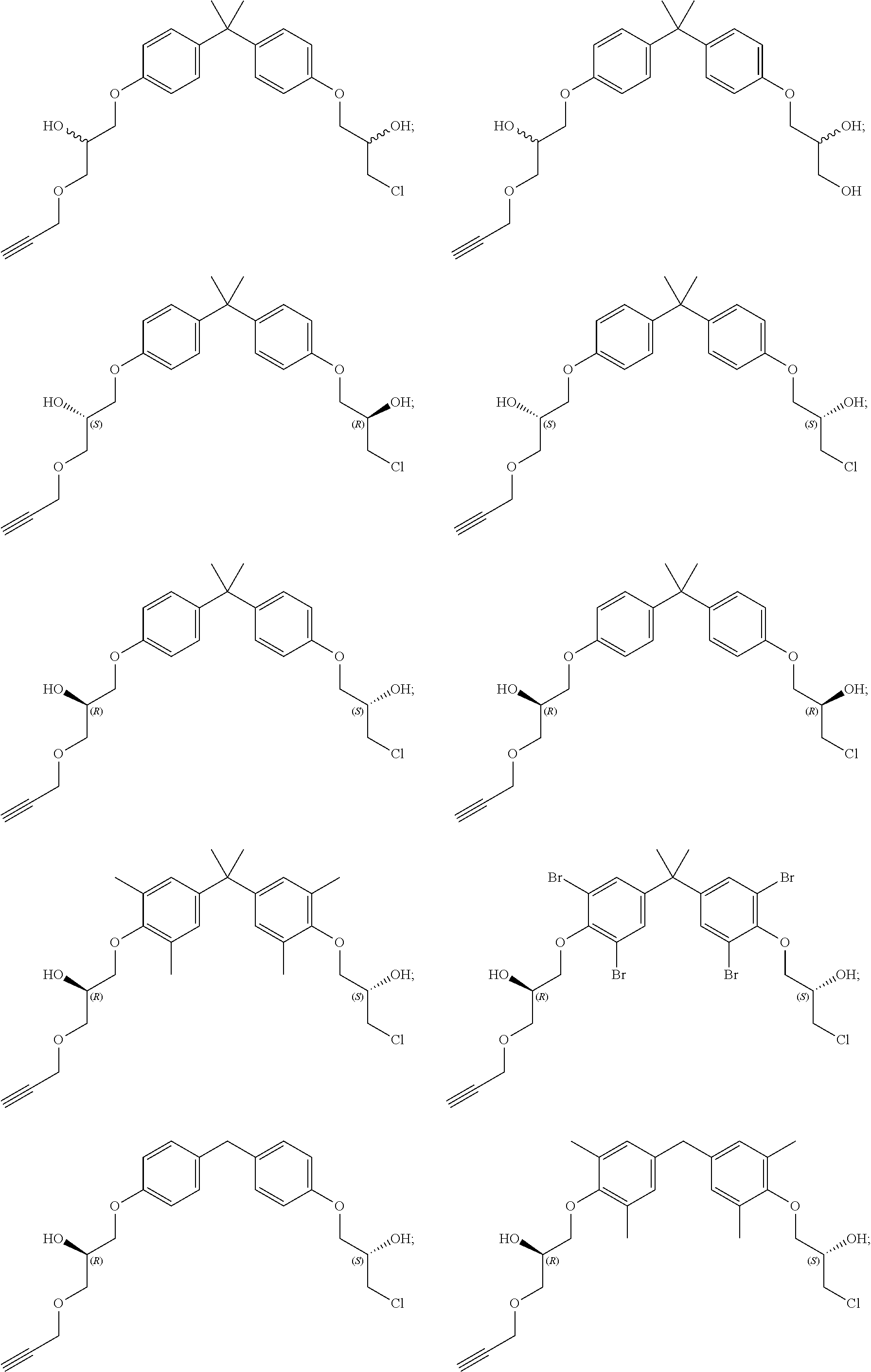

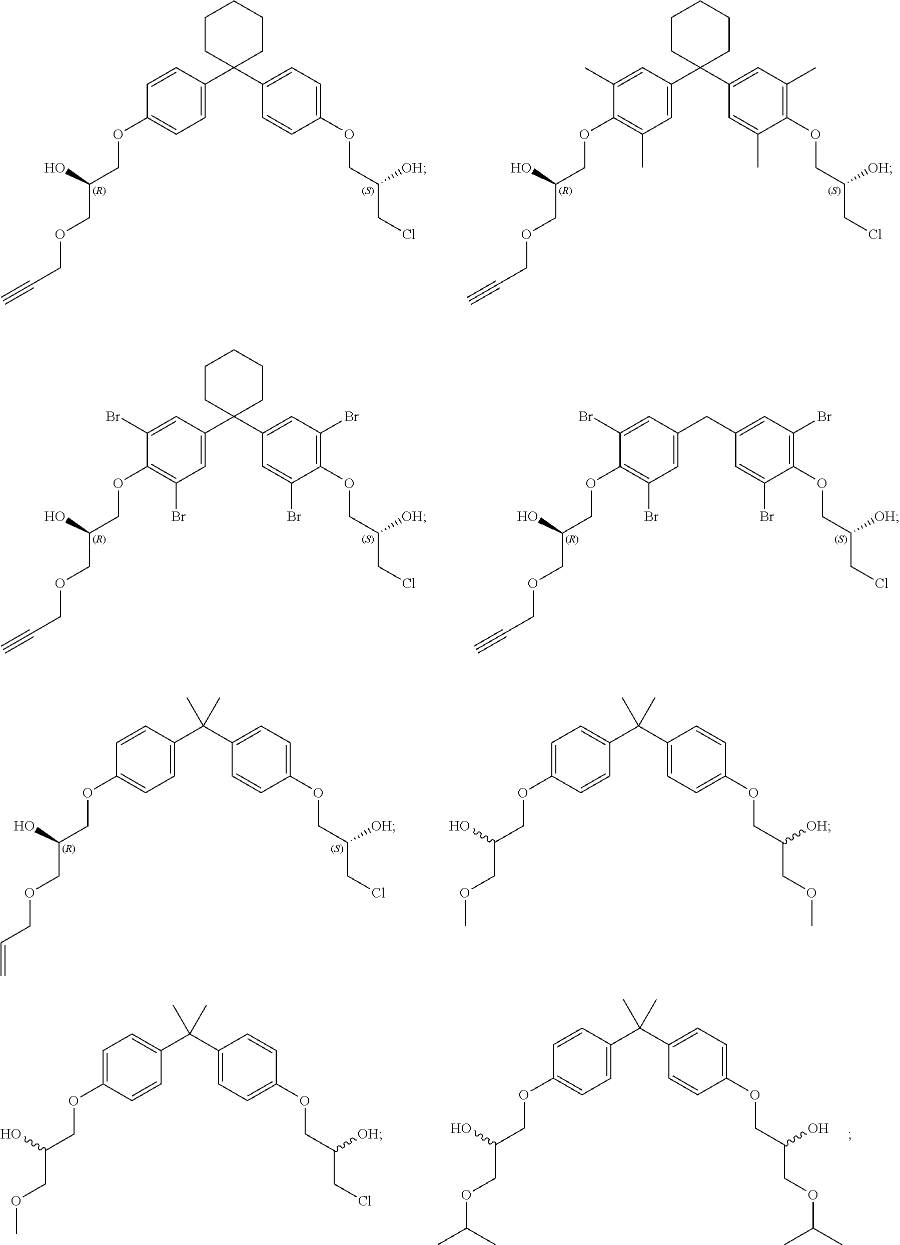

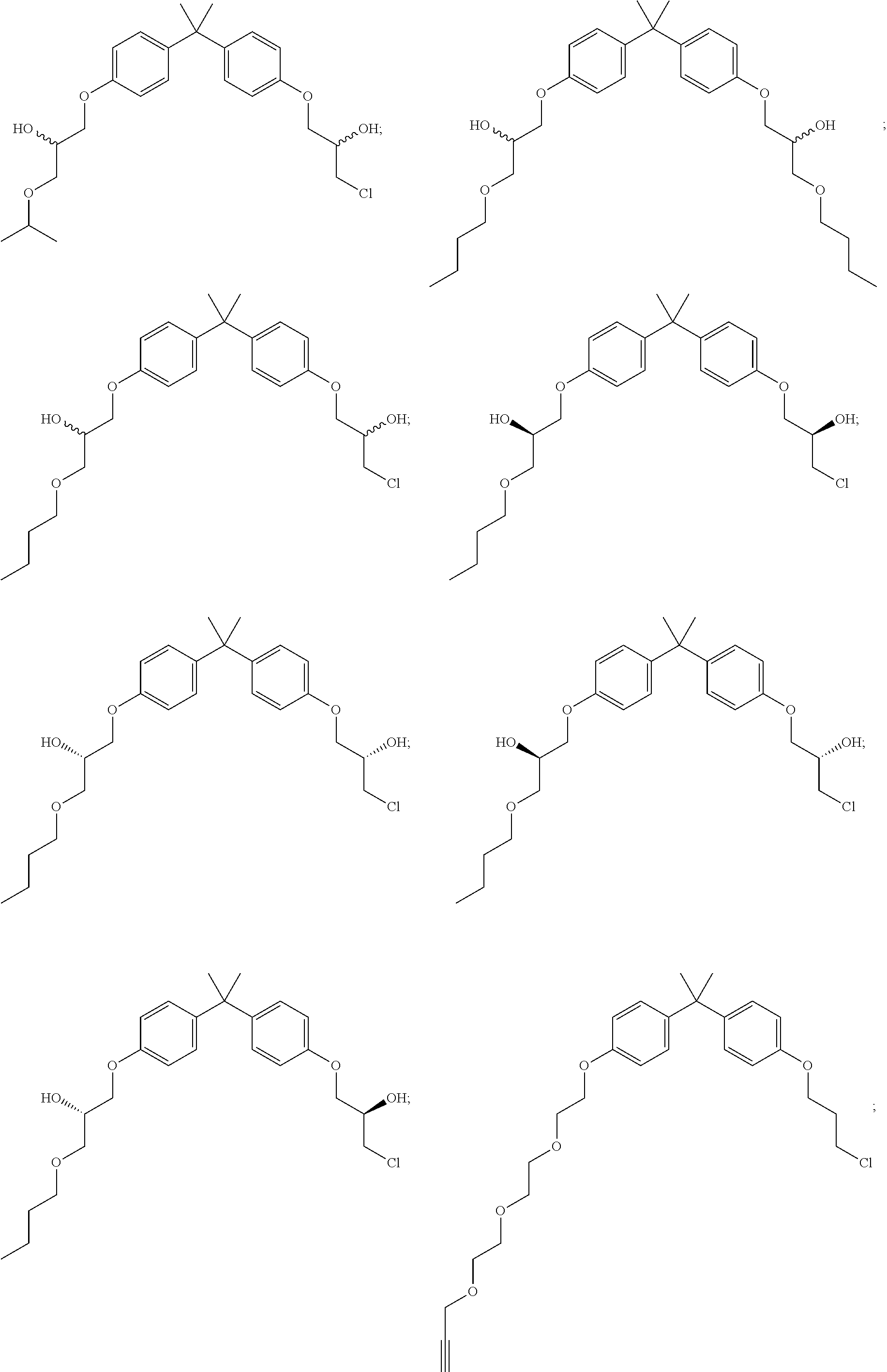

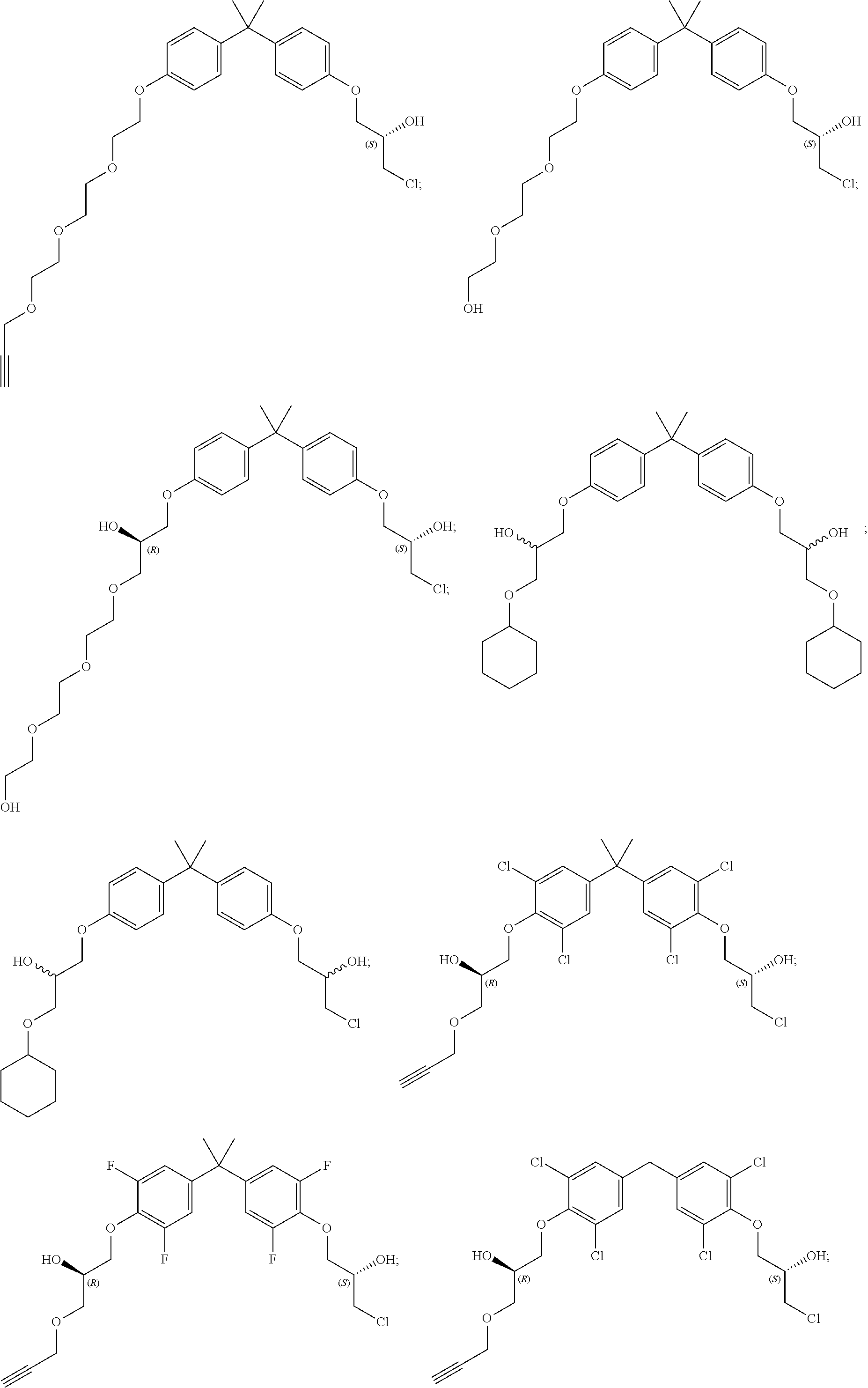

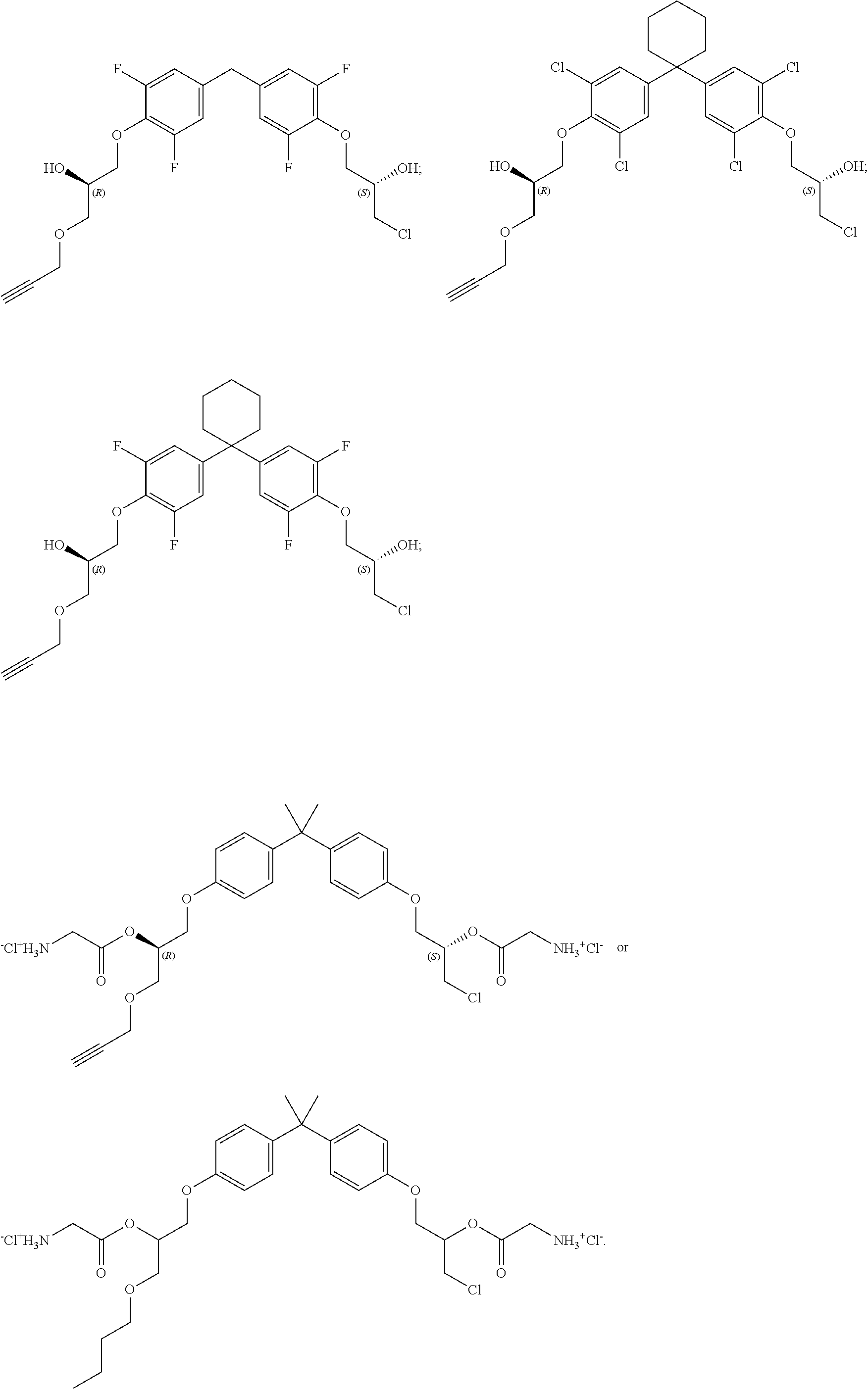

In certain embodiments, each J may independently be G¹, O, CH₂, CHG¹, CG¹₂, S, NH, NG¹, SO, SO₂, or NR. Each J may independently be G¹, O, CH₂, CHG¹, CG¹₂, S, NH, or NG¹. Each J may independently be O, S, NH, NG¹, SO, SO₂, or NR. Each J may independently be O, S, SO, or SO₂. Each J may independently be O, NH, NG¹, or NR. Each J may independently be S, NH, NG¹, SO, SO₂, or NR. Each J may independently be S, SO, or SO₂. Each J may independently be NH, NG¹, or NR. Each J may independently be G¹, CH₂, CHG¹, or CG¹₂. Each J may independently be O, CH₂, S, or NH. Each J may independently be O, CH₂, or NH. Each J may independently be O, or CH₂. Each J may independently be G¹, O, CHG¹, or NH. Each J may independently be G¹, O, or CHG¹. Each J may independently be G¹, or O. Each J may independently be O, or S. Each J may independently be G¹. Each J may independently be CH₂. Each J may be CHG¹. Each J may be CG¹₂. Each J may be NR. Each J may be SO₂. Each J may be SO. Each J may be NG¹. Each J may be NH. Each J may be S. Each J may be O.

In certain embodiments, each M may independently be H, F, Cl, Br, CH₂OH, CH₂F, CH₂Cl, CHCl₂, CCl₃, CH₂Br, CHBr₂ or CBr₃. Each M may independently be Cl, Br, CH₂Cl, CHCl₂, CCl₃, CH₂Br, CHBr₂, or CBr₃. Each M may independently be Cl, CH₂Cl, CHCl₂, or CCl₃. Each M may independently be Br, CH₂Br, CHBr₂, or CBr₃. Each M may independently be Cl, or Br. Each M may independently be CH₂F, CH₂Cl, or CH₂Br. Each M may independently be CHCl₂, or CHBr₂. Each M may independently be CCl₃, or CBr₃. Each M may independently be CH₂Cl, CHCl₂, or CCl₃. Each M may independently be CH₂Br, CHBr₂, or CBr₃. Each M may independently be Cl, CH₂Cl, or CHCl₂. Each M may independently be Br, CH₂Br, or CHBr₂. Each M may independently be CH₂Cl, or CHCl₂. Each M may independently be CH₂Br, or CHBr₂. Each M may independently be Cl, or CCl₃. Each M may independently be Br, or CBr₃. Each M may be H. Each M may be Cl. Each M may be Br. Each M may be CHCl₂. Each M may be CCl₃. Each M may be CH₂Br. Each M may be CHBr₂. Each M may be CBr₃. Each M may be CH₂Cl. Each M may be CH₂F. Each M may be CH₂OH. Each M may be F.

Each L may independently be H or A-D. Each L may be H. Each L may be A-D.

Each A may independently be O, S, NH, NG¹, N+H2, or N+HG¹. Each A may independently be O, NH, or N⁺H₂. Each A may independently be O, S, NH, or N⁺H₂. Each A may independently be O, S, or NH. Each A may independently be O, or NH. Each A may independently be O, or S. Each A may be S. Each A may be NH. Each A may be NG¹. Each A may be N⁺H₂. Each A may be N⁺HG¹.

Each D may independently be H, G¹, R, or a moiety selected from TABLE 1. Each D may independently be H, G¹, or R. Each D may independently be H, or R. Each D may independently be G¹, or R. Each D may independently be H, or G¹. Each D may independently be a moiety selected from TABLE 1.

Each L² may independently be H or A²-D². Each L² may be H. Each L² may be A²-D².

Each A² may independently be O, S, SO, SO₂, NH, NG¹, N⁺H₂, or N⁺HG¹. Each A² may independently be O, S, SO, or SO₂. Each A² may independently be O, NH, NG¹, N³⁰ H₂, or N⁺HG¹. Each A² may independently be S, SO, SO₂, NH, NG¹, N⁺H₂, or N⁺HG¹. Each A² may independently be O, S, SO, SO₂, NH, or N⁺H₂. Each A² may independently be S, SO, or SO₂. Each A² may independently be NH, NG¹, N⁺H₂, or N⁺HG¹. Each A² may independently be NH, or N⁺H₂. Each A² may independently be O, S, NH, NG¹, N⁺H₂, or N⁺HG¹. Each A² may independently be O, NH, or N⁺H₂. Each A² may independently be O, S, NH, or N⁺H₂. Each A² may independently be O, S, or NH. Each A² may independently be O, or NH. Each A² may independently be O, or S. Each A² may be S. Each A² may be SO. Each A² may be SO₂. Each A² may be NH. Each A² may be NG¹. Each A² may be N⁺H₂. Each A² may be N⁺HG¹. Each A² may be O.

Each D² may independently be H, G¹, R, or a moiety selected from TABLE 1. Each D² may independently be H, G¹, or R. Each D² may independently be H or R. Each D² may independently be G¹ or R. Each D² may independently be H or G¹. Each D² may independently be a moiety selected from TABLE 1. Each D² may be H. Each D² may be G¹. Each D² may be R.

Each Q may independently be

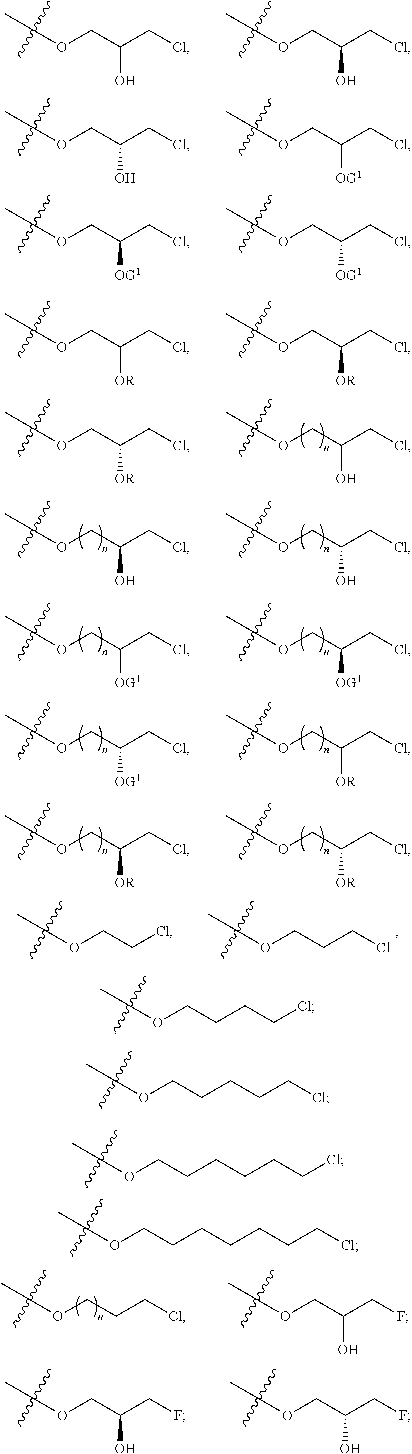

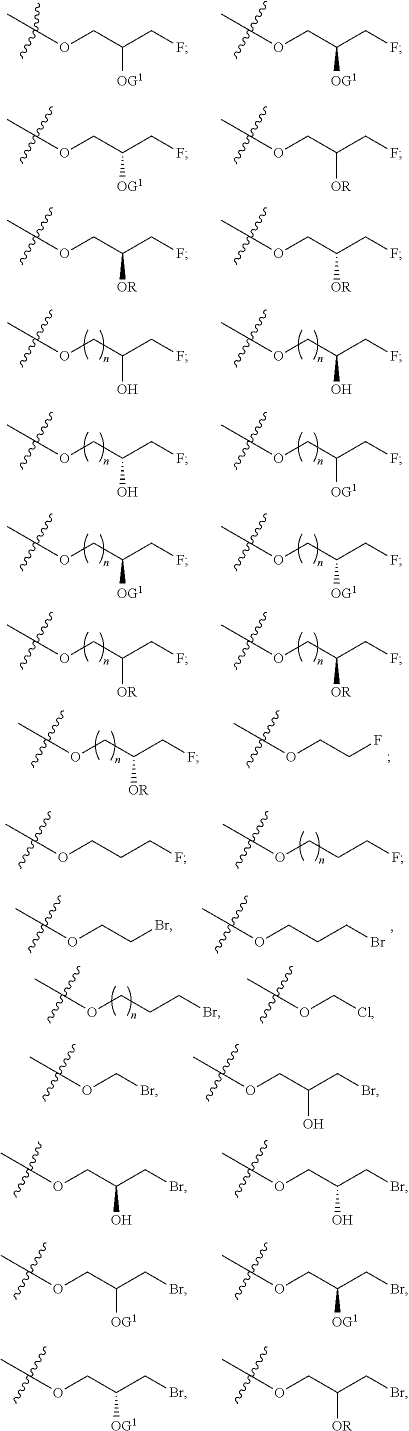

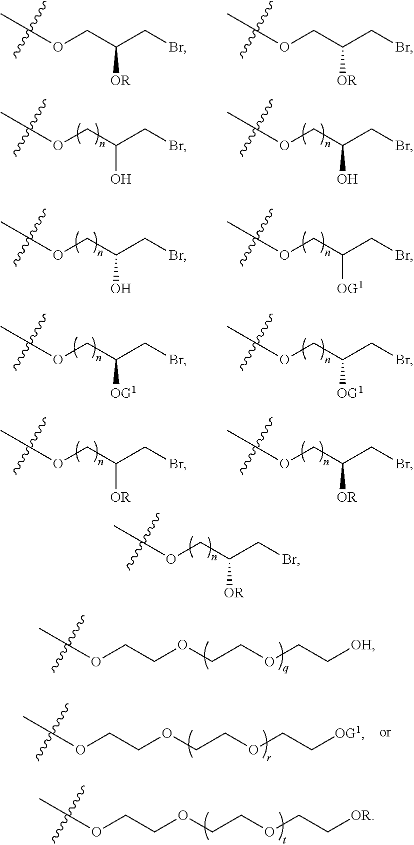

Each Q may independently be

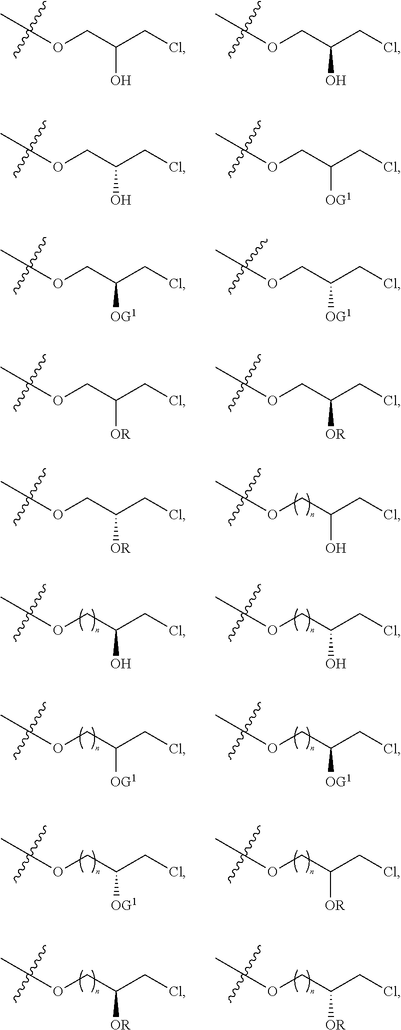

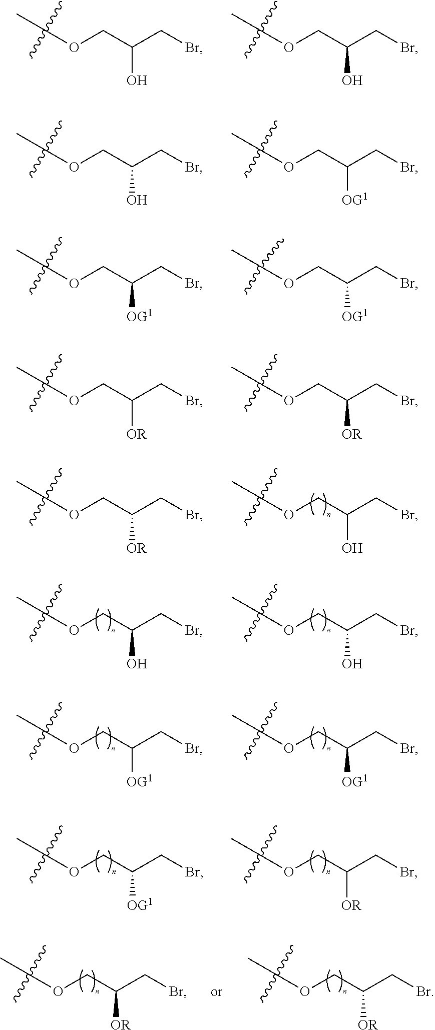

Each Q may independently be

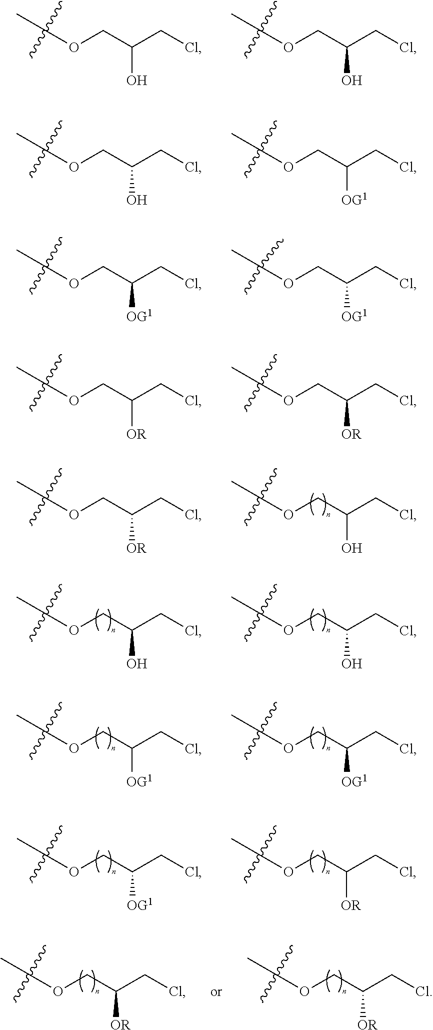

Each Q may independently be

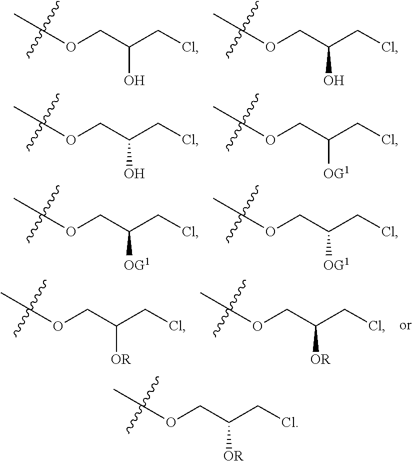

Each Q may independently be

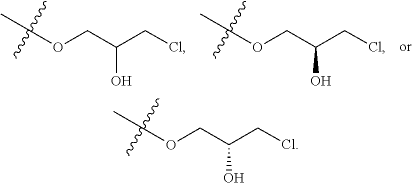 [SMILES: *OCC(O)CCl ; *OC[C@H](O)CCl ; "or" ; *OC[C@@H](O)CCl]

Each Q may independently be

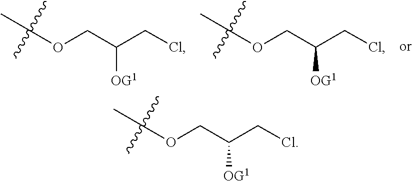

Each Q may independently be

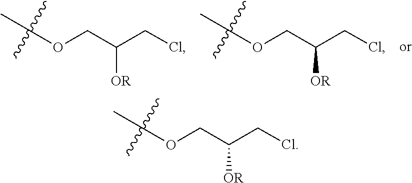

Each Q may independently be

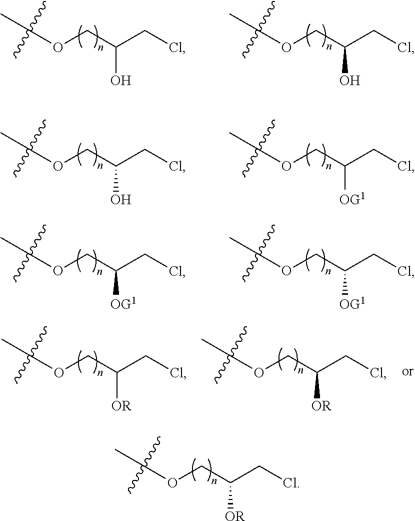

Each Q may independently be

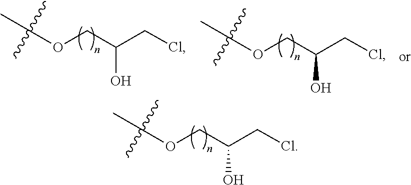

Each Q may independently be

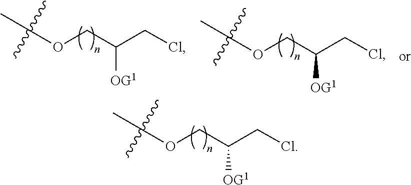

Each Q may independently be

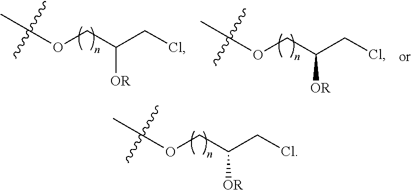

Each Q may independently be

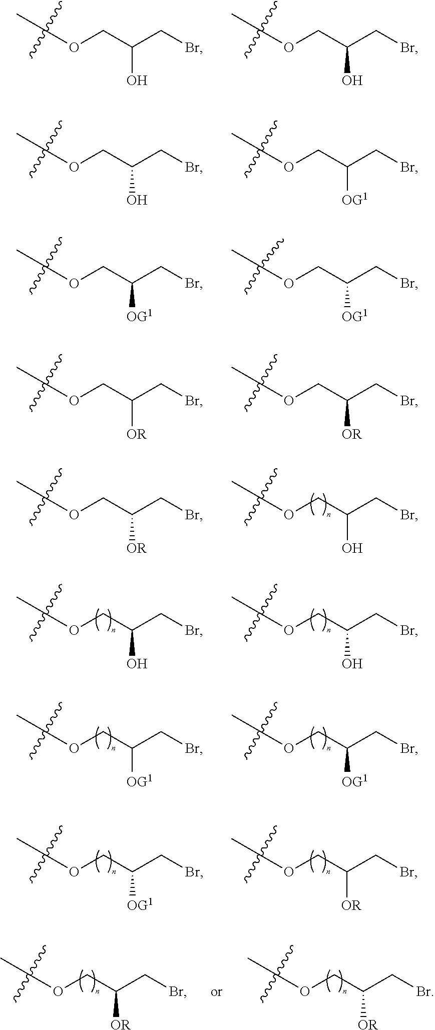

Each Q may independently be

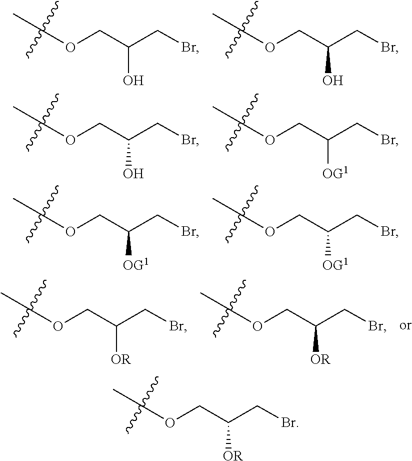

Each Q may independently be

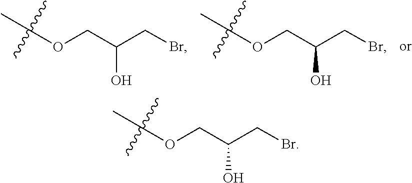 [SMILES: *OCC(O)CBr ; *OC[C@H](O)CBr ; "or" ; *OC[C@@H](O)CBr]

Each Q may independently be

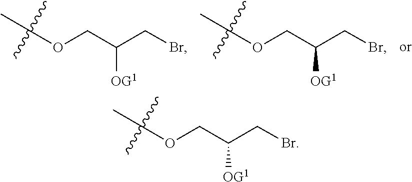

Each Q may independently be

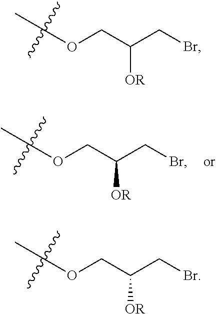

Each Q may independently be

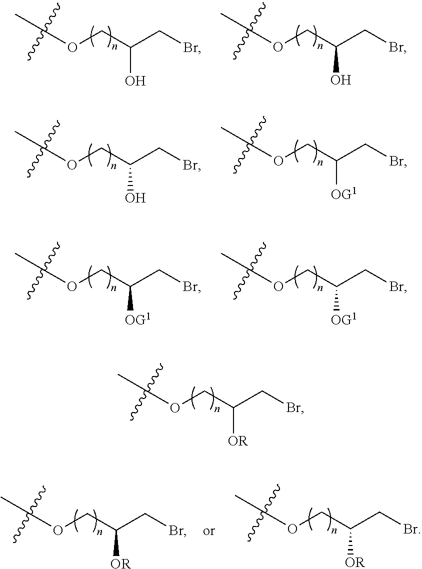

Each Q may independently be

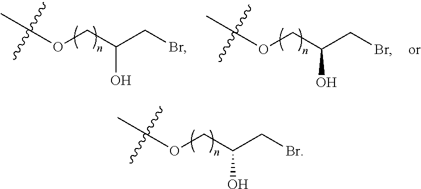

Each Q may independently be

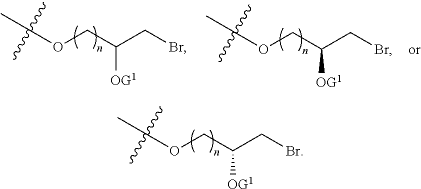

Each Q may independently be

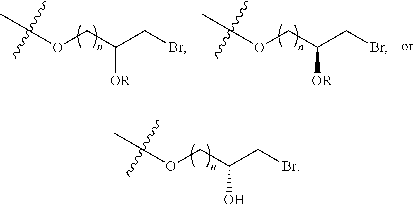

Each Q may independently be

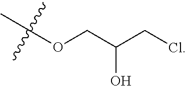 [SMILES: *OCC(O)CCl]

Each Q may independently be

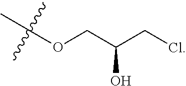 [SMILES: *OC[C@H](O)CCl]

Each Q may independently be

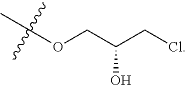 [SMILES: *OC[C@@H](O)CCl]

Each Q may independently be

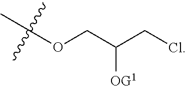

Each Q may independently be

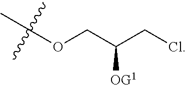

Each Q may independently be

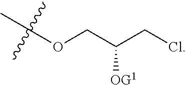

Each Q may independently be

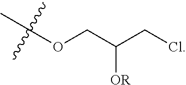

Each Q may independently be

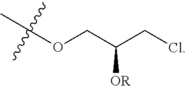

Each Q may independently be

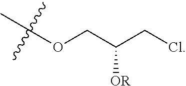

Each Q may independently be

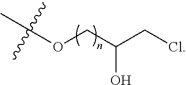

Each Q may independently be

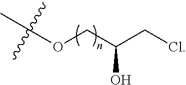

Each Q may independently be

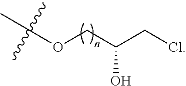

Each Q may independently be

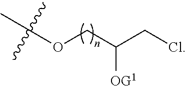

Each Q may independently be

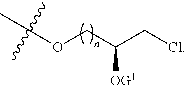

Each Q may independently be

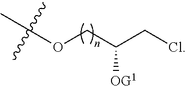

Each Q may independently be

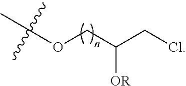

Each Q may independently be

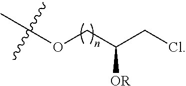

Each Q may independently be

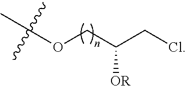

Each Q may independently be

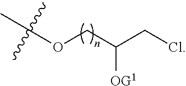

Each Q may independently be

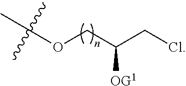

Each Q may independently be

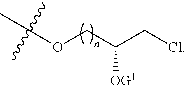

Each Q may independently be

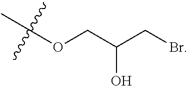 [SMILES: *OCC(O)CBr]

Each Q may independently be

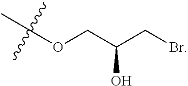 [SMILES: *OC[C@H](O)CBr]

Each Q may independently be

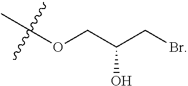 [SMILES: *OC[C@@H](O)CBr]

Each Q may independently be

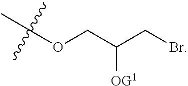

Each Q may independently be

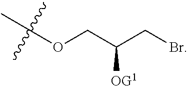

Each Q may independently be

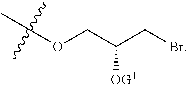

Each Q may independently be

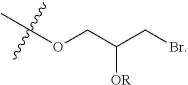

Each Q may independently be

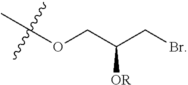

Each Q may independently be

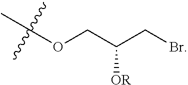

Each Q may independently be

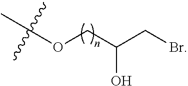

Each Q may independently be

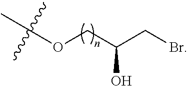

Each Q may independently be

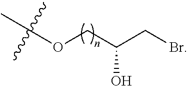

Each Q may independently be

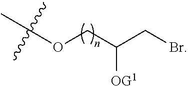

Each Q may independently be

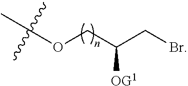

Each Q may independently be

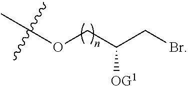

Each Q may independently be

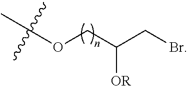

Each Q may independently be

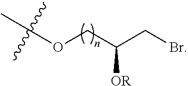

Each Q may independently be

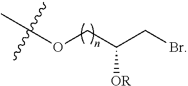

Each Q may independently be

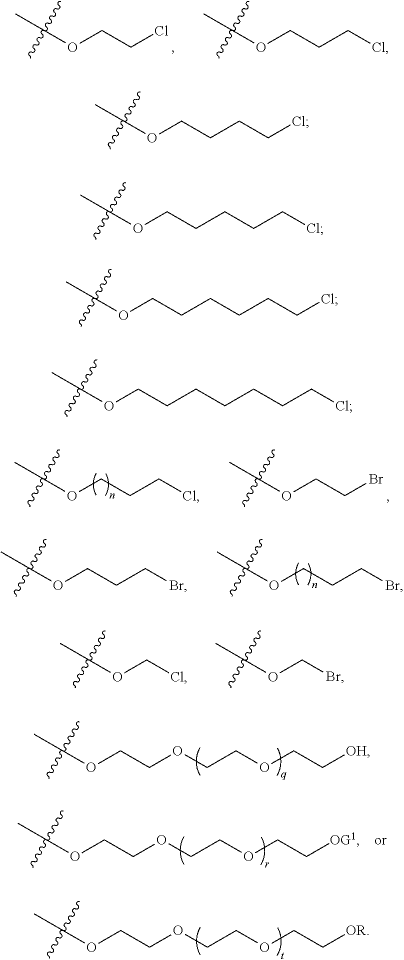

Each Q may independently be

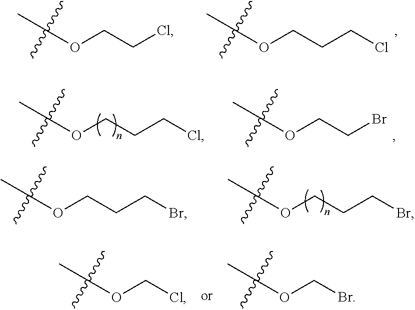

Each Q may independently be

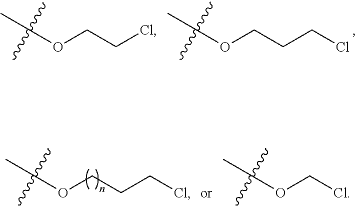

Each Q may independently be

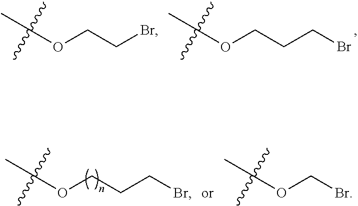

Each Q may independently be

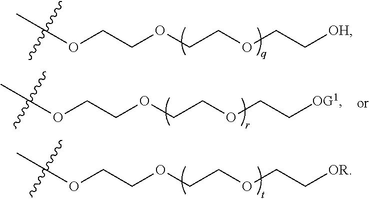

### Each Q may be

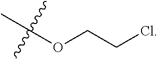 [SMILES: *OCCCl]

### Each Q may be

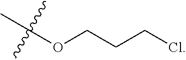 [SMILES: *OCCCCl]

Each Q may independently be

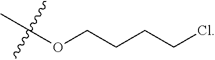 [SMILES: *OCCCCCl]

Each Q may independently be

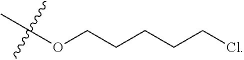 [SMILES: *OCCCCCCl]

Each Q may independently be

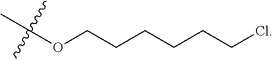 [SMILES: *OCCCCCCCl]

Each Q may independently be

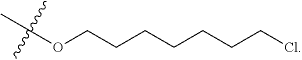 [SMILES: *OCCCCCCCCl]

Each Q may independently be

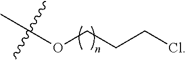

### Each Q may be

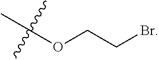 [SMILES: *OCCBr]

### Each Q may be

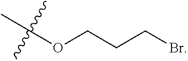 [SMILES: *OCCCBr]

Each Q may independently be

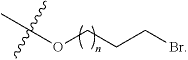

Each Q may independently be

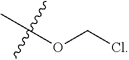 [SMILES: *OCCl]

Each Q may independently be

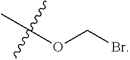 [SMILES: *OCBr]

Each Q may independently be

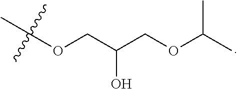 [SMILES: *OCC(O)COC(C)C]

Each Q may independently be

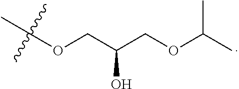 [SMILES: *OC[C@H](O)COC(C)C]

Each Q may independently be

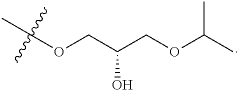 [SMILES: *OC[C@@H](O)COC(C)C]

Each Q may independently be

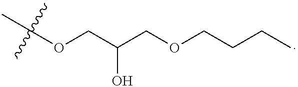 [SMILES: *OCC(O)COCCCC]

Each Q may independently be

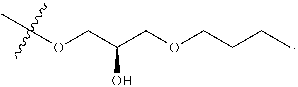 [SMILES: *OC[C@H](O)COCCCC]

Each Q may independently be

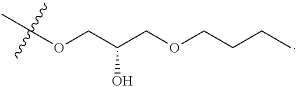 [SMILES: *OC[C@@H](O)COCCCC]

Each Q may independently be

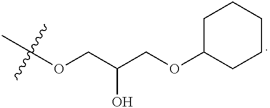 [SMILES: *OCC(O)COC1CCCCC1]

Each Q may independently be

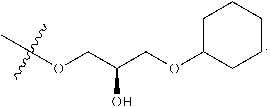 [SMILES: *OC[C@H](O)COC1CCCCC1]

Each Q may independently be

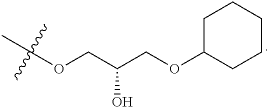 [SMILES: *OC[C@@H](O)COC1CCCCC1]

Each Q may independently be

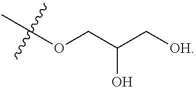 [SMILES: *OCC(O)CO]

Each Q may independently be

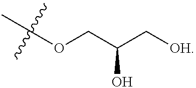 [SMILES: *OC[C@H](O)CO]

Each Q may independently be

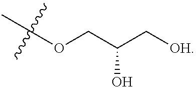 [SMILES: *OC[C@@H](O)CO]

Each Q may independently be

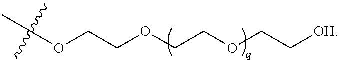

Each Q may independently be

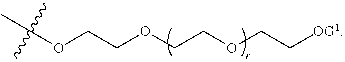

Each Q may independently be

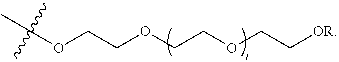

Each q may independently be 0, 1, 2, 3, 4, 5, 6 or 7. Each q may independently be 0 to 1, 0 to 2, 0 to 3, 0 to 4, 0 to 5, 0 to 6, or 0 to 7. Each q may independently be 1 to 2, 1 to 3, 1 to 4, 1 to 5, 1 to 6, or 1 to 7. Each q may independently be 2 to 3, 2 to 4, 2 to 5, 2 to 6, or 2 to 7. Each q may independently be 3 to 4, 3 to 5, 3 to 6, or 3 to 7. Each q may be 0. Each q may be 1. Each q may be 2. Each q may be 3. Each q may be 4. Each q may be 5. Each q may be 6. Each q may be 7.

Each r may independently be 0, 1, 2, 3, 4, 5, 6 or 7. Each r may independently be 0 to 1, 0 to 2, 0 to 3, 0 to 4, 0 to 5, 0 to 6, 0 to 7. Each r may independently be 1 to 2, 1 to 3, 1 to 4, 1 to 5, 1 to 6, or 1 to 7. Each r may independently be 2 to 3, 2 to 4, 2 to 5, 2 to 6, or 2 to 7. Each r may independently be 3 to 4, 3 to 5, 3 to 6, or 3 to 7. Each r may be 0. Each r may be 1. Each r may be 2. Each r may be 3. Each r may be 4. Each r may be 5. Each r may be 6. Each r may be 7.

Each t may independently be 0, 1, 2, 3, 4, 5, 6 or 7. Each t may independently be 0 to 1, 0 to 2, 0 to 3, 0 to 4, 0 to 5, 0 to 6, or 0 to 7. Each t may independently be 1 to 2, 1 to 3, 1 to 4, 1 to 5, 1 to 6, or 1 to 7. Each t may independently be 2 to 3, 2 to 4, 2 to 5, 2 to 6, or 2 to 7. Each t may independently be 3 to 4, 3 to 5, 3 to 6, or 3 to 7. Each t may be 0. Each t may be 1. Each t may be 2. Each t may be 3. Each t may be 4. Each t may be 5. Each t may be 6. Each t may be 7.

Each n may independently be 0, 1, 2, 3, 4, 5, 6, 7 or 8. Each n may independently be 0 to 1, 0 to 2, 0 to 3, 0 to 4, 0 to 5, 0 to 6, 0 to 7, or 0 to 8. Each n may independently be 1 to 2, 1 to 3, 1 to 4, 1 to 5, 1 to 6, 1 to 7, or 1 to 8. Each n may independently be 2 to 3, 2 to 4, 2 to 5, 2 to 6, 2 to 7, or 2 to 8. Each n may independently be 3 to 4, 3 to 5, 3 to 6, 3 to 7, or 3 to 8. Each n may be 0. Each n may be 1. Each n may be 2. Each n may be 3. Each n may be 4. Each n may be 5. Each n may be 6. Each n may be 7. Each n may be 8.

Each R⁸ may independently be a moiety selected from TABLE 1. Each R⁸ may independently be an amino acid based moiety or a polyethylene glycol based moiety selected from TABLE 1. Alternatively, each R⁸ may independently an amino acid based moiety selected from TABLE 1. Each R⁸ may be

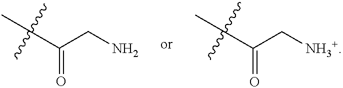

G may be linear, branched or non-aromatic cyclic, substituted or unsubstituted, saturated or unsaturated C₁-C₂₀ alkyl. G may be linear, branched or non-aromatic cyclic, substituted or unsubstituted, saturated or unsaturated C₁-C₁₉ alkyl G may be linear, branched or non-aromatic cyclic, substituted or unsubstituted, saturated or unsaturated C₁-C₁₈ alkyl. G may be linear, branched or non-aromatic cyclic, substituted or unsubstituted, saturated or unsaturated C₁-C₁₇ alkyl. G may be linear, branched or non-aromatic cyclic, substituted or unsubstituted, saturated or unsaturated C₁-C₁₆ alkyl. G may be linear, branched or non-aromatic cyclic, substituted or unsubstituted, saturated or unsaturated C₁-C₁₅ alkyl. G may be linear, branched or non-aromatic cyclic, substituted or unsubstituted, saturated or unsaturated C₁-C₁₄ alkyl. G may be linear, branched or non-aromatic cyclic, substituted or unsubstituted, saturated or unsaturated C₁-C₁₃ alkyl. G may be linear, branched or non-aromatic cyclic, substituted or unsubstituted, saturated or unsaturated C₁-C₁₂ alkyl. G may be linear, branched or non-aromatic cyclic, substituted or unsubstituted, saturated or unsaturated C₁-C₁₁ alkyl. G may be linear, branched or non-aromatic cyclic, substituted or unsubstituted, saturated or unsaturated C₁-C₁₀ alkyl. G may be linear, branched or non-aromatic cyclic, substituted or unsubstituted, saturated or unsaturated C₁-C₉ alkyl. G may be linear, branched or non-aromatic cyclic, substituted or unsubstituted, saturated or unsaturated C₁-C₈ alkyl. G may be linear, branched or non-aromatic cyclic, substituted or unsubstituted, saturated or unsaturated C₁-C₇ alkyl. G may be linear, branched or non-aromatic cyclic, substituted or unsubstituted, saturated or unsaturated C₁-C₆ alkyl. G may be linear, branched or non-aromatic cyclic, substituted or unsubstituted, saturated or unsaturated C₁-C₅ alkyl. G may be linear, branched or non-aromatic cyclic, substituted or unsubstituted, saturated or unsaturated C₁-C₄ alkyl. G may be linear, branched or non-aromatic cyclic, substituted or unsubstituted, saturated or unsaturated C₁-C₃ alkyl. G may be linear, branched or non-aromatic cyclic, substituted or unsubstituted, saturated or unsaturated C₁-C₂ alkyl. G may be substituted or unsubstituted methyl. One or more atoms of G may be optionally replaced with a heteroatom, for example oxygen. G may be alkynyl, for example propyn-3-yl (i.e., propargyl). G may be alkenyl, for example, propen-3-yl (i.e., allyl).

G¹ may be linear or branched, or aromatic cyclic or non-aromatic cyclic, substituted or unsubstituted, saturated or unsaturated C₁-C₁₀ alkyl. G¹ may be a branched, linear, or non-aromatic cyclic, substituted or unsubstituted, saturated or unsaturated C₁-C₁₀ alkyl. G¹ may be a branched, linear, or non-aromatic cyclic, substituted or saturated or unsaturated C₁-C₁₀ alkyl. G¹ may be a branched, unbranched, or aromatic cyclic or non-aromatic cyclic, substituted or unsubstituted, saturated or unsaturated C₁-C₆ alkyl. G¹ may be a branched, unbranched, or aromatic cyclic or non-aromatic cyclic, substituted or unsubstituted, saturated or unsaturated C₁-C₈ alkyl. G¹ may be a branched, unbranched, or aromatic cyclic or non-aromatic cyclic, substituted or unsubstituted, saturated or unsaturated C₁-C₇ alkyl. G¹ may be a branched, unbranched, or aromatic cyclic or non-aromatic cyclic, substituted or unsubstituted, saturated or unsaturated C₁-C₆ alkyl. G¹ may be a branched, unbranched, or aromatic cyclic or non-aromatic cyclic, substituted or unsubstituted, saturated or unsaturated C₁-C₅ alkyl. G¹ may be a branched, unbranched, or non-aromatic cyclic, substituted or unsubstituted, saturated or unsaturated C₁-C₄ alkyl. G¹ may be a branched, unbranched, or non-aromatic cyclic, substituted or unsubstituted, saturated or unsaturated C₁-C₃ alkyl. G¹ may be a branched, unbranched, or non-aromatic cyclic, substituted or unsubstituted, saturated or unsaturated C₁-C₂ alkyl. G¹ may be substituted or unsubstituted methyl.

An optional substituent for any of the C₁-C₂₀ alkyl, C₁-C₁₀ alkyl or cyclic C₃-C₁₀ alkyl moieties may be selected from the group consisting of oxo, OR⁸, COOH, R⁹, OH, OR⁹, F, Cl, Br, I, NH₂, NHR⁹, NR⁹₂, CN, SH, SR⁹, SO₃H, SO₃R⁹, SO₂R⁹, OSO₃R⁹, OR, CO₂R⁹, CONH₂, CONHR⁹, CONHR, CONR⁹₂, NHR, OPO₃H₃, CONR⁹R, NR⁹R, and NO₂. An optional substituent may be selected from the group consisting of: oxo (i.e., ═O), OR⁸, COOH, R⁹, OH, OR⁹, F, Cl, Br, I, NH₂, NHR⁹, NR⁹₂, CN, SH, SR⁹, SO₃H, SO₃R⁹, SO₂R⁹, OSO₃R⁹, and NO₂. An optional substituent may be selected from the group consisting of: oxo (i.e., ═O), OR⁸, COOH, R⁹, OH, OR⁹, F, Cl, Br, I, NH₂, NHR⁹, NR⁹₂, SO₃H, SO₃R⁹, SO₂R⁹, and NO₂. An optional substituent may be selected from the group consisting of: oxo (i.e., ═O), OR⁸, COOH, R⁹, OH, OR⁹, F, Cl, Br, I, NH₂, and NO₂. An optional substituent may be selected from the group consisting of: oxo (i.e., ═O), OR⁸, COOH, R⁹, OH, OR⁹, F, Cl, Br, and I. An optional substituent may be selected from the group consisting of: oxo (i.e., ═O), OR⁸, COOH, OH, F, Cl, Br, and I. An optional substituent may be selected from the group consisting of: oxo (i.e., ═O), OR⁸, COOH, OH, F, and Cl. Each C₁-C₁₀ alkyl or cyclic C₃-C₁₀ alkyl may be substituted with, for example, 1, 2, 3, 4, 5, or 6 substituents.

Each R⁹ may independently be unsubstituted C₁-C₁₀ alkyl. Each R⁹ may independently be unsubstituted C₁-C₉ alkyl. Each R⁹ may independently be unsubstituted C₁-C₈ alkyl. Each R⁹ may independently be unsubstituted C₁-C₇ alkyl. Each R⁹ may independently be unsubstituted C₁-C₆ alkyl. Each R⁹ may independently be unsubstituted C₁-C₅ alkyl. Each R⁹ may independently be unsubstituted C₁-C₄ alkyl. Each R⁹ may independently be unsubstituted C₁-C₃ alkyl. Each R⁹ may independently be unsubstituted C₁-C₂ alkyl. Each R⁹ may independently be unsubstituted C₁ alkyl. Each R⁹ may independently be unsubstituted C₂ alkyl. Each R⁹ may independently be unsubstituted C₃ alkyl. Each R⁹ may independently be unsubstituted C₄ alkyl. Each R⁹ may independently be unsubstituted C₅ alkyl. Each R⁹ may independently be unsubstituted C₆ alkyl. Each R⁹ may independently be unsubstituted C₇ alkyl. Each R⁹ may independently be unsubstituted C₈ alkyl. Each R⁹ may independently be unsubstituted C₉ alkyl. Each R⁹ may independently be unsubstituted C₁₀ alkyl.

Each R may independently be C₁-C₁₀ acyl. Each R may independently be C₁-C₉ acyl. Each R may independently be C₁-C₈ acyl. Each R may independently be C₁-C₇ acyl. Each R may independently be C₁-C₆ acyl. Each R may independently be C₁-C₅ acyl. Each R may independently be C₁-C₄ acyl. Each R may independently be C₁-C₃ acyl. Each R may independently be C₁-C₂ acyl. Each R may independently be C₁ acyl. Each R may independently be C₂ acyl. Each R may independently be C₃ acyl. Each R may independently be C₄ acyl. Each R may independently be C₅ acyl. Each R may independently be C₆ acyl. Each R may independently be C₇ acyl. Each R may independently be C₈ acyl. Each R may independently be C₉ acyl. Each R may independently be C₁₀ acyl.

Each of R¹ and R² may independently be hydrogen or linear or branched, substituted or unsubstituted, saturated or unsaturated C₁-C₁₀ alkyl, or R¹ and R² together may form a substituted or unsubstituted, saturated or unsaturated cyclic C₃-C₁₀ alkyl. Each of R¹ and R² may independently be branched or unbranched, substituted or unsubstituted, saturated or unsaturated C₁-C₁₀ alkyl. Each of R¹ and R² may independently be branched or unbranched, substituted or unsubstituted, saturated or unsaturated C₁-C₉ alkyl. Each of R¹ and R² may independently be branched or unbranched, substituted or unsubstituted, saturated or unsaturated C₁-C₈ alkyl. Each of R¹ and R² may independently be branched or unbranched, substituted or unsubstituted, saturated or unsaturated C₁-C₇ alkyl. Each of R¹ and R² may independently be branched or unbranched, substituted or unsubstituted, saturated or unsaturated C₁-C₆ alkyl. Each of R¹ and R² may independently be branched or unbranched, substituted or unsubstituted, saturated or unsaturated C₁-C₅ alkyl. Each of R¹ and R² may independently be branched or unbranched, substituted or unsubstituted, saturated or unsaturated C₁-C₄ alkyl. Each of R¹ and R² may independently be branched or unbranched, substituted or unsubstituted, saturated or unsaturated C₁-C₃ alkyl. Each of R¹ and R² may independently be branched or unbranched, substituted or unsubstituted, saturated or unsaturated C₁-C₂ alkyl, for example each of R¹ and R² may be methyl. Each of R¹ and R² may be CH₃. R¹ and R² together may form a substituted or unsubstituted, saturated or unsaturated cyclic C₃-C₁₀ alkyl. R¹ and R² together may form an unsubstituted, saturated cyclic C₆ alkyl. Each of R¹ and R² may be hydrogen.

R³, R⁴, R⁵ and R⁶ may each independently be H, halo or linear or branched, substituted or unsubstituted, saturated or unsaturated C₁-C₁₀ alkyl. R³, R⁴, R⁵ and R⁶ may each be H. R³, R⁴, R⁵ and R⁶ may each be halo. R³, R⁴, R⁵ and R⁶ may each be F. R³, R⁴, R⁵ and R⁶ may each be Cl. R³, R⁴, R⁵ and R⁶ may each be Br. R³, R⁴, R⁵ and R⁶ may each be I. R³, R⁴, R⁵ and R⁶ may each be methyl. At least one of R³, R⁴, R⁵ and R⁶ may independently be H. At least one of R³, R⁴, R⁵ and R⁶ may independently be halo. At least one of R³, R⁴, R⁵ and R⁶ may independently be I. At least one of R³, R⁴, R⁵ and R⁶ may independently be Cl. At least one of R³, R⁴, R⁵ and R⁶ may independently be Br. At least one of R³, R⁴, R⁵ and R⁶ may be I. At least one of R³, R⁴, R⁵ and R⁶ may independently be methyl.

The compounds described herein are meant to include all racemic mixtures and all individual enantiomers or combinations thereof, whether or not they are specifically depicted herein. Alternatively, one or more of the OH groups on the above compounds may be substituted to replace the H with a moiety selected from TABLE 1.

In yet other embodiments, the present disclosure provide the use of any of the compounds disclosed herein for modulating androgen receptor (AR) activity. For example, in certain embodiments modulating androgen receptor (AR) activity is in a mammalian cell.

In other examples, modulating androgen receptor (AR) activity is for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. For example, in certain embodiments the indication is prostate cancer, for example, castration resistant prostate cancer. In other examples, the prostate cancer is androgen-dependent prostate cancer. In other further embodiments, the spinal and bulbar muscular atrophy is Kennedy's disease.

The present disclosure also provides a method of modulating androgen receptor (AR) activity, the method comprising administering any of the compounds disclosed herein, or pharmaceutically acceptable salt thereof, to a subject in need thereof. For example, in certain specific embodiments modulating androgen receptor (AR) activity is for the treatment of one or more of the following: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In certain embodiments, the spinal and bulbar muscular atrophy is Kennedy's disease.

The present disclosure also provides a pharmaceutical composition comprising any one or more of the compounds disclosed herein and a pharmaceutically acceptable carrier. The pharmaceutical composition may be for treating one or more of the following: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration.

In accordance with another embodiment, there is provided a use of the compounds as described anywhere herein for preparation of a medicament for modulating androgen receptor (AR).

In accordance with a further embodiment, there is provided a method of screening for androgen receptor modulating compounds, wherein the compounds screened are selected from the compounds as described anywhere herein.

The modulating of the androgen receptor (AR) activity may be in a mammalian cell. The modulating of the androgen receptor (AR) activity may be in a mammal. The mammal may be a human.

Alternatively, the administering may be to a mammal. The administering may be to a mammal in need thereof and in an effective amount for the treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy (e.g., Kennedy's disease), and age-related macular degeneration.

The mammalian cell may be a human cell. The modulating AR activity may be for inhibiting AR N-terminal domain activity. The modulating AR activity may be for inhibiting AR activity. The modulating may be in vivo. The modulating AR activity may be for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy (e.g., Kennedy's disease), and age-related macular degeneration. The indication may be prostate cancer. The prostate cancer may be castration-resistant prostate cancer. The prostate cancer may be androgen-dependent prostate cancer.

In another embodiment, the disclosure provides a pharmaceutical composition comprising a compound of Formula I, an additional therapeutic agent and a pharmaceutically acceptable carrier. For example, in some embodiments the additional therapeutic agent is for treating prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy or age-related macular degeneration.

In other examples, the additional therapeutic agent is MDV3100, TOK 001, TOK 001; ARN-509; abiraterone, bicalutamide, nilutamide, flutamide, cyproterone acetate, docetaxel, Bevacizumab (Avastin), OSU-HDAC42, VITAXIN, sunitumib, ZD-4054, VN/124-1. Cabazitaxel (XRP-6258), MDX-010 (Ipilimurnab), OGX 427, OGX 011, finasteride, dutasteride, turosteride, bexlosteride, izonsteride, FCE 28260, SKF105,111 or related compounds thereof.

The present disclosure also provide for the use of the disclosed pharmaceutical compositions for modulating androgen receptor (AR) activity. The compositions may comprise a compound of Formula I or a compound of Formula I in combination with an additional therapeutic agent. For example, the use may be for modulating androgen receptor (AR) activity is in a mammalian cell, and modulating androgen receptor (AR) activity may be for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In certain embodiments, the indication is prostate cancer, and in other embodiments, the prostate cancer is castration resistant prostate cancer or androgen-dependent prostate cancer. In yet other embodiments, the indication is Kennedy's disease.

Also provided is a method of modulating androgen receptor (AR) activity, the method comprising administering a pharmaceutical composition comprising a compound of Formula I or a compound of Formula I in combination with an additional therapeutic agent to a subject in need thereof. In some further examples, modulating androgen receptor (AR) activity is for the treatment of one or more of the following: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. For example, the indication may be Kennedy's disease or the indication may be prostate cancer. In some specific examples, the prostate cancer is castration resistant prostate cancer, and in other examples the prostate cancer is androgen-dependent prostate cancer.

TABLE 1
Amino Acid, Polyethylene Glycol, and Phosphate Based Moieties
MOIETIES
Amino Acid Based Moieties 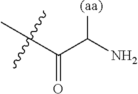
(aa) = any naturally occurring amino acid side chain

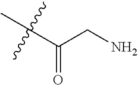 [SMILES: *C(=O)CN]

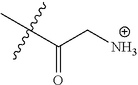 [SMILES: *C(=O)C[NH3+]]

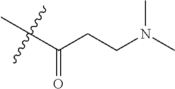 [SMILES: *C(=O)CCN(C)C]

Polyethylene Glycol Based Moieties

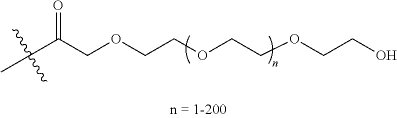

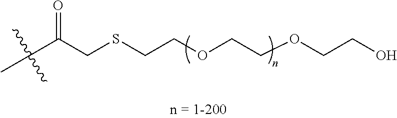

Phosphate Based Moieties

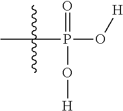 [SMILES: *P(=O)(O)O]

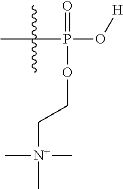 [SMILES: *P(=O)(O)OCC[N+](C)(C)C]

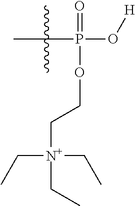 [SMILES: *P(=O)(O)OCC[N+](CC)(CC)CC]

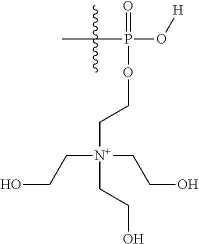 [SMILES: *P(=O)(O)OCC[N+](CCO)(CCO)CCO]

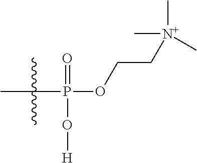 [SMILES: *P(=O)(O)OCC[N+](C)(C)C]

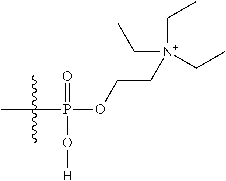 [SMILES: *P(=O)(O)OCC[N+](CC)(CC)CC]

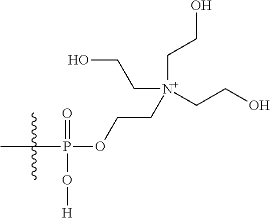 [SMILES: *P(=O)(O)OCC[N+](CCO)(CCO)CCO]

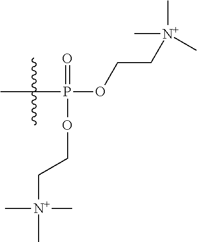

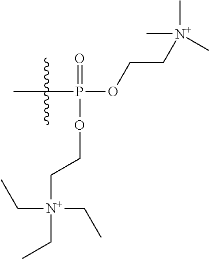 [SMILES: *P(=O)(OCC[N+](C)(C)C)OCC[N+](CC)(CC)CC]

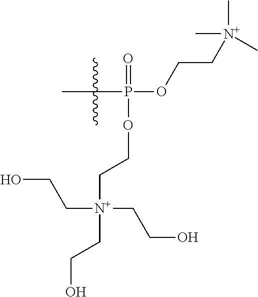 [SMILES: *P(=O)(OCC[N+](C)(C)C)OCC[N+](CCO)(CCO)CCO]

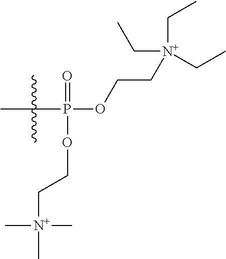 [SMILES: *P(=O)(OCC[N+](C)(C)C)OCC[N+](CC)(CC)CC]

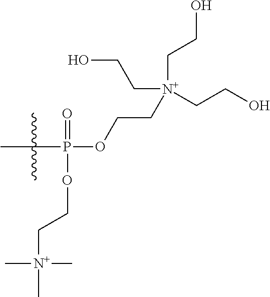 [SMILES: *P(=O)(OCC[N+](C)(C)C)OCC[N+](CCO)(CCO)CCO]

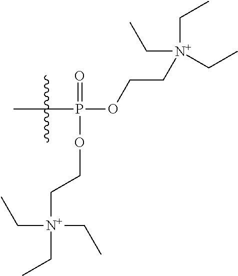 [SMILES: *P(=O)(OCC[N+](CC)(CC)CC)OCC[N+](CC)(CC)CC]

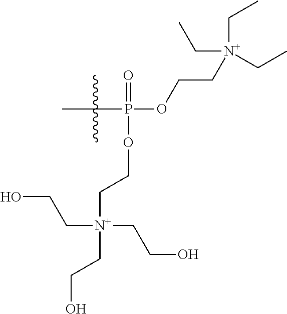 [SMILES: *P(=O)(OCC[N+](CC)(CC)CC)OCC[N+](CCO)(CCO)CCO]

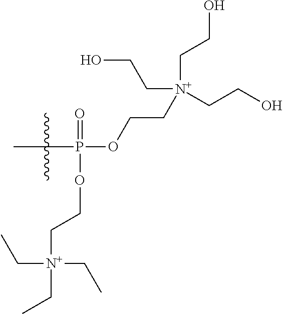 [SMILES: *P(=O)(OCC[N+](CC)(CC)CC)OCC[N+](CCO)(CCO)CCO]

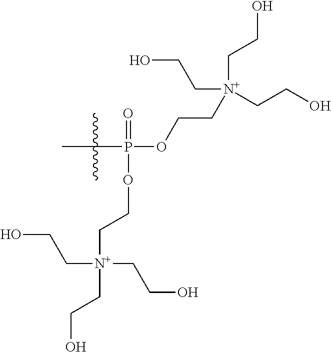 [SMILES: *P(=O)(OCC[N+](CCO)(CCO)CCO)OCC[N+](CCO)(CCO)CCO]

Moieties from TABLE 1 may be, for example, and without limitation, subdivided into three groups: 1) amino acid based moieties; 2) polyethylene glycol based moieties; and 3) phosphate based moieties. In the Moieties Table 1 above, the first four moieties are amino acid based moieties, the fifth and sixth are polyethylene glycol based moieties and the remaining moieties are phosphate based moieties.

The amino acid side chains of naturally occurring amino acids (as often denoted herein using “(aa)”) are well known to a person of skill in the art and may be found in a variety of text books such as “Molecular Cell Biology” by James Darnell et al. Third Edition, published by Scientific American Books in 1995. Often the naturally occurring amino acids are represented by the formula (NH₂)C(COOH)(H)(R), where the chemical groups in brackets are each bonded to the carbon not in brackets. R represents the side chains in this particular formula.

Those skilled in the art will appreciate that the point of covalent attachment of the moiety to the compounds as described herein may be, for example, and without limitation, cleaved under specified conditions. Specified conditions may include, for example, and without limitation, in vivo enzymatic or non-enzymatic means. Cleavage of the moiety may occur, for example, and without limitation, spontaneously, or it may be catalyzed, induced by another agent, or a change in a physical parameter or environmental parameter, for example, an enzyme, light, acid, temperature or pH. The moiety may be, for example, and without limitation, a protecting group that acts to mask a functional group, a group that acts as a substrate for one or more active or passive transport mechanisms, or a group that acts to impart or enhance a property of the compound, for example, solubility, bioavailability or localization.

In other particular embodiments of the compounds as described anywhere herein, the following compounds in Table 2 are provided.

TABLE 2
Representative Compounds

| No. | Structure | Name |
|---|---|---|
| 3 | 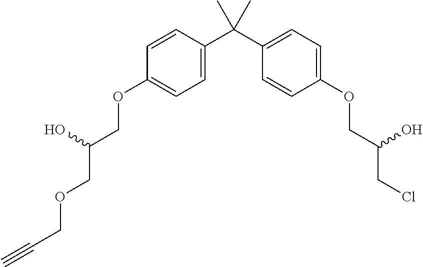 [SMILES: C#CCOCC(O)COc1ccc(C(C)(C)c2ccc(OCC(O)CCl)cc2)cc1] | 1-chloro-3-(4-(2-(4-(2-hydroxy-3- (prop-2- ynyloxy)propoxy)phenyl)propan-2- yl)phenoxy)propan-2-ol |
| 5 | 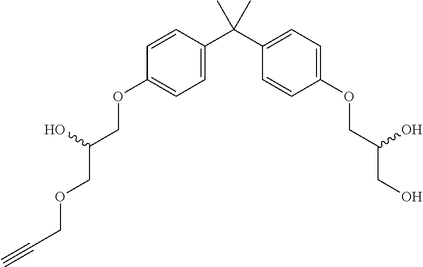 [SMILES: C#CCOCC(O)COc1ccc(C(C)(C)c2ccc(OCC(O)CO)cc2)cc1] | 3-(4-(2-(4-(2-hydroxy-3-(prop-2- ynyloxy)propoxy)phenyl)propan-2- yl)phenoxy)propane-1,2-diol |
| 10 | 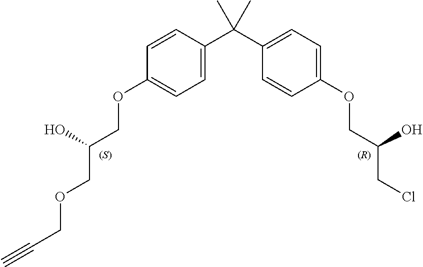 | (R)-1-chloro-3-(4-(2-(4-((S)-2-hydroxy- 3-(prop-2- ynyloxy)propoxy)phenyl)propan-2- yl)phenoxy)propan-2-ol |
| 12 | 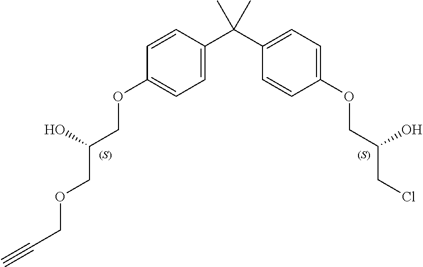 | (S)-1-chloro-3-(4-(2-(4-((S)-2-hydroxy- 3-(prop-2- ynyloxy)propoxy)phenyl)propan-2- yl)phenoxy)propan-2-ol |
| 16 | 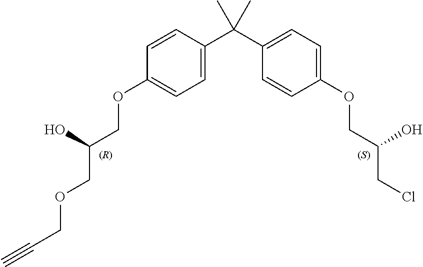 | (S)-1-chloro-3-(4-(2-(4-((R)-2-hydroxy- 3-(prop-2- ynyloxy)propoxy)phenyl)propan-2- yl)phenoxy)propan-2-ol |
| 18 | 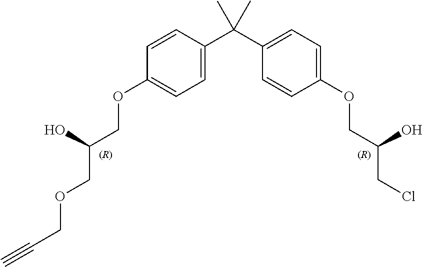 | (R)-1-chloro-3-(4-(2-(4-((R)-2- hydroxy-3-(prop-2- ynyloxy)propoxy)phenyl)propan-2- yl)phenoxy)propan-2-ol |
| 19 | 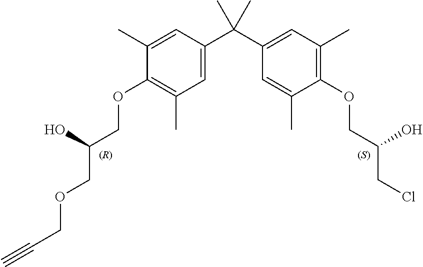 | (S)-1-chloro-3-(4-(2-(4-((R)-2-hydroxy- 3-(prop-2-ynyloxy)propoxy)-3,5- dimethylphenyl)propan-2-yl)-2,6- dimethylphenoxy)propan-2-ol |
| 20 | 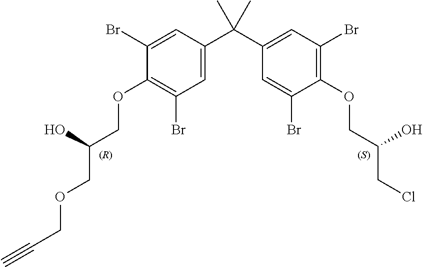 | (S)-1-chloro-3-(2,6-dibromo-4-(2-(3,5- dibromo-4-((R)-2-hydroxy-3-(prop-2- ynyloxy)propoxy)phenyl)propan-2- yl)phenoxy)propan-2-ol |
| 21 | 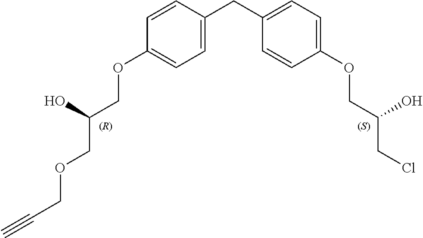 | (S)-1-chloro-3-(4-(4-((R)-2- hydroxy-3-(prop-2- ynyloxy)propoxy)benzyl)phenoxy) propan-2-ol |
| 22 | 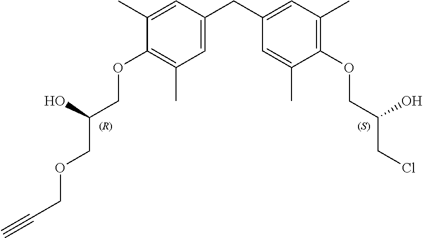 | (S)-1-chloro-3-(4-(4-((R)-2-hydroxy-3- (prop-2-ynyloxy)propoxy)-3,5- dimethylbenzyl)-2,6-dimethylphenoxy) propan-2-ol |
| 23 | 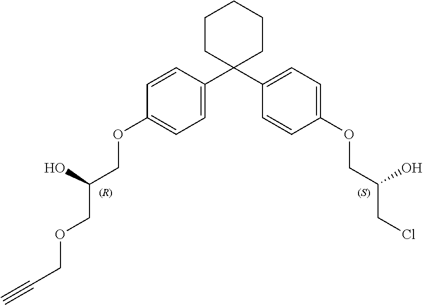 | (S)-1-chloro-3-(4-(1-(4-((R)-2- hydroxy-3-(prop-2- ynyloxy)propoxy)phenyl)cyclohexyl) phenoxy)propan-2-ol |
| 24 | 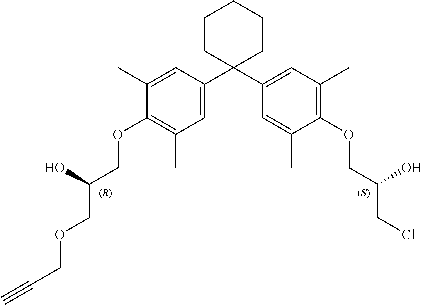 | (S)-1-chloro-3-(4-(1-(4-((R)-2-hydroxy- 3-(prop-2-ynyloxy)propoxy)-3,5- dimethylphenyl)cyclohexyl)-2,6- dimethylphenoxy)propan-2-ol |
| 25 | 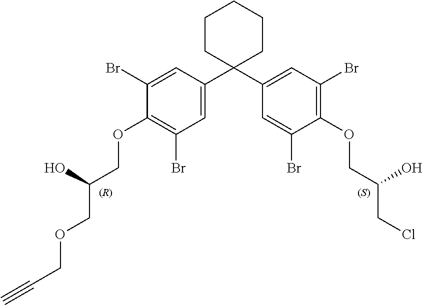 | (S)-1-chloro-3-(2,6-dibromo-4-(1- (3,5-dibromo-4-((R)-2-hydroxy-3- (prop-2-ynyloxy)propoxy)phenyl) cyclohexyl)phenoxy)propan-2-ol |
| 26 | 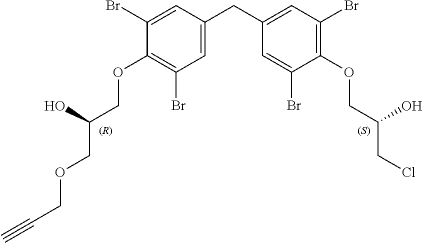 | (S)-1-chloro-3-(2,6-dibromo-4- (3,5-dibromo-4-((R)-2-hydroxy-3- (prop-2- ynyloxy)propoxy)benzyl)phenoxy) propan-2-ol |
| 27 | 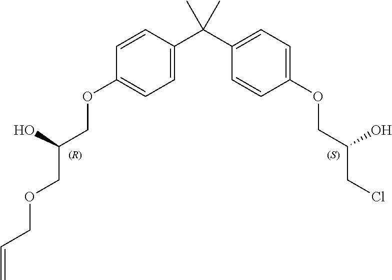 | (R)-1-(allyloxy)-3-(4-(2-(4-((S)-3- chloro-2- hydroxypropoxy)phenyl)propan-2- yl)phenoxy)propan-2-ol |
| 28 | 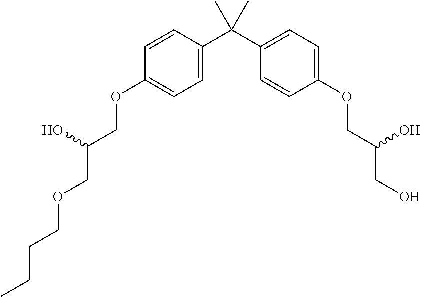 [SMILES: CCCCOCC(O)COc1ccc(C(C)(C)c2ccc(OCC(O)CO)cc2)cc1] | 3-(4-(2-(4-(3-butoxy-2- hydroxypropoxy)phenyl)propan-2- yl)phenoxy)propane-1,2-diol |
| 30 | 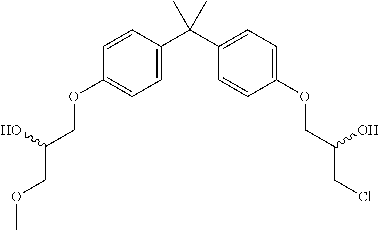 [SMILES: COCC(O)COc1ccc(C(C)(C)c2ccc(OCC(O)CCl)cc2)cc1] | 1-chloro-3-(4-(2-(4-(2-hydroxy-3- methoxypropoxy)phenyl)propan-2- yl)phenoxy)propan-2-ol |
| 31 | 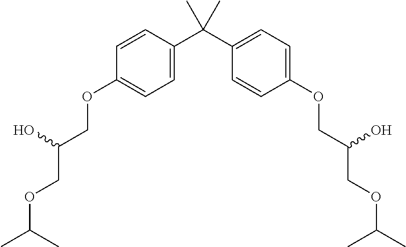 [SMILES: CC(C)OCC(O)COc1ccc(C(C)(C)c2ccc(OCC(O)COC(C)C)cc2)cc1] | 3,3′-(4,4′-(propane-2,2-diyl) bis(4,1-phenylene))bis(oxy)bis(1- isopropoxypropan-2-ol) |
| 33 | 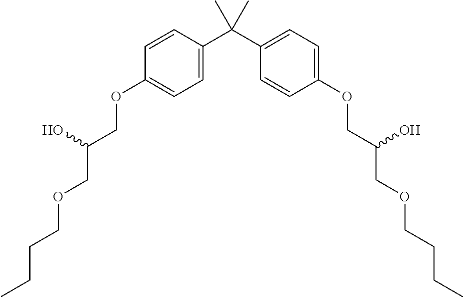 [SMILES: CCCCOCC(O)COc1ccc(C(C)(C)c2ccc(OCC(O)COCCCC)cc2)cc1] | 3,3′-(4,4′-(propane-2,2-diyl)bis(4,1- phenylene))bis(oxy)bis (1-butoxypropan-2-ol) |
| 34 | 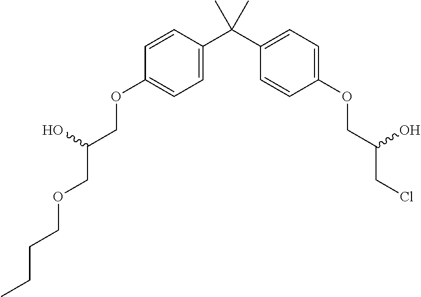 [SMILES: CCCCOCC(O)COc1ccc(C(C)(C)c2ccc(OCC(O)CCl)cc2)cc1] | 1-butoxy-3-(4-(2-(4-(3-chloro-2- hydroxypropoxy)phenyl)propan-2- yl)phenoxy)propan-2-ol |
| 35 | 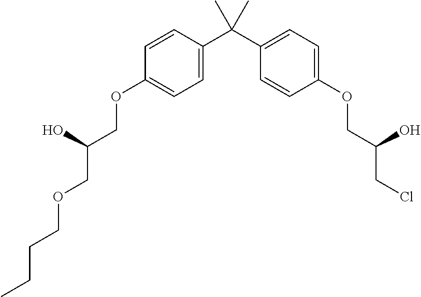 [SMILES: CCCCOC[C@@H](O)COc1ccc(C(C)(C)c2ccc(OC[C@@H](O)CCl)cc2)cc1] | (R)-1-butoxy-3-(4-(2-(4-((R)-3-chloro- 2-hydroxypropoxy)phenyl)propan-2- yl)phenoxy)propan-2-ol |
| 36 | 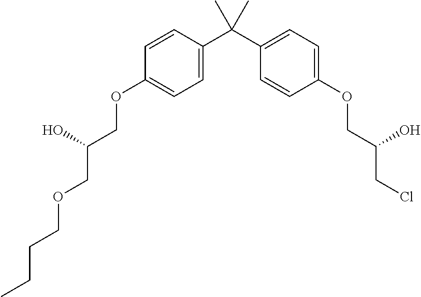 [SMILES: CCCCOC[C@H](O)COc1ccc(C(C)(C)c2ccc(OC[C@H](O)CCl)cc2)cc1] | (S)-1-butoxy-3-(4-(2-(4-((S)-3-chloro- 2-hydroxypropoxy)phenyl)propan-2- yl)phenoxy)propan-2-ol |
| 37 | 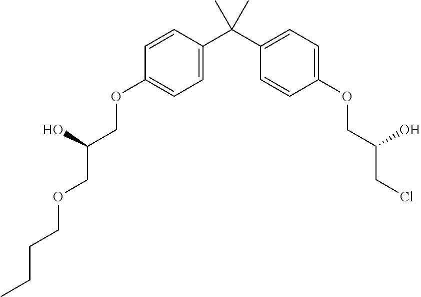 [SMILES: CCCCOC[C@@H](O)COc1ccc(C(C)(C)c2ccc(OC[C@H](O)CCl)cc2)cc1] | (R)-1-butoxy-3-(4-(2-(4-((S)-3-chloro- 2-hydroxypropoxy)phenyl)propan-2- yl)phenoxy)propan-2-ol |
| 38 | 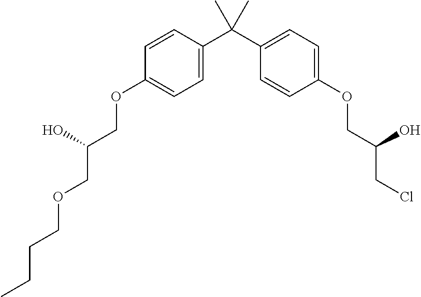 [SMILES: CCCCOC[C@H](O)COc1ccc(C(C)(C)c2ccc(OC[C@@H](O)CCl)cc2)cc1] | (S)-1-butoxy-3-(4-(2-(4-((R)-3-chloro- 2-hydroxypropoxy)phenyl)propan-2- yl)phenoxy)propan-2-ol |
| 40 | 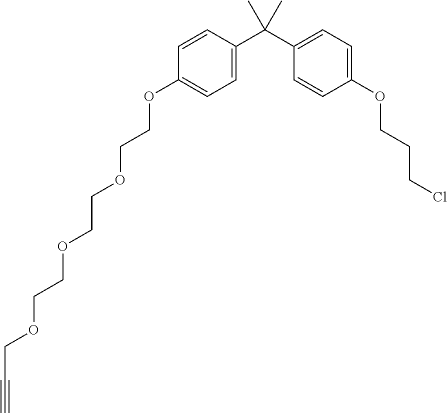 [SMILES: C#CCOCCOCCOCCOc1ccc(C(C)(C)c2ccc(OCCCCl)cc2)cc1] | 1-(3-chloropropoxy)-4-(2-(4- (2-(2-(2-(prop-2-ynyloxy)ethoxy) ethoxy)ethoxy)phenyl) propan-2-yl)benzene |
| 43 | 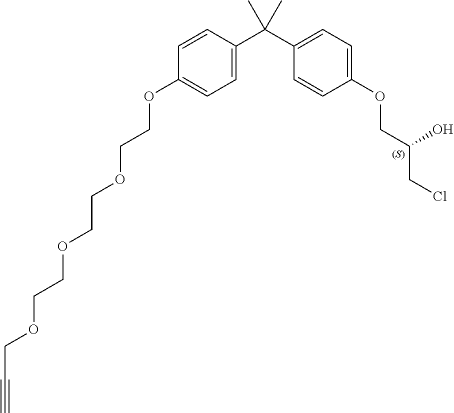 | (S)-1-chloro-3-(4-(2-(4- (2-(2-(2-(prop-2- ynyloxy)ethoxy)ethoxy)ethoxy) phenyl)propan-2-yl)phenoxy) propan-2-ol |
| 44 | 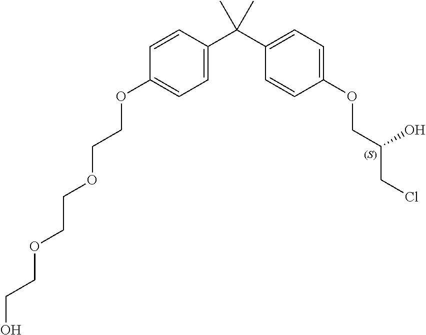 | (S)-1-chloro-3-(4-(2-(4-(2-(2-(2- hydroxyethoxy)ethoxy)ethoxy)phenyl) propan-2-yl)phenoxy)propan-2-ol |
| 46 | 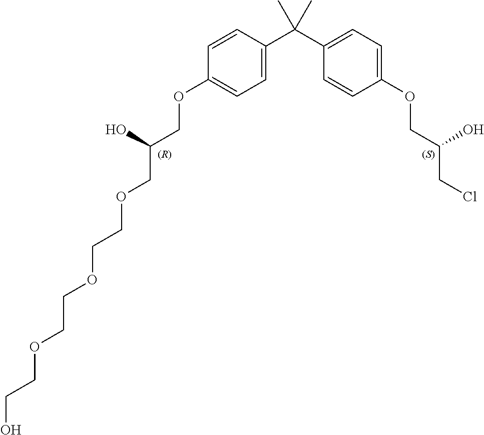 | (S)-1-chloro-3-(4-(2-(4-((R)-2- hydroxy-3-(2-(2-(2- hydroxyethoxy)ethoxy)ethoxy)propoxy) phenyl)propan-2-yl) phenoxy)propan-2-ol |
| 47 | 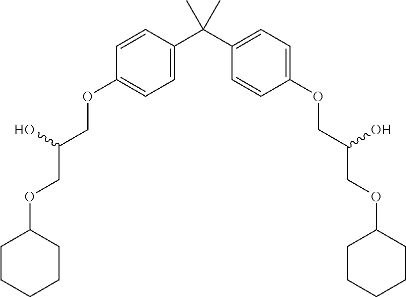 [SMILES: CC(C)(c1ccc(OCC(O)COC2CCCCC2)cc1)c1ccc(OCC(O)COC2CCCCC2)cc1] | 3,3′-(4,4′-(propane-2,2-diyl)bis(4,1- phenylene))bis(oxy)bis(1- (cyclohexyloxy)propan-2-ol) |
| 48 | 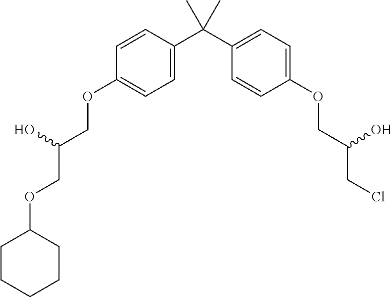 [SMILES: CC(C)(c1ccc(OCC(O)CCl)cc1)c1ccc(OCC(O)COC2CCCCC2)cc1] | 1-chloro-3-(4-(2-(4-(3-(cyclohexyloxy)- 2-hydroxypropoxy)phenyl)propan-2- yl)phenoxy)propan-2-ol |
| 49 | 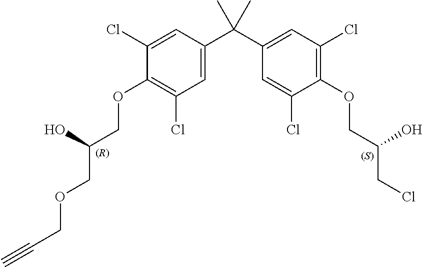 | (S)-1-chloro-3-(2,6-dichloro-4- (2-(3,5-dichloro-4-((R)-2-hydroxy-3- (prop-2-ynyloxy)propoxy)phenyl) propan-2-yl)phenoxy)propan-2-ol |
| 50 | 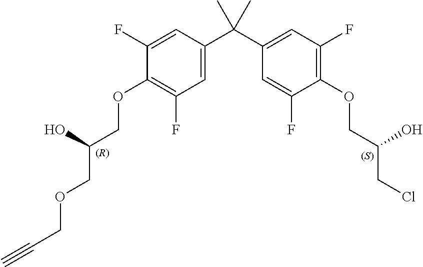 | (S)-1-chloro-3-(4-(2-(3,5-difluoro-4- ((R)-2-hydroxy-3-(prop-2- ynyloxy)propoxy)phenyl)propan-2-yl)- 2,6-difluorophenoxy)propan-2-ol |
| 51 | 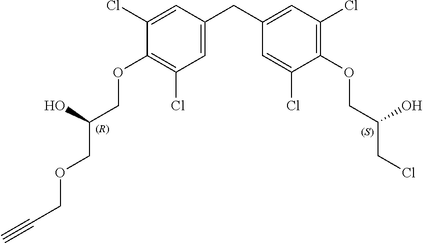 | (S)-1-chloro-3-(2,6-dichloro-4-(3,5- dichloro-4- ((R)-2-hydroxy-3-(prop-2- ynyloxy)propoxy)benzyl)phenoxy) propan-2-ol |
| 52 | 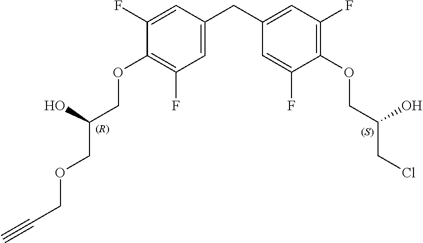 | (S)-1-chloro-3-(4-(3,5-difluoro-4- ((R)-2-hydroxy-3-(prop-2-ynyloxy) propoxy)benzyl)-2,6- difluorophenoxy)propan-2-ol |
| 53 | 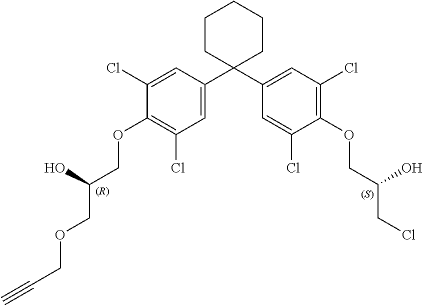 | (S)-1-chloro-3-(2,6-dichloro-4- (1-(3,5-dichloro-4-((R)-2-hydroxy-3- (prop-2-ynyloxy)propoxy)phenyl) cyclohexyl) phenoxy)propan-2-ol |
| 54 | 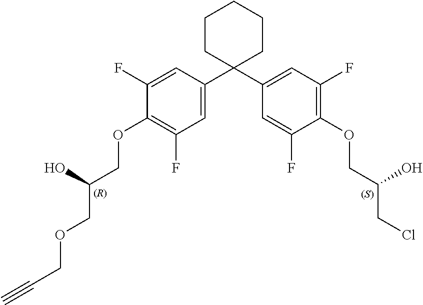 | (S)-1-chloro-3-(4-(1-(3,5-difluoro-4- ((R)-2-hydroxy-3-(prop-2-ynyloxy) propoxy)phenyl)cyclohexyl)-2,6- difluorophenoxy)propan-2-ol |

Prodrugs are also included within the scope of the present disclosure. For example, in one embodiment the hydrogen atom of one or more hydroxyl groups of any of the compounds of Formula I may be replaced with a moiety from Table 1. Non-limiting examples of such prodrugs include glycine esters and salts thereof as shown below.

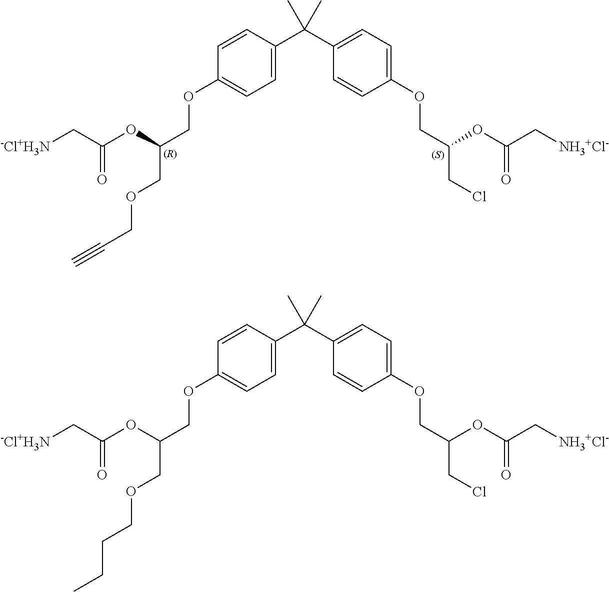

In some embodiments, the compounds as described herein or acceptable salts, tautomers or stereoisomers thereof above may be used for systemic treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. In some embodiments, the compounds as described herein or acceptable salts thereof above may be used in the preparation of a medicament or a composition for systemic treatment of an indication described herein. In some embodiments, methods of systemically treating any of the indications described herein are also provided. Some aspects of this invention, make use of compositions comprising a compound described herein and a pharmaceutically acceptable excipients or carrier. In some embodiments, the prostate cancer is castration-resistant prostate cancer (also referred to as hormone refractory, androgen-independent, androgen deprivation resistant, androgen ablation resistant, androgen depletion-independent, castration-recurrent, anti-androgen-recurrent). In some embodiments the prostate cancer is androgen-dependent or androgen-sensitive. Methods of treating any of the indications described herein are also provided. Such methods may include administering a compound as described herein or a composition of a compound as described herein, or an effective amount of a compound as described herein or composition of a compound as described herein to a subject in need thereof.

Compounds as described herein may be in the free form or in the form of a salt thereof. In some embodiments, compounds as described herein may be in the form of a pharmaceutically acceptable salt, which are known in the art (Berge et al., J. Pharm. Sci. 1977, 66, 1). Pharmaceutically acceptable salt as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid. Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylamino ethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, 1-ephenamine, N,N′-dibenzylethylenediamine or polyamine resins. In some embodiments, compounds as described herein may contain both acidic and basic groups and may be in the form of inner salts or zwitterions, for example, and without limitation, betaines. Salts as described herein may be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

In some embodiments, compounds and all different forms thereof (e.g. free forms, salts, polymorphs, isomeric forms) as described herein may be in the solvent addition form, for example, solvates. Solvates contain either stoichiometric or non-stoichiometric amounts of a solvent in physical association the compound or salt thereof. The solvent may be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formula illustrated for the sake of convenience.

In some embodiments, pharmaceutical compositions in accordance with this invention may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to an androgen-independent form. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In some embodiments, compounds and all different forms thereof as described herein may be used, for example, and without limitation, in combination with other treatment methods for at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. For example, compounds and all their different forms as described herein may be used as neoadjuvant (prior), adjunctive (during), and/or adjuvant (after) therapy with surgery, radiation (brachytherapy or external beam), or other therapies (eg. HIFU), and in combination with chemotherapies, androgen ablation, antiandrogens or any other therapeutic approach.

With respect to combination therapies, one embodiment of the present disclosure provides a combination of any one or more of a compound of Formula I with one or more currently-used or experimental pharmacological therapies which are or may be utilized to treat any of the above disease states (e.g., androgen-independent prostate cancer or Kennedy's disease). Methods, uses and pharmaceutical compositions comprising the above combination are also provided. Combination therapies for such indications are disclosed in co-pending U.S. Provisional Application No. 61/384,628, which is hereby incorporated by reference in its entirety.

Surprisingly, it has been found that the disclosed compounds, which interfere with the AR principally through binding to the N-terminus of the AR, demonstrate beneficial synergistic therapeutic effects when used in concert with existing approved and in-development agents. That is, the biological impact of using the agents in concert with one another produces a biological and therapeutic effect which is greater than the simple additive effect of each of them separately.

Accordingly, one embodiment comprises the use of the disclosed compounds in combination therapy with one or more currently-used or experimental pharmacological therapies which are utilized for treating the above disease states irrespective of the biological mechanism of action of such pharmacological therapies, including without limitation pharmacological therapies which directly or indirectly inhibit the androgen receptor, pharmacological therapies which are cyto-toxic in nature, and pharmacological therapies which interfere with the biological production or function of androgen (hereinafter, the “Other Therapeutic Agents”). By “combination therapy” is meant the administration of any one or more of a coumpound of Formula I with one or more of another therapueitc agent to the same patient such that their pharmacological effects are contemporaneous with one another, or if not contemporaneous, that their effects are synergistic with one another even though dosed sequentially rather than contemporaneously.

Such administration includes without limitation dosing of one or more of a compound of Formula I and one or more of the Other Therapeutic Agent(s) as separate agents without any comingling prior to dosing, as well as formulations which include one or more Other Androgen-Blocking Therapeutic Agents mixed with one or more compound of Formula I as a pre-mixed formulation. Administration of the compound(s) of Formula I in combination with Other Therapeutic Agents for treatment of the above disease states also includes dosing by any dosing method including without limitation, intravenous delivery, oral delivery, intra-peritoneal delivery, intra-muscular delivery, or intra-tumoral delivery.

In another aspect of the present disclosure, the one or more of the Other Therapeutic Agent may be administered to the patient before administration of the compound(s) of Formula I. In another embodiment, the compound(s) of Formula I may be co-administered with one or more of the Other Therapeutic Agents. In yet another aspect, the one or more Other Therapeutic Agent may be administered to the patient after administration of the compound(s) of Formula I.

It is fully within the scope of the disclosure that the ratio of the doses of compound(s) of Formula I to that of the one or more Other Therapeutic Agents may or may not equal to one and may be varied accordingly to achieve the optimal therapeutic benefit.

For greater clarity the compound(s) of Formula I that are combined with the one or more Other Therapeutic Agents for improved treatment of the above disease states may comprise, but are not limited to any compound having a structure of Formula I, including those compounds shown in Table 2.

The Other Therapeutic Agents include without limitation any pharmacological agent which is currently approved by the FDA in the U.S. (or elsewhere by any other regulatory body) for use as pharmacological treatment of any of the above disease states, or which is currently being used experimentally as part of a clinical trial program that relates to the above disease states. Non-limiting examples of the Other Pharmacological Agents comprise, without limitation: the chemical entity known as MDV3100 (4-(3-(4-cyano-3-(trifluoromethyl)phenyl)-5,5-dimethyl-4-oxo-2-thioxoimidazolidin-1-yl)-2-fluoro-N-methylbenzamide) and related compounds, which appears to be a blocker of the AR LBD and is currently in development as a treatment for prostate cancer; the chemical entity known as TOK 001 and related compounds which appears to be a blocker of the AR LBD, and a CYP17 lyase inhibitor, and also appears to decrease overall androgen receptor levels in prostate cancer cells. TOK 001 is currently in development as a treatment for prostate cancer; the chemical entity known as ARN-509 and related compounds which appears to be a blocker of the AR LBD and is currently in development as a treatment for prostate cancer; the chemical entity known as abiraterone (or CB-7630; (3S,8R,9S,10R,13S,14 S)-10,13-dimethyl-17-(pyridin-3-yl) 2,3,4,7,8,9,10,11,12,13,14,15-dodecahydro-1H-cyclopenta[a]phenanthren-3-ol), and related molecules, which appears to block the production of androgen and is currently in development for the treatment of prostate cancer; the chemical entity known as bicalutamide (N-[4-cyano-3-(trifluoromethyl)phenyl]-3-[(4-fluorophenyl)sulfonyl]-2-hydroxy-2-methylpropanamide) and related compounds, which appears to be a blocker of the AR LBD and which is currently used to treat prostate cancer, the chemical entity known as nilutamide (5,5-dimethyl-3-[4-nitro-3-(trifluoromethyl)phenyl]imidazolidine-2,4-dione) and related compounds, which appears to be a blocker of the AR LBD and which is currently used to treat prostate cancer, the chemical entity known as flutamide (2-methyl-N-[4-nitro-3-(trifluoromethyl)phenyl]-propanamide) and related compounds, which appears to be a blocker of the AR LBD and which is currently used to treat prostate cancer, the chemical entities know as cyproterone acetate (6-chloro-1β,2β-dihydro-17-hydroxy-3′H-cyclopropa[1,2]pregna-4,6-diene-3,20-dione) and related compounds, which appears to be a blocker of the AR LBD and which is currently used to treat prostate cancer, the chemical entity known as docetaxel (Taxotere; 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}) and related compounds, which appears to be a cytotoxic antimicrotubule agent and is currently used in combination with prednisone to treat prostate cancer, the chemical entity known as Bevacizumab (Avastin), a monoclonal antibody that recognizes and blocks vascular endothelial growth factor A (VEGF-A) and may be used to treat prostate cancer, the chemical entity known as OSU-HDAC42 ((S)-(+)-N-hydroxy-4-(3-methyl-2-phenylbutyrylamino)-benzamide), and related compounds, which appears to act as a histone deacetylase inhibitor, and is currently being developed as a treatment for prostate cancer, the chemical entity known as VITAXIN which appears to be a monoclonal antibody against the vascular integrin ανβ3 to prevent angiogenesis, and which may be used to treat prostate cancer, the chemical entity known as sunitumib (N-(2-diethylaminoethyl)-5-[(Z)-(5-fluoro-2-oxo-1H-indol-3-ylidene)methyl]-2,4-dimethyl-1H-pyrrole-3-carboxamide) and related compounds, which appears to inhibit multiple receptor tyrosine kinases (RTKs) and may be used for treatment of prostate cancer, the chemical entity known as ZD-4054 (N-(3-Methoxy-5-methylpyrazin-2-yl)-2-[4-(1,3,4-oxadiazol-2-yl)phenyl]pyridin-3-sulfonamid) and related compounds, which appears to block the edta receptor and which may be used for treatment of prostate cancer, the chemical entity known as VN/124-1 (3β-Hydroxy-17-(1H, benzimidazol-1-yl)androsta-5,16-diene), and related compounds which appears to block the production of androgen (via inhibition of-hydroxylase/17,20 lyase) and is currently in development for the treatment of prostate cancer; the chemical entity known as Cabazitaxel (XRP-6258), and related compounds, which appears to be a cytotoxic microtubule inhibitor, and which is currently used to treat prostate cancer; the chemical entity known as MDX-010 (Ipilimumab), a fully human monoclonal antibody that binds to and blocks the activity of CTLA-4 which is currently in development as an immunotherapeutic agent for treatment of prostate cancer; the chemical entity known as OGX 427 which appears to target HSP27 as an antisense agent, and which is currently in development for treatment of prostate cancer; the chemical entity known as OGX 011 which appears to target clusterin as an antisense agent, and which is currently in development as a treatment for prostate cancer; the chemical entity known as finasteride (Proscar, Propecia; N-(1,1-dimethylethyl)-3-oxo-(5α, 17β)-4-azaandrost-1-ene-17-carboxamide), and related compounds, which appears to be a 5-alpha reductase inhibitor that reduces levels of dihydrotestosterone, and may be used to treat prostate cancer; the chemical entity known as dutasteride (Avodart; 5α, 17β)-N-{2,5 bis(trifluoromethyl)phenyl}-3-oxo-4-azaandrost-1-ene-17-carboxamide) and related molecules, which appears to be a 5-alpha reductase inhibitor that reduces levels of dihydrotestosterone, and may be used in the treatment of prostate cancer; the chemical entity known as turosteride ((4aR,4bS,6aS,7S,9aS,9bS,11aR)-1,4-a,6a-trimethyl-2-oxo-N-(propan-2-yl)-N-(propan-2-ylcarbamoyl)hexadecahydro-1H-indeno[5,4-f]quinoline-7-carboxamide), and related molecules, which appears to be a 5-alpha reductase inhibitor that reduces levels of dihydrotestosterone and may be used in the treatment of prostate cancer; the chemical entity known as bexlosteride (LY-191,704; (4a S,10bR)-8-chloro-4-methyl-1,2,4a,5,6,10b-hexahydrobenzo[f]quinolin-3-one), and related compounds, which appears to be a 5-alpha reductase inhibitor that reduces levels of dihydrotestosterone and may be used in the treatment of prostate cancer; the chemical entity known as izonsteride (LY-320,236; (4 aR,10bR)-8-[(4-ethyl-1,3-benzothiazol-2-yl)sulfanyl]-4,10b-dimethyl-1,4,4a,5,6,10b-hexahydrobenzo[f]quinolin-3(2H)-one) and related compounds, which appears to be a 5-alpha reductase inhibitor that reduces levels of dihydrotestosterone and may be used for the treatment of prostate cancer; the chemical entity known as FCE 28260 and related compounds, which appears to be a 5-alpha reductase inhibitor that reduces levels of dihydrotestosterone and may be used for the treatment of prostate cancer; the chemical entity known as SKF105,111, and related compounds, which appears to be a 5-alpha reductase inhibitor that reduces levels of dihydrotestosterone and may be used for treatment of prostate cancer.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions. Some compounds of this invention may be toxic at some concentrations. Titration studies may be used to determine toxic and non-toxic concentrations. Toxicity may be evaluated by examining a particular compound's or composition's specificity across cell lines using PC3 cells as a negative control that do not express functional AR. Animal studies may be used to provide an indication if the compound has any effects on other tissues. Systemic therapy that targets the AR will not likely cause major problems to other tissues since antiandrogens and androgen insensitivity syndrome are not fatal.

Compounds as described herein may be administered to a subject. As used herein, a “subject” may be a human, non-human primate, mammal, rat, mouse, cow, horse, pig, sheep, goat, dog, cat and the like. The subject may be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

Compounds described herein may be used for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. Compounds described herein may be used for treatment of prostate cancer. Compounds described herein may be used for treatment of castration-resistant prostate cancer. Compounds described herein may be used for treatment of androgen-dependent prostate cancer. Compounds described herein may be used for preparation of a medicament for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. Compounds described herein may be used for the preparation of a medicament for treatment of prostate cancer. Compounds described herein may be used for the preparation of a medicament for treatment of castration-resistant prostate cancer. Compounds described herein may be used for the preparation of a medicament for treatment of androgen-dependent prostate cancer. Compounds described herein may be used in a method for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration. The method may comprise administering to a subject in need thereof an effective amount of a compound described herein. Compounds described herein may be used in a method of treatment of prostate cancer, the method comprising administering to a subject in need thereof an effective amount of a compound described herein. Compounds described herein may be used in a method of treatment of castration resistant prostate cancer, the method comprising administering to a subject in need thereof an effective amount of a compound described herein. Compounds described herein may be used in a method of treatment of androgen-dependent prostate cancer, the method comprising administering to a subject in need thereof an effective amount of a compound described herein.

Compounds described herein may also be used in assays and for research purposes. Definitions used include ligand-dependent activation of the androgen receptor (AR) by androgens such as dihydrotestosterone (DHT) or the synthetic androgen (R1881) used for research purposes. Ligand-independent activation of the AR refers to transactivation of the AR in the absence of androgen (ligand) by, for example, stimulation of the cAMP-dependent protein kinase (PKA) pathway with forskolin (FSK). Some compounds and compositions of this invention may inhibit both FSK and androgen (e.g. R1881) induction of ARE-luciferase (ARE-luc). Constitutive activity of the AR refers to splice variants lacking the AR ligand-binding domain. Such compounds may block a mechanism that is common to both ligand-dependent and ligand-independent activation of the AR, as well as constitutively active splice variants of the AR that lack ligand-binding domain. This could involve any step in activation of the AR including dissociation of heatshock proteins, essential posttranslational modifications (e.g., acetylation, phosphorylation), nuclear translocation, protein-protein interactions, formation of the transcriptional complex, release of co-repressors, and/or increased degradation. Some compounds and compositions of this invention may inhibit ligand-only activity and may interfere with a mechanism specific to ligand-dependent activation (e.g., accessibility of the ligand binding domain (LBD) to androgen). Numerous disorders in addition to prostate cancer involve the androgen axis (e.g., acne, hirsutism, alopecia, benign prostatic hyperplasia) and compounds interfering with this mechanism may be used to treat such conditions. Some compounds and compositions of this invention may only inhibit FSK induction and may be specific inhibitors to ligand-independent activation of the AR. These compounds and compositions may interfere with the cascade of events that normally occur with FSK and/or PKA activity or any downstream effects that may play a role on the AR (e.g. FSK increases MAPK activity which has a potent effect on AR activity). Examples may include an inhibitor of cAMP and or PKA or other kinases. Some compounds and compositions of this invention may induce basal levels of activity of the AR (no androgen or stimulation of the PKA pathway). Some compounds and compositions of this invention may increase induction by R1881 or FSK. Such compounds and compositions may stimulate transcription or transactivation of the AR. Some compounds and compositions of this invention may inhibit activity of the androgen receptor. Interleukin-6 (IL-6) also causes ligand-independent activation of the AR in LNCaP cells and can be used in addition to FSK.

Compounds for use in the present invention may be obtained from medical sources or modified using known methodologies from naturally occurring compounds. In addition, methods of preparing or synthesizing compounds of the present invention will be understood by a person of skill in the art having reference to known chemical synthesis principles. For example, Auzou et al 1974 European Journal of Medicinal Chemistry 9(5), 548-554 describes suitable synthetic procedures that may be considered and suitably adapted for preparing compounds of any one of the Formula I to XXI as set out above. Other references that may be helpful include: Debasish Das, Jyh-Fu Lee and Soofin Cheng “Sulfonic acid functionalized mesoporous MCM-41 silica as a convenient catalyst for Bisphenol-A synthesis” Chemical Communications, (2001) 2178-2179; U.S. Pat. No. 2,571,217 Davis, Orris L.; Knight, Horace S.; Skinner, John R. (Shell Development Co.) “Halohydrin ethers of phenols.” (1951); and Rokicki, G.; Pawlicki, J.; Kuran, W. “Reactions of 4-chloromethyl-1,3-dioxolan-2-one with phenols as a new route to polyols and cyclic carbonates.” Journal fuer Praktische Chemie (Leipzig) (1985) 327, 718-722.

For example, compounds of the present invention wherein n is 1, J is O and each of L and L² is OH may be obtained with reference to the following general Synthetic Scheme 1:

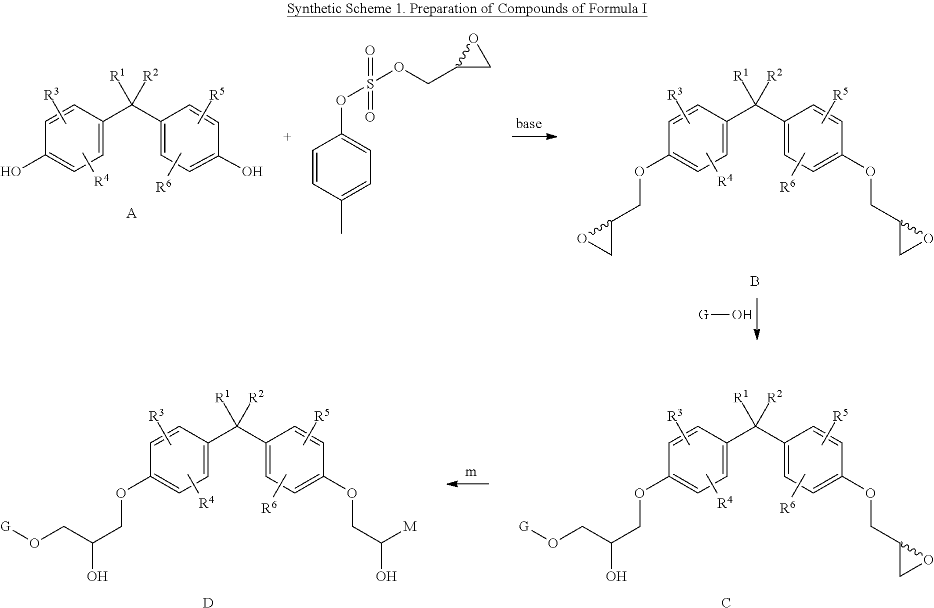

Referring to Synthetic Scheme 1, compounds of structure A can be purchased from commercial sources or prepared according to methods known in the art. Epoxidation of A with an appropriate reagent, for example glycidyl tosylate, results in compounds of structure B. Various epoxidation reagents may be employed, including optically pure reagents which yield optically pure epoxides (e.g., + or − glycidyl tosylate). Treatment of B with an appropriately substituted alcohol yields C. In certain embodiments, use of a catalyst (e.g., Er(OTf)₃) may aid the epoxide ring opening reaction. Finally, reaction of C with an appropriate nucleophile (“m”), for example Cl⁻ or OH⁻ and the like, results in compounds of structure D.

One skilled in the art will recognize that variations to the order of the steps and reagents discussed in reference to Synthetic Scheme I are possible. For example, a single hydroxyl moiety may be epoxidized followed by epoxide ring opening. In an analogous manner, the second hydroxyl can then be epoxidized and the epoxide opened. The order of functionalizing the various epoxides may also be varied (e.g., add “m” before G-OH). Finally, further compounds of Formual I can be prepared by functionalizing (or removing) one or both of the free hydroxyl groups (i.e., L and L²) of D. Methods for such functionalization are well-known in the art, for example reaction with an acid chloride analogue of a moiety from TABLE 1 or any other suitable reagent. Methodologies for preparation of compounds of Formula I are described in more detail in the following non-limiting exemplary schemes.

Various alternative embodiments and examples of the invention are described herein. These embodiments and examples are illustrative and should not be construed as limiting the scope of the invention.

### EXAMPLES

All non-aqueous reactions were performed in flame-dried round bottomed flasks. The flaks were fitted with rubber septa and reactions were conducted under a positive pressure of argon unless otherwise specified. Stainless steel syringes were used to transfer air- and moisture-sensitive liquids. Flash column chromatography was performed as described by Still et al. (Still, W. C.; Kahn, M.; Mitra, A. J. Org. Chem. 1978, 43, 2923) using 230-400 mesh silica gel. Thin-layer chromatography was performed using aluminium plates pre-coated with 0.25 mm 230-400 mesh silica gel impregnated with a fluorescent indicator (254 nm). Thin-layer chromatography plates were visualized by exposure to ultraviolet light and a “Seebach” staining solution (700 mL water, 10.5 g Cerium (IV) sulphate tetrahydrate, 15.0 g molybdato phosphoric acid, 17.5 g sulphuric acid) followed by heating (˜1 min) with a heating gun (˜250° C.). Organic solutions were concentrated on Büchi R-114 rotatory evaporators at reduced pressure (15-30 ton, house vacuum) at 25-40° C.

Commercial regents and solvents were used as received. All solvents used for extraction and chromatography were HPLC grade. Normal-phase Si gel Sep Paks™ were purchased from waters, Inc. Thin-layer chromatography plates were Kieselgel 60F₂₅₄. All synthetic reagents were purchased from Sigma Aldrich and Fisher Scientific Canada.

Proton nuclear magnetic resonance (¹H NMR) spectra were recorded at 25° C. using a Bruker 400 with inverse probe and Bruker 400 spectrometers, are reported in parts per million on the δ scale, and are referenced from the residual protium in the NMR solvent (DMSO-d₆: δ 2.50 (DMSO-d₅), CDCl₃: δ 7.24 (CHCl₃)). Carbon-13 nuclear magnetic resonance (¹³C NMR) spectra were recorded with a Bruker 400 spectrometer, are reported in parts per million on the δ scale, and are referenced from the carbon resonances of the solvent (DMSO-d₆: δ 39.51, CDCl₃: δ 77.00). Spectral features are tabulated in the following order: chemical shift (δ, ppm); multiplicity (s=singlet, d=doublet, t=triplet, m=multiplet, br=broad); coupling constant (J, Hz, number of protons).

LNCaP cells were employed initially for all experiments because they are well-differentiated human prostate cancer cells in which ligand-independent activation of the AR by FSK has been characterized (Nazareth et al 1996 J. Biol. Chem. 271, 19900-19907; and Sadar 1999 J. Biol. Chem. 274, 7777-7783). LNCaP cells express endogenous AR and secrete prostate-specific antigen (PSA) (Horoszewicz et al 1983Cancer Res. 43, 1809-1818). LNCaP cells can be grown either as monolayers in cell culture or as tumors in the well-characterized xenograft model that progresses to androgen independence in castrated hosts (Sato et al 1996 J. Steroid Biochem. Mol. Biol. 58, 139-146; Gleave et al 1991 Cancer Res. 51, 3753-3761; Sato et al 1997 Cancer Res. 57, 1584-1589; and Sadar et al 2002 Mol. Cancer. Ther. 1(8), 629-637). R1881 was employed since it is stable and avoids problems associated with the labile physiological ligand dihydrotestosterone (DHT). Reporter specificity may be determined using several alternative reporter gene constructs. Some well characterized ARE-driven reporter gene constructs that have been used extensively are the PSA (6.1 kb) enhance/promoter which contains several AREs and is highly inducible by androgens as well as by FSK (Ueda et al 2002 A J. Biol. Chem. 277, 7076-7085) and the ARR3-thymidine kinase (tk)-luciferase, which is an artificial reporter construct that contains three tandem repeats of the rat probasin ARE1 and ARE2 regions upstream of a luciferase reporter (Snoek et al 1996 J. Steroid Biochem. Mol. Biol. 59, 243-250).

### Example 1

### Synthesis of 1-chloro-3-(4-(2-(4-(oxiran-2-ylmethoxy)phenyl)propan-2-yl)phenoxy)propan-2-ol (2)

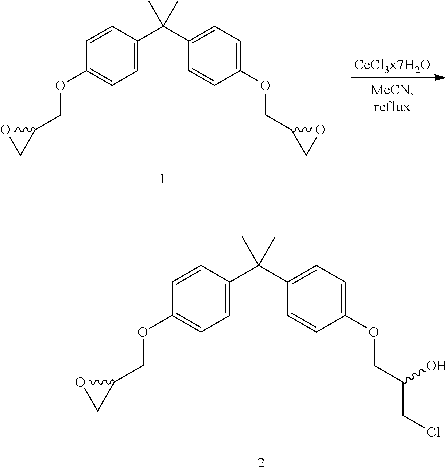 [SMILES: CC(C)(c1ccc(OCC2CO2)cc1)c1ccc(OCC2CO2)cc1 ; CC(C)(c1ccc(OCC(O)CCl)cc1)c1ccc(OCC2CO2)cc1]

To a solution of racemic derivative Bisphenol A diglycidyl ether 1 (13.30 g, 39.27 mmol, 1 equiv) in acetonitrile (30 mL) was added CeCl₃.7H₂O (7.30 g, 19.63 mmol, ½ equiv) and the mixture was refluxed for 3.5 h. The resulting white paste was filtered and washed with ethyl acetate, and the clear suspension was concentrated under reduced pressure. The resulting residue was purified by flash column chromatography on silica gel (eluent: 10% ethyl acetate in hexane) to provide 2 (2.12 g, 14%) as a pale liquid.

¹H NMR (400 MHz, DMSO-d₆): δ 7.10 (d, J=8.4, 4H), 6.86-6.81 (dd, J=9.2, 3.6, 4H), 5.50 (d, J=5.2, 1H), 4.28-4.25 (dd, J=11.2, 2.4, 1H), 4.04-3.99 (m, 1H), 3.93 (d, J=5.2, 2H), 3.81-3.77 (dd, J=11.6, 6.4, 1H), 3.76-3.72 (dd, J=11.2, 4.4, 1H), 3.67-3.63 (dd, J=10.8, 5.2, 1H), 3.31-3.29 (m, 1H), 2.83 (t, J=4.4, 1H), 2.70-2.68 (dd, J=5.2, 2.8, 1H), 1.58 (s, 6H); ¹³C NMR (100 MHz, DMSO-d₆): δ 156.1, 156.0, 142.9, 142.8, 127.4, 113.9, 68.8, 68.6, 49.7, 46.7, 43.7, 41.2, 30.7; HRMS (ESI) (m/z): calc'd for C₂₁H₂₅O₄NaCl [M+Na]⁺:399.1339. found: 399.1348.

### Example 2

### Synthesis of 1-chloro-3-(4-(2-(4-(2-hydroxy-3-(prop-2-ynyloxy)propoxy)phenyl)propan-2-YL)phenoxy)propan-2-ol (3)

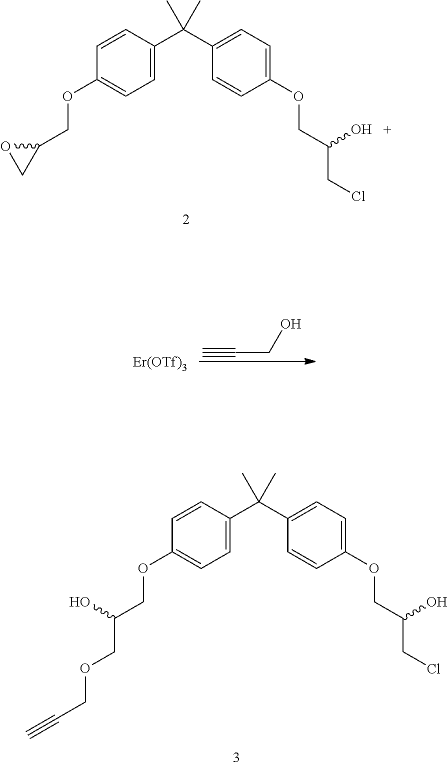

Propargyl alcohol (1.5 mL) was added to racemic derivative 2 (111 mg, 0.29 mmol, 1 equiv) and the mixture was stirred for 10 min. Solid Erbium(III) trifluoromethanesulfonate (36 mg, 0.058 mmol, 1/5 equiv) was added in one portion and the solution was stirred at room temperature for 19 h. Sodium bicarbonate was added (1 mL) and the reaction was extracted with dichloromethane (3×5 mL). The organic layer was washed with deionized water (5 mL), dried over anhydrous magnesium sulfate, filtered and concentrated under reduced pressure. The resulting residue was purified by flash column chromatography on silica gel Sep pak (5 g) (eluent: 30% ethyl acetate in hexane) to provide 3 (87 mg, 68%) as a transparent foam. ¹H NMR (400 MHz, DMSO-d₆): δ 7.11-7.08 (dd, J=8.8, 3.6, 4H), 6.85-6.81 (dd, J=8.4, 6.8, 4H), 5.52 (d, J=5.6, 1H), 5.13 (d, J=4.4, 1H), 4.16 (d, J=2.4, 2H), 4.03-3.99 (m, 1H), 3.95-3.88 (m, 4H), 3.86-3.82 (m, 1H), 3.76-3.72 (dd, J=11.2, 4.4, 1H), 3.67-3.63 (dd, J=10.8, 5.2, 1H), 3.55-3.46 (m, 2H), 3.43 (t, J=2.0, 1H), 1.57 (s, 6H); ¹³C NMR (100 MHz, DMSO-d₆): δ 156.3, 156.1, 142.9, 142.7, 127.4, 127.4, 113.9, 80.3, 77.2, 71.0, 69.4, 68.8, 68.6, 67.8, 57.9, 46.8, 41.2, 30.7; HRMS (ESI) (m/z): calc'd for C₂₄H₂₉O₅NaCl [M+Na]⁺:455.1601. found: 455.1602.

### Example 3

### Synthesis of 3-(4-(2-(4-(2-hydroxy-3-(prop-2-ynyloxy)propoxy)phenyl)propan-2-yl)phenoxy)propane-1,2-diol (5)

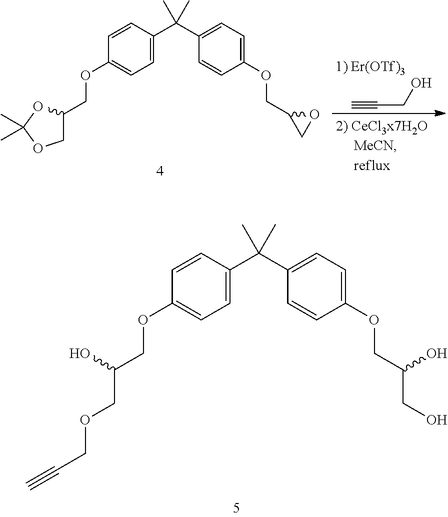

Propargyl alcohol (5 mL) was added to racemic derivative 4 (377 mg, 0.94 mmol, 1 equiv), and the mixture was stirred for 10 min. Solid Erbium(III) trifluoromethanesulfonate (116 mg, 0.19 mmol, 1/5 equiv) was added in one portion, and the solution was stirred at room temperature for 24 h. Sodium bicarbonate was added (2 mL), and the reaction was extracted with dichloromethane (3×8 mL). The organic layer was washed with deionized water (10 mL), dried over anhydrous magnesium sulfate, filtered and concentrated to dryness under reduced pressure. The crude residue was dissolved in acetonitrile (2 mL), and solid CeCl₃.7H₂O (100 mg, 0.27 mmol) was added in one portion. The mixture was refluxed for 4 h. The resulting white paste was filtered and washed with ethyl acetate, and the clear suspension was concentrated under reduced pressure. The resulting residue was purified by flash column chromatography on silica gel Sep pak (5 g) (eluent: 5% methanol in dichloromethane) to provide 5 (81 mg, 21%, 2 steps) as a foam. ¹H NMR (400 MHz, DMSO-d₆): δ 7.09 (d, J=8.8, 4H), 6.81 (d, J=8.8, 4H), 5.13 (d, J=5.2, 1H), 4.89 (d, J=4.8, 1H), 4.62 (t, J=5.6, 1H), 4.16 (d, J=2.4, 2H), 3.96-3.88 (m, 3H), 3.86-3.74 (m, 3H), 3.55-3.46 (m, 2H), 3.44-3.42 (m, 3H), 1.57 (s, 6H);

¹³C NMR (100 MHz, DMSO-d₆): δ 156.5, 156.3, 142.7, 142.5, 127.4, 127.4, 113.8, 113.8, 80.3, 77.2, 71.0, 69.9, 69.4, 69.4, 67.8, 62.7, 57.9, 41.1, 30.7; HRMS (ESI) (m/z): calc'd for C₂₄H₃₀O₆Na [M+Na]⁺: 437.1940. found: 437.1929.

### Example 4

### Synthesis of (S)-4-(2-(4-(oxiran-2-ylmethoxy)phenyl)propan-2-yl)phenol (7)

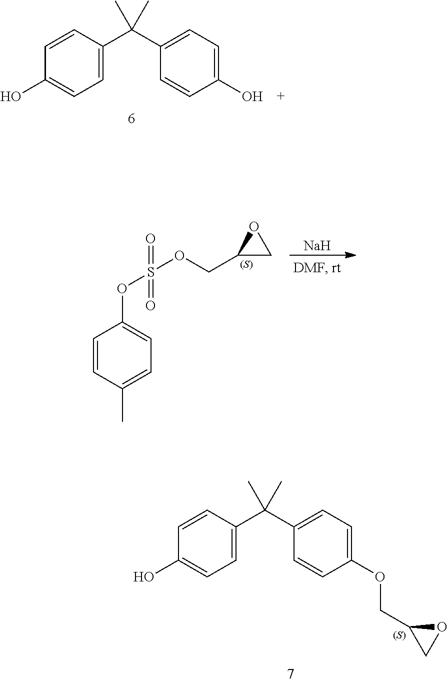

Sodium hydride (60% dispersion in mineral oil, 175 mg, 4.38 mmol, 1.0 equiv) was added slowly to a stirred solution of Bisphenol A 6 (1000 mg, 4.38 mmol, 1 equiv) in anhydrous dimethyl formamide (4 mL) at room temperature, and the contents were stirred under an atmosphere of argon for 20 min. A solution of (2S)-(+)-glycidyl tosylate 98% (1200 mg, 5.25 mmol, 1.2 equiv) in anhydrous dimethyl formamide (4 mL) was added slowly via syringe, and the mixture was allowed to react at room temperature for 15 h. Then, the reaction was quenched by the addition of a saturated solution of ammonium chloride (5 mL), and the mixture was extracted with ethyl acetate (3×15 mL). The organic layer was washed with deionized water (15 mL), dried over anhydrous magnesium sulfate, filtered and concentrated under reduced pressure. The resulting residue was purified by flash column chromatography on silica gel (eluent: 10% ethyl acetate in hexane) to provide 7 (372 mg, 36%) as a clear foam. Note: The same reaction also yielded the bis epoxide together with unreacted starting material.

### Example 5

### Synthesis of (S)-4-(2-(4-(2-hydroxy-3-(prop-2-ynyloxy)propoxy)phenylpropan-2-yl)phenol (8)

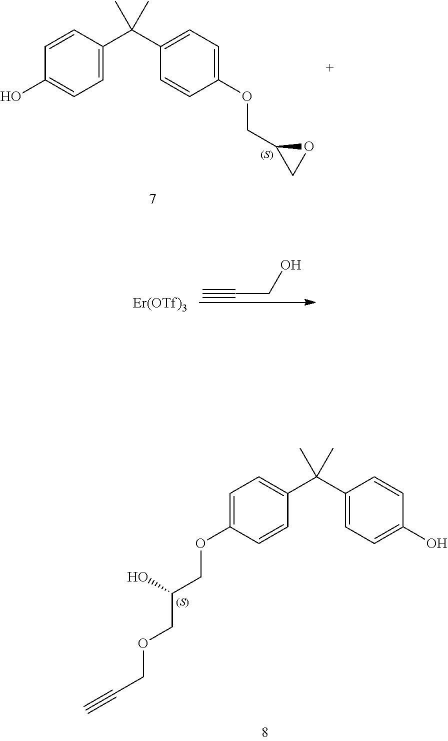

Propargyl alcohol (3.0 mL) was added to derivative 7 (372 mg, 1.31 mmol, equiv), and the mixture was stirred for 10 min. Solid Erbium(III) trifluoromethanesulfonate (161 mg, 0.26 mmol, 1/5 equiv) was added in one portion and the solution was stirred at room temperature for 12 h. Sodium bicarbonate was added (1 mL), and the reaction was extracted with dichloromethane (3×5 mL). The organic layer was washed with deionized water (5 mL), dried over anhydrous magnesium sulfate, filtered and concentrated under reduced pressure. The resulting residue was purified by flash column chromatography on silica gel (eluent: 20% ethyl acetate in hexane to 40% ethyl acetate) to provide 8 (355 mg, 78%) as a pale foam. ¹H NMR (400 MHz, DMSO-d₆): δ 9.14 (S, 1H), 7.08 (d, J=8.8, 2H), 6.98 (d, J=8.8, 2H), 6.81 (d, J=8.8, 2H), 6.65 (d, J=8.4, 2H), 5.14 (d, J=4.8, 1H), 4.16 (d, J=2.4, 2H), 3.94-3.89 (m, 2H), 3.86-3.84 (m, 1H), 3.55-3.47 (m, 2H), 3.41 (t, J=2.0, 1H), 1.55 (s, 6H); ¹³C NMR (100 MHz, DMSO-d₆): δ 156.9, 155.6, 143.5, 141.4, 128.0, 127.9, 115.2, 114.4, 80.9, 77.7, 71.6, 70.0, 68.5, 58.5, 41.6, 31.4; HRMS (ESI) (m/z):calc'd for C₂₁H₂₄O₄Na [M+Na]⁺: 363.1572. found: 363.1577.

### Example 6

### Synthesis of (S)-1-(4-(2-(4-((S)-oxiran-2-ylmethoxyphenyl)propan-2-yl)phenoxy)-3-(prop-2-ynyloxy)propan-2-ol (9)

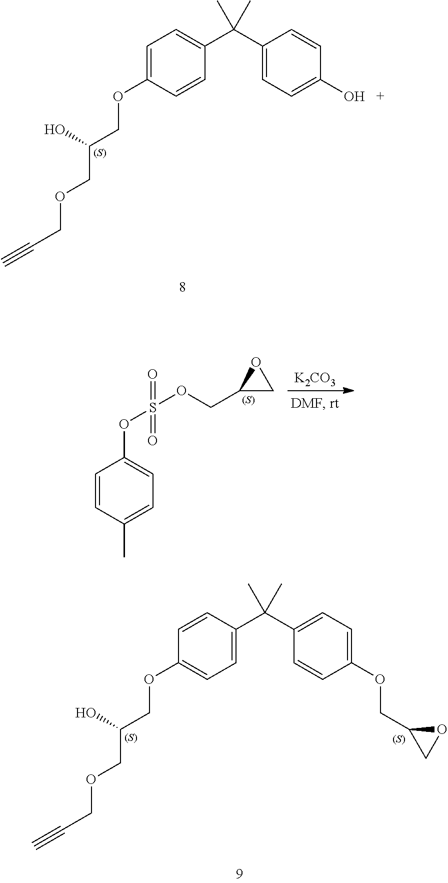

Potassium carbonate anhydrous (113 mg, 0.82 mmol, 2.0 equiv) was added to a stirred solution of derivative 8 (140 mg, 0.41 mmol, 1 equiv) in anhydrous dimethyl formamide (2 mL) at room temperature, and the contents were stirred under an atmosphere of argon for 20 min. A solution of (25)-(+)-glycidyl tosylate 98% (187 mg, 0.82 mmol, 2.0 equiv) in anhydrous dimethyl formamide (1 mL) was added slowly via syringe, and the mixture was allowed to react at room temperature for 84 h. Then, the reaction was quenched by the addition of a saturated solution of ammonium chloride (1 mL), and the mixture was extracted with ethyl acetate (3×5 mL). The organic layer was washed with deionized water (5 mL), dried over anhydrous magnesium sulfate filtered and concentrated under reduced pressure. The resulting residue was purified by flash column chromatography on silica gel (eluent: 10% ethyl acetate in hexane to 40% ethyl acetate) to provide 9 (6S,24S) (154 mg, 94%) as a clear foam. ¹H NMR (400 MHz, DMSO-d₆): δ 7.11-7.08 (m, 4H), 6.85-6.81 (m, 4H), 5.14 (d, J=5.2, 1H), 4.29-4.27 (dd, J=4.8, 2.4, 1H), 4.16 (d, J=2.0, 2H), 3.94-3.88 (m, 2H), 3.85-3.76 (m, 2H), 3.55-3.48 (m, 2H), 3.43 (t, J=2.4, 1H), 3.31-3.29 (m, 1H), 2.82 (t, J=4.8, 1H), 2.70-2.68 (dd, J=5.2, 2.8, 1H), 1.57 (s, 6H); ¹³C NMR (100 MHz, DMSO-d₆): δ 156.3, 156.0, 142.9, 142.6, 127.4, 127.4, 113.9, 80.3, 77.2, 71.0, 69.4, 68.8, 67.8, 57.9, 49.7, 43.8, 41.1, 30.7; HRMS (ESI) (m/z) calc'd for C₂₄H₂₈O₅Na [M+Na]⁺: 419.1834. found: 419.1840.

### Example 7

### Synthesis of (R)-1-chloro-3-(4-(2-(4-((S)-2-hydroxy-3-(prop-2-ynyloxy)propoxy)phenyl)propan-2-yl)phenoxy)propan-2-ol (10)

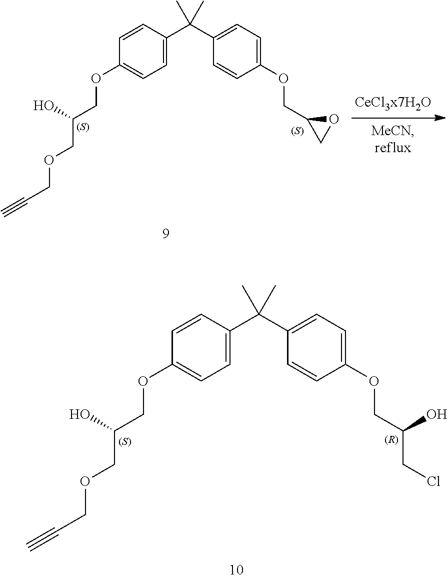

To a solution of derivative 9 (184 mg, 0.46 mmol, 1 equiv) in acetonitrile (3 mL) was added CeCl₃.7H₂O (260 mg, 0.69 mmol, 1.5 equiv), and the mixture was refluxed for 20 h. The resulting white paste was filtered and washed with ethyl acetate, and the clear suspension was concentrated under reduced pressure. The resulting residue was purified by flash column chromatography on silica gel Sep pak (5 g) (eluent: 30% ethyl acetate in hexane to 50% ethyl acetate) to provide 10 (91 mg, 45%) as a clear foam.

¹H NMR (400 MHz, CDCl₃): δ 7.17-7.13 (m, 4H), 6.85-6.81 (m, 4H), 4.23-4.19 (m, 4H), 4.09-4.06 (dd, J=4.8, 3.2, 2H), 4.04-4.02 (dd, J=5.2, 2.4, 2H), 3.81-3.67 (m, 4H), 2.57 (br, 2H), 2.47 (t, J=2.0, 1H), 1.65 (s, 6H); ¹³C NMR (100 MHz, CDCl₃): δ 156.5, 156.2, 144.1, 143.7, 128.0, 127.9, 114.2, 114.1, 79.5, 75.1, 71.0, 70.1, 69.2, 69.0, 68.6, 58.9, 46.1, 41.9, 31.2; HRMS (ESI) (m/z): calc'd for C₂₄H₂₉O₅NaCl [M+Na]⁺: 455.1601. found: 455.1609.

### Example 8

### Synthesis of (S)-1-(4-(2-(4-((R)-oxiran-2-Ylmethoxy)phenyl)propan-2-ylphenoxy)-3-(prop-2-ynyloxy)propan-2-ol (11)

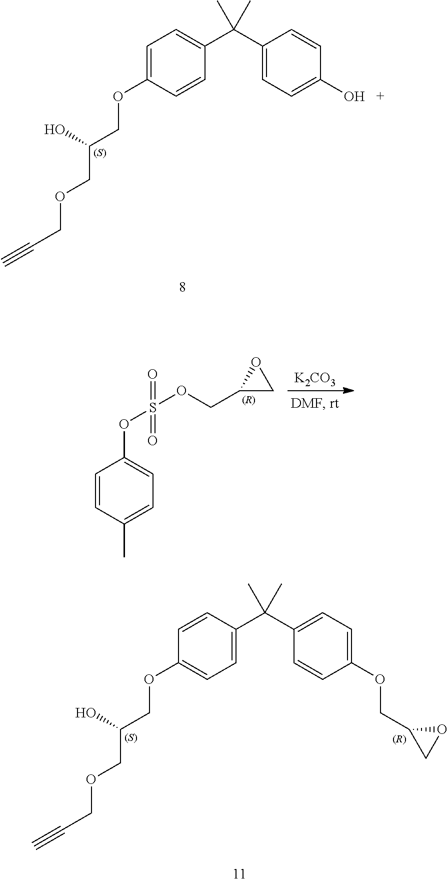

Potassium carbonate anhydrous (162 mg, 1.17 mmol, 2.0 equiv) was added to a stirred solution of derivative 8 (200 mg, 0.58 mmol, 1 equiv) in anhydrous dimethyl formamide (1.5 mL) at room temperature, and the contents were stirred under an atmosphere of argon for 20 min. A solution of (2R)-(−)-glycidyl tosylate 98% (267 mg, 1.17 mmol, 2.0 equiv) in anhydrous dimethyl formamide (1.5 mL) was added slowly via syringe, and the mixture was allowed to react at room temperature for 182 h. Then, the reaction was quenched by the addition of a saturated solution of ammonium chloride (1 mL), and the mixture was extracted with ethyl acetate (3×5 mL). The organic layer was washed with deionized water (5 mL), dried over anhydrous magnesium sulfate, filtered and concentrated under reduced pressure. The resulting residue was purified by flash column chromatography on silica gel (eluent: 10% ethyl acetate in hexane to 40% ethyl acetate) to provide 11 (6S,24R) (210 mg, 90%) as a clear foam.

### Example 9

### Synthesis of (S)-1-chloro-3-(4-(2-(4-((S)-2-hydroxy-3-(prop-2-ynyloxy)propoxy)phenyl)propan-2-yl)phenoxypropan-2-ol (12)

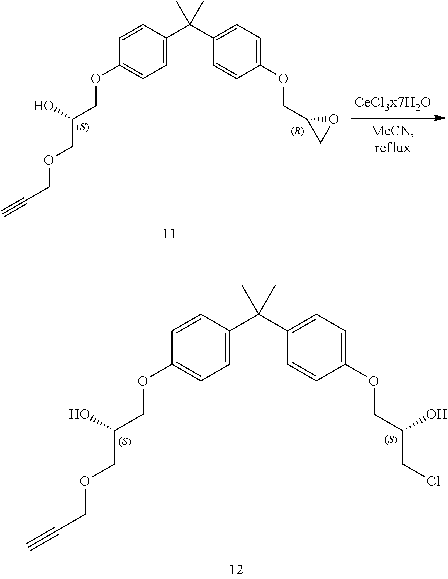

To a solution of derivative 11 (200 mg, 0.50 mmol, 1 equiv) in acetonitrile (3 mL) was added CeCl₃.7H₂O (282 mg, 0.75 mmol, 1.5 equiv), and the mixture was refluxed for 27 h. The resulting white paste was filtered and washed with ethyl acetate, and the clear suspension was concentrated under reduced pressure. The resulting residue was purified by flash column chromatography on silica gel Sep pak (5 g) (eluent: 10% ethyl acetate in hexane to 50% ethyl acetate) to provide 12 (6S,24S) (143 mg, 66%) as a clear foam. ¹H NMR (400 MHz, CDCl₃): δ 7.16-7.13 (m, 4H), 6.84-6.81 (m, 4H), 4.23-4.18 (m, 4H), 4.09-4.06 (dd, J=4.8, 3.2, 2H), 4.04-4.02 (dd, J=4.8, 2.0, 2H), 3.81-3.67 (m, 4H), 2.57 (br, 2H), 2.47 (t, J=2.4, 1H), 1.65 (s, 6H); ¹³C NMR (100 MHz, CDCl₃): δ 156.5, 156.2, 144.1, 143.7, 128.0, 127.9, 114.2, 114.1, 79.5, 75.1, 71.0, 70.1, 69.3, 69.0, 68.6, 58.9, 46.2, 41.9, 31.2; HRMS (ESI) (m/z): calc'd for C₂₄H₂₉O₅NaCl [M+Na]⁺: 455.1601. found: 455.1610.

### Example 10

### Synthesis of (R)-4-(2-(4-(OXIRAN-2-ylmethoxy)phenyl)propan-2-yl)phenol (13)

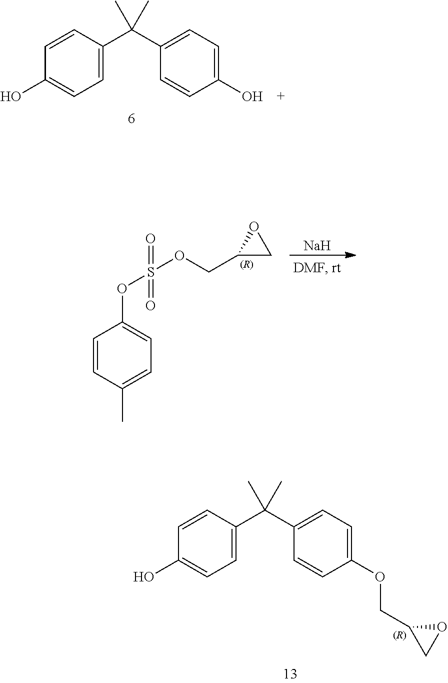

Sodium hydride (60% dispersion in mineral oil, 175 mg, 4.38 mmol, 1.0 equiv) was added slowly to a stirred solution of Bisphenol A 6 (1000 mg, 4.38 mmol, 1 equiv) in anhydrous dimethyl formamide (4 mL) at room temperature, and the contents were stirred under an atmosphere of argon for 20 min. A solution of (2R)-(−)-glycidyl tosylate 98% (1200 mg, 5.25 mmol, 1.2 equiv) in anhydrous dimethyl formamide (4 mL) was added slowly via syringe, and the mixture was allowed to react at room temperature for 15 h. Then, the reaction was quenched by the addition of a saturated solution of ammonium chloride (5 mL), and the mixture was extracted with ethyl acetate (3×15 mL). The organic layer was washed with deionized water (15 mL), dried over anhydrous magnesium sulfate, filtered and concentrated under reduced pressure. The resulting residue was purified by flash column chromatography on silica gel (eluent: 10% ethyl acetate in hexane) to provide 13 (587 mg, 57%) as a clear foam. Note: The same reaction yielded the bis epoxide analogue together with unreacted starting material. ¹H NMR (400 MHz, DMSO-d₆): δ 9.12 (s, 1H), 7.09 (d, J=8.8, 2H), 6.97 (d, J=8.8, 2H), 6.83 (d, J=8.8, 2H), 6.63 (d, J=8.4, 2H), 4.27-4.23 (dd, J=11.6, 2.8, 1H), 3.80-3.76 (dd, J=11.2, 6.4, 1H), 3.30-3.27 (m, 1H), 2.81 (t, J=4.8, 1H), 2.69-2.67 (dd, J=5.2, 2.8, 1H), 1.54 (s, 6H); ¹³C NMR (100 MHz, DMSO-d₆): δ 156.5, 155.6, 143.8, 141.3, 128.0, 127.9, 115.2, 114.4, 69.5, 50.4, 44.4, 41.7, 31.4; HRMS (ESI) (m/z): calc'd for C₁₈H₁₉O₃ [M-H]⁺: 283.1334. found: 283.1331.

### Example 11

### Synthesis of (R)-4-(2-(4-(2-hydroxy-3-(prop-2-ynyloxy)propoxy)phenyl)propan-2-yl)phenol (14)

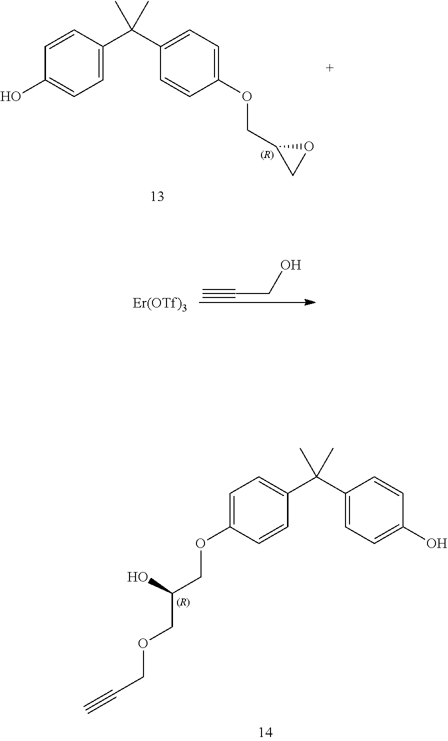

Propargyl alcohol (5.0 mL) was added to derivative 13 (587 mg, 2.06 mmol, 1 equiv), and the mixture was stirred for 10 min. Solid Erbium(III) trifluoromethanesulfonate (253 mg, 0.41 mmol, 1/5 equiv) was added in one portion, and the solution was stirred at room temperature for 24 h before being quenched with Sodium bicarbonate (2 mL). The reaction was then extracted with dichloromethane (3×5 mL). The organic layer was washed with deionized water (5 mL), dried over anhydrous magnesium sulfate, filtered and concentrated under reduced pressure. The resulting residue was purified by flash column chromatography on silica gel (eluent: 20% ethyl acetate in hexane to 40% ethyl acetate) to provide 14 (392 mg, 56%) as a pale foam.

### Example 12

### Synthesis of (R)-1-(4-(2-(4-((R)-oxiran-2-ylmethoxy)phenyl)propan-2-yl)phenoxy)-3-(prop-2-ynyloxy)propan-2-ol (15)

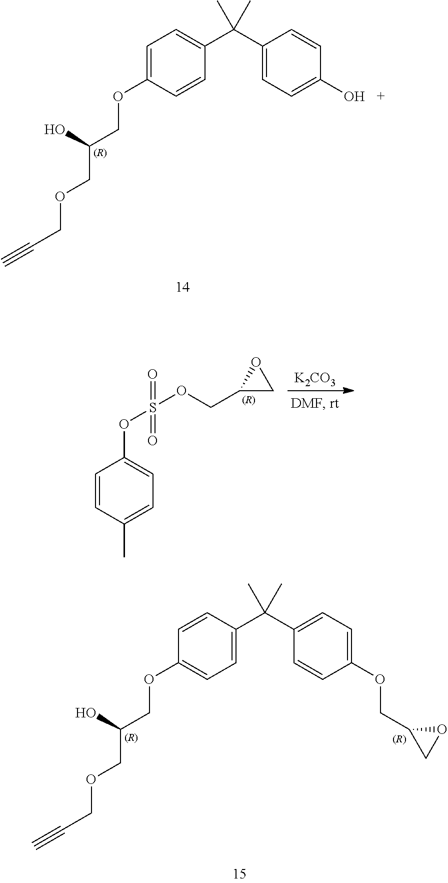

Potassium carbonate anhydrous (221 mg, 1.60 mmol, 2.0 equiv) was added to a stirred solution of derivative 14 (272 mg, 0.80 mmol, 1 equiv) in anhydrous dimethyl formamide (3 mL) at room temperature, and the contents were stirred under an atmosphere of argon for 20 min. A solution of (2R)-(−)-glycidyl tosylate 98% (365 mg, 1.60 mmol, 2.0 equiv) in anhydrous dimethyl formamide (2 mL) was added slowly via syringe, and the mixture was allowed to react at room temperature for 192 h. Then, the reaction was quenched by the addition of a saturated solution of ammonium chloride (2 mL), and the mixture was extracted with ethyl acetate (3×10 mL). The organic layer was washed with deionized water (10 mL), dried over anhydrous magnesium sulfate, filtered and concentrated under reduced pressure. The resulting residue was purified by flash column chromatography on silica gel (eluent: 10% ethyl acetate in hexane to 40% ethyl acetate) to provide 15 (191 mg, 60%) as a clear foam.

### Example 13

### Synthesis of (S)-1-chloro-3-(4-(2-(4-((R)-2-hydroxy-3-(prop-2-ynyloxy)propoxy)phenylpropan-2-yl)phenoxy)propan-2-ol (16)

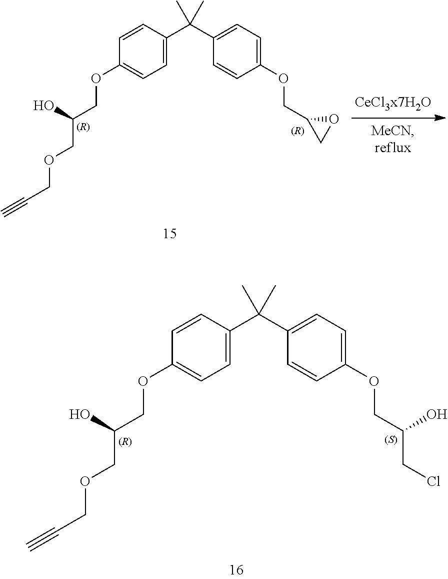

To a solution of derivative 15 (191 mg, 0.48 mmol, 1 equiv) in acetonitrile (3 mL) was added CeCl₃.7H₂O (269 mg, 0.72 mmol, 1.5 equiv), and the mixture was refluxed for 27 h. The resulting white paste was filtered and washed with ethyl acetate, and the clear suspension was concentrated under reduced pressure. The resulting residue was purified by flash column chromatography on silica gel Sep pak (5 g) (eluent: 30% ethyl acetate in hexane) to provide 16 (178 mg, 86%) as a clear foam. ¹H NMR (400 MHz, CDCl₃): δ 7.13-7.10 (dd, J=8.8, 2.8, 4H), 6.80 (d, J=8.4, 4H), 4.20-4.16 (m, 4H), 4.05-4.02 (m, 2H), 4.00-3.99 (m, 2H), 3.78-3.64 (m, 4H), 3.11 (br, 2H), 2.45 (t, J=2.4, 1H), 1.62 (s, 6H); ¹³C NMR (100 MHz, CDCl₃): δ 156.5, 156.2, 143.8, 143.5, 127.9, 127.8, 114.1, 114.0, 79.5, 75.0, 71.0, 69.8, 69.1, 69.0, 68.6, 58.8, 46.2, 41.8, 31.1; HRMS (ESI) (m/z): calc'd for C₂₄H₂₉O₅NaCl [M+Na]⁺: 455.1601. found: 455.1595.

### Example 14

### Synthesis of (R)-1-(4-(2-(4-((S)-oxiran-2-ylmethoxy)phenyl)propan-2-yl)phenoxy)-3-(prop-2-ynyloxy)propan-2-ol (17)

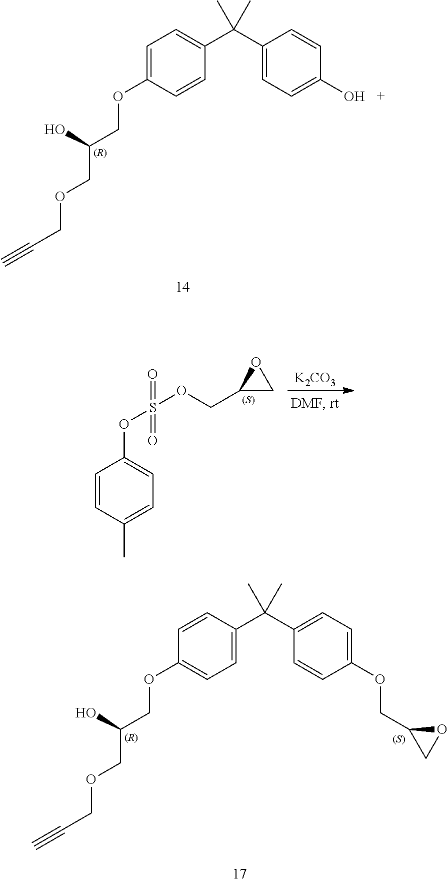

Potassium carbonate anhydrous (97 mg, 0.70 mmol, 2.0 equiv) was added to a stirred solution of derivative 14 (120 mg, 0.35 mmol, 1 equiv) in anhydrous dimethyl formamide (1.5 mL) at room temperature, and the contents were stirred under an atmosphere of argon for 20 min. A solution of (2S)-(+)-glycidyl tosylate 98% (160 mg, 0.70 mmol, 2.0 equiv) in anhydrous dimethyl formamide (1.5 mL) was added slowly via syringe, and the mixture was allowed to react at room temperature for 192 h. Then, the reaction was quenched by the addition of a saturated solution of ammonium chloride (1 mL), and the mixture was extracted with ethyl acetate (3×5 mL). The organic layer was washed with deionized water (5 mL), dried over anhydrous magnesium sulfate, filtered and concentrated under reduced pressure. The resulting residue was purified by flash column chromatography on silica gel (eluent: 20% ethyl acetate in hexane) to provide 17 (116 mg, 83%) as a clear foam.

### Example 15

### Synthesis of (R)-1-chloro-3-(4-(2-(4-((R)-2-hydroxy-3-(prop-2-ynyloxy)propoxy)phenyl)propan-2-ylphenoxy)propan-2-ol (18)

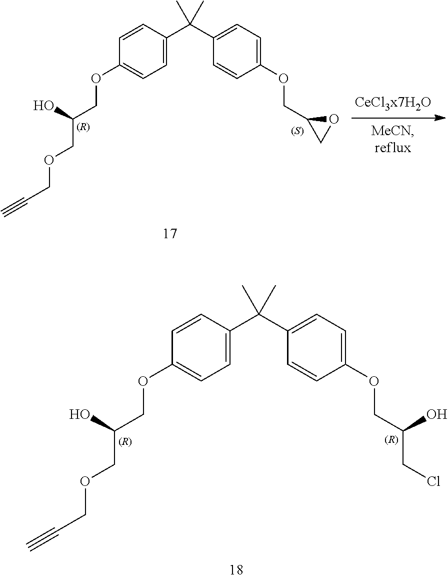

To a solution of derivative 17 (116 mg, 0.29 mmol, 1 equiv) in acetonitrile (3 mL) was added CeCl₃.7H₂O (163 mg, 0.44 mmol, 1.5 equiv), and the mixture was refluxed for 22 h. The resulting white paste was filtered and washed with ethyl acetate, and the clear suspension was concentrated under reduced pressure. The resulting residue was purified by flash column chromatography on silica gel Sep pak (5 g) (eluent: 10% ethyl acetate in hexane to 30% ethyl acetate) to provide 18 (47 mg, 37%) as a clear foam.

¹H NMR (400 MHz, CDCl₃): δ 7.16-7.13 (m, 4H), 6.84-6.82 (m, 4H), 4.23-4.17 (m, 4H), 4.08-4.05 (m, 2H), 4.04-4.00 (m, 2H), 3.81-3.67 (m, 4H), 2.61 (br, 2H), 2.47-2.46 (m, 1H), 1.65 (s, 6H); ¹³C NMR (100 MHz, CDCl₃): δ 156.5, 156.2, 144.1, 143.7, 128.0, 127.9, 114.2, 114.1, 79.5, 75.1, 71.0, 70.1, 69.3, 69.0, 68.6, 58.9, 46.2, 41.9, 31.2; HRMS (ESI) (m/z): calc'd for C₂₄H₂₉O₅NaCl [M+Na]⁺: 455.1601. found: 455.1605.

### Example 16

### Synthesis of 3,3′-(4,4′-(propane-2,2-diyl)bis(4,1-phenylene))bis(oxy)bis(1-methoxypropan-2-ol) (28)

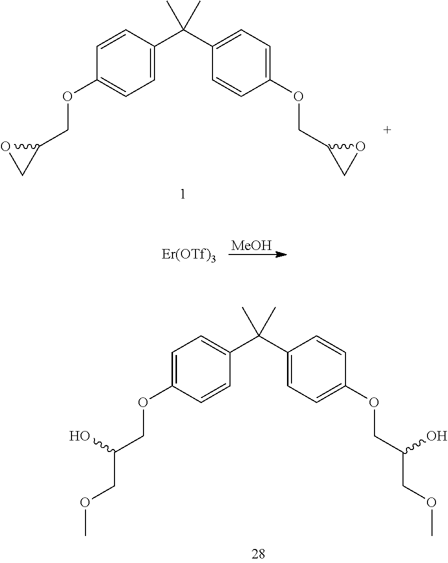

To a solution of racemic 1 (32 mg, 0.094 mmol, 1 equiv) in methanol (0.3 mL) was added solid erbium(III) trifluoromethanesulfonate (58 mg, 0.094 mmol, 1 equiv) in portions over an hour, and the mixture was stirred at room temperature for 6 h. The organic solvent was evaporated under a stream of nitrogen, and the residue was purified by flash column chromatography on silica gel Sep pak (10 g) (eluent: 5% methanol in dichloromethane) to provide 28 (31 mg, 82%) as a colourless solid. ¹H NMR (400 MHz, DMSO-d₆): δ 7.08 (d, J=8.8, 4H), 6.80 (d, J=8.8, 4H), 5.06 (d, J=5.2, 2H), 3.92-3.87 (m, 4H), 3.84-3.81 (m, 2H), 3.42-3.35 (m, 4H), 3.25 (s, 6H), 1.56 (s, 6H); ¹³C NMR (100 MHz, DMSO-d₆): δ 157.0, 143.2, 128.0, 114.4, 74.4, 70.1, 68.4, 50.1, 41.7, 31.3; HRMS (ESI) (m/z): calc'd for C₂₃H₃₂O₆Na [M+Na]⁺: 427.2097. found: 427.2087.

### Example 17

### Synthesis of 1-methoxy-3-(4-(2-(4-(oxiran-2-ylmethoxy)phenyl)propan-2-yl)phenoxy)propan-2-ol (29)

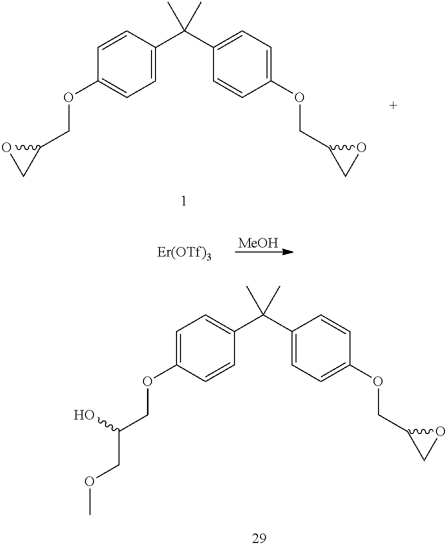 [SMILES: "MeOH" ; CC(C)(c1ccc(OCC2CO2)cc1)c1ccc(OCC2CO2)cc1 ; COCC(O)COc1ccc(C(C)(C)c2ccc(OCC3CO3)cc2)cc1 ; "Er(OTf)3"]

To a solution of 1 (500 mg, 1.46 mmol, 1 equiv) in methanol (5 mL) was added solid erbium(III) trifluoromethanesulfonate (90 mg, 0.146 mmol, 1/10 equiv) in one portion, and the mixture was stirred at room temperature for 1 h. Sodium bicarbonate was added (1 mL), the organic solvent was evaporated under reduced pressure, and the residue was extracted with dichloromethane (3×5 mL). The organic layer was washed with deionized water (2×5 mL), dried over anhydrous magnesium sulfate, filtered and concentrated under reduced pressure. The resulting residue was purified by flash column chromatography on silica gel (eluent: 10% to 40% ethyl acetate in hexane) to provide 29 (128 mg, 23%) as a pale foam. ¹H NMR (400 MHz, DMSO-d₆): δ 7.12-7.07 (m, 4H), 6.86-6.80 (m, 4H), 5.07 (s, 1H), 4.27-4.24 (dd, J=11.2, 2.4, 1H), 3.91-3.88 (m, 2H), 3.85-3.76 (m, 2H), 3.43-3.34 (m, 2H), 3.31-3.28 (m, 1H), 3.27 (m, 3H), 2.83-2.81 (dd, J=4.8, 4.0, 1H), 2.69-2.67 (dd, J=5.2, 2.8, 1H), 1.56 (s, 6H); ¹³C NMR (100 MHz, DMSO-d₆): δ 157.0, 156.6, 143.6, 143.2, 128.0, 128.0, 114.5, 114.5, 74.5, 70.1, 69.5, 68.5, 59.1, 50.4, 44.4, 41.8, 31.3; HRMS (ESI) (m/z): calc'd for C₂₂H₂₈O₅Na [M+Na]⁺: 395.1834. found: 395.1839.

### Example 18

### Synthesis of 1-chloro-3-(4-(2-(4-(2-hydroxy-3-methoxypropoxy)phenyl)propan-2-yl)phenoxy)propan-2-ol (30)

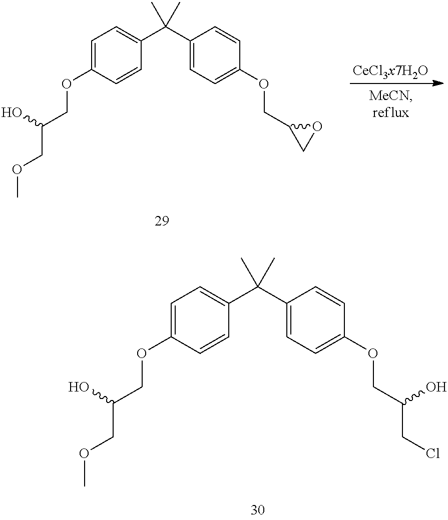 [SMILES: COCC(O)COc1ccc(C(C)(C)c2ccc(OCC3CO3)cc2)cc1 ; COCC(O)COc1ccc(C(C)(C)c2ccc(OCC(O)CCl)cc2)cc1]

To a solution of racemic 29 (64 mg, 0.17 mmol, 1 equiv) in acetonitrile (2 mL) was added CeCl₃.7H₂O (96 mg, 0.25 mmol, 1.5 equiv), and the mixture was refluxed for 17 h. The resulting white paste was filtered and washed with ethyl acetate, and the clear suspension was concentrated under reduced pressure. The resulting residue was purified by flash column chromatography on silica gel (eluent: 40% ethyl acetate in hexane) to provide 30 (70 mg, 99%) as a pale foam. ¹H NMR (400 MHz, DMSO-d₆): δ 7.11-7.08 (dd, J=8.8, 4.0, 4H), 6.83 (t, J=8.4, 4H), 5.52 (d, J=4.8, 1H), 5.06 (d, J=4.8, 1H), 4.04-4.00 (m, 1H), 3.95-3.89 (m, 4H), 3.86-3.81 (m, 1H), 3.76-3.72 (dd, J=11.2, 4.4, 1H), 3.68-3.64 (dd, J=11.2, 5.6, 1H), 3.44-3.36 (m, 2H), 3.27 (s, 3H), 1.58 (s, 6H);

¹³C NMR (100 MHz, DMSO-d₆): δ 157.0, 156.7, 143.5, 143.2, 128.0, 128.0, 114.5, 114.5, 74.5, 70.1, 69.5, 69.3, 68.5, 59.1, 47.4, 41.8, 31.3; HRMS (ESI) (m/z): calc'd for C₂₂H₂₉O₅NaCl [M+Na]⁺: 431.1601. found: 431.1605.

### Example 19

### Synthesis of 3,3′-(4,4′-(propane-2,2-diyl)bis(4,1-phenylene))bis(oxy)bis(1-isopropoxypropan-2-ol) (31)

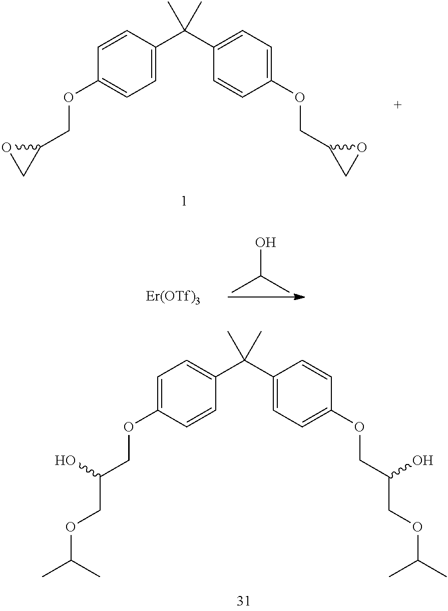

To a solution of racemic 1 (1.02 g, 2.99 mmol, 1 equiv) in 2-propanol (5 mL) was added solid erbium(III) trifluoromethanesulfonate (183 mg, 0.299 mmol, 1/10 equiv) in one portion, and the mixture was stirred at room temperature for 2 h. Sodium bicarbonate was added (2 mL), the organic solvent was evaporated under reduced pressure, and the residue was extracted with dichloromethane (3×5 mL). The organic layer was washed with deionized water (2×5 mL), dried over anhydrous magnesium sulfate, filtered and concentrated under reduced pressure. The resulting residue was purified by flash column chromatography on silica gel (eluent: 0% to 5% methanol in dichloromethane) to provide 31 (1003 mg, 75%) as a pale foam. ¹H NMR (400 MHz, DMSO-d₆): δ 7.09 (d, J=8.8, 4H), 6.82 (d, J=8.8, 4H), 5.00 (br, 2H), 3.93-3.82 (m, 6H), 3.57-3.51 (m, 2H), 3.45-3.38 (m, 4H), 1.57 (s, 6H), 1.07 (d, J=6.0, 12H); ¹³C NMR (100 MHz, DMSO-d₆): δ 156.5, 142.6, 127.4, 113.8, 71.2, 69.6, 69.2, 68.3, 41.1, 30.7, 22.0; HRMS (ESI) (m/z): calc'd for C₂₇H₃₉O₆Na [M+Na]⁺: 459.2747. found: 459.2757.

### Example 20

### Synthesis of 1-chloro-3-(4-(2-(4-(2-hydroxy-3-isopropdxypropoxy)phenyl)propan-2-yl)phenoxy)propan-2-ol (32)

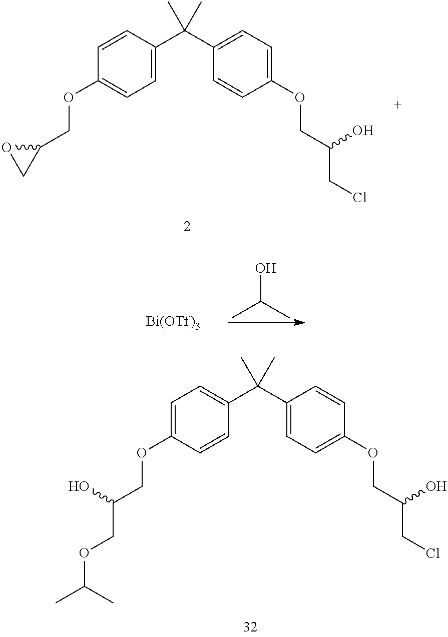

To a solution of racemic 2 (50 mg, 0.13 mmol, 1 equiv) in 2-propanol (1.5 mL) was added solid bismuth(III) trifluoromethanesulfonate (4 mg, 0.0065 mmol, 1/20 equiv) in one portion, and the mixture was stirred at room temperature for 12 h. Sodium bicarbonate was added (0.5 mL), the organic solvent was evaporated under reduced pressure, and the residue was extracted with dichloromethane (3×3 mL). The organic layer was washed with deionized water (2×3 mL), dried over anhydrous magnesium sulfate, filtered and concentrated under reduced pressure. The resulting residue was purified by flash column chromatography on silica gel (eluent: 40% to 80% ethyl acetate in hexane) to provide 32 (41 mg, 70%) as a foam. ¹H NMR (400 MHz, DMSO-d₆): δ 7.11-7.08 (dd, J=8.4, 3.6, 4H), 6.84-6.81 (dd, J=8.4, 6.8, 4H), 5.52 (br, 1H), 4.99 (br, 1H), 4.00 (br, 1H), 3.93-3.88 (m, 3H), 3.84-3.80 (m, 2H), 3.76-3.72 (dd, J=15.2, 8.8, 1H), 3.67-3.63 (dd, J=11.2, 5.2, 1H), 3.57-3.51 (m, 1H), 3.39 (t, J=4.8, 2H), 1.67 (s, 6H), 1.07 (d, J=6.0, 6H); ¹³C NMR (100 MHz, DMSO-d₆): δ 156.5, 156.1, 142.9, 142.5, 127.4, 127.4, 113.9, 71.2, 69.6, 69.1, 68.8, 68.6, 68.3, 46.8, 41.1, 30.7, 22.0; HRMS (ESI) (m/z): calc'd for C₂₄H₃₃O₅NaCl [M+Na]⁺: 459.1914. found: 459.1910.

### Example 21

### Synthesis of 3,3′-(4,4′-(propane-2,2-diyl)bis(4,1-phenylene))bis(oxy)bis(1-butoxypropan-2-ol) (33)

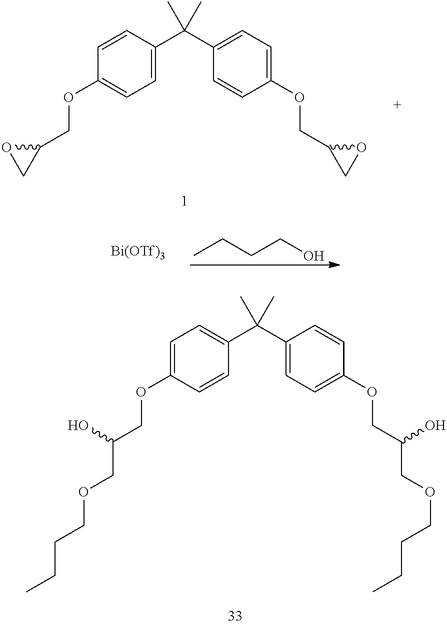

Compound 33 was prepared using a procedure analogous to that described in Example 19 using n-butanol in place of isopropanol. ¹H NMR (400 MHz, DMSO-d₆): δ 7.08 (d, J=8.4, 4H), 6.80 (d, J=8.8, 4H), 5.03 (br, 2H), 3.92-3.82 (m, 6H), 3.42-3.32 (m, 8H), 1.57 (s, 6H), 1.49-1.42 (m, 4H), 1.34-1.25 (m, 4H); ¹³C NMR (100 MHz, DMSO-d₆): δ 156.4, 142.6, 127.4, 113.8, 71.8, 70.3, 69.5, 68.0, 41.1, 31.2, 30.7, 18.8, 13.8; HRMS (ESI) (m/z): calc'd for C₂₉H₄₄O₆Na [M+Na]⁺: 511.3036. found: 511.3039.

### Example 22

### Synthesis of 1-butoxy-3-(4-(2-(4-(3-chloro-2-hydroxypropoxy)phenyl)propan-2-yl)phenoxy)propan-2-ol (34)

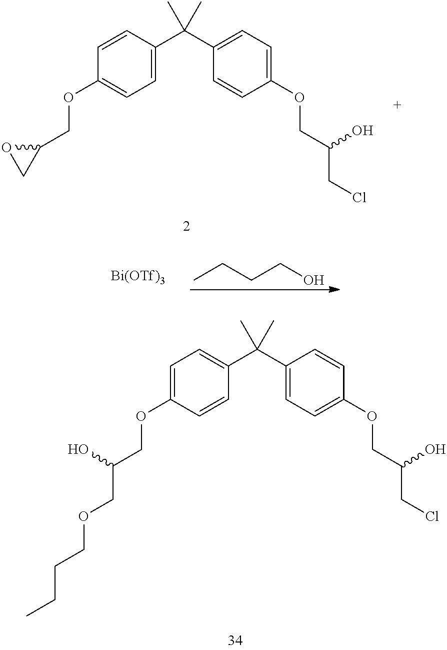

To a solution of racemic 2 (50 mg, 0.13 mmol, 1 equiv) in n-butanol (1.5 mL) was added solid bismuth(III) trifluoromethanesulfonate (4 mg, 0.0065 mmol, 1/20 equiv) in one portion, and the mixture was stirred at room temperature for 12 h. Sodium bicarbonate was added (0.5 mL), solvents were evaporated under reduced pressure, and the residue was extracted with dichloromethane (3×3 mL). The organic layer was washed with deionized water (2×3 mL), dried over anhydrous magnesium sulfate, filtered and concentrated under reduced pressure. The resulting residue was purified by flash column chromatography on silica gel (eluent: 50% ethyl acetate in hexane) to provide 34 (44 mg, 74%) as a foam. ¹H NMR (400 MHz, DMSO-d₆): δ 7.11-7.08 (dd, J=8.8, 3.6, 4H), 6.82 (t, J=8.0, 4H), 5.52 (br, 1H), 5.03 (br, 1H), 4.04-3.99 (m, 1H), 3.94-3.86 (m, 4H), 3.85-3.83 (m, 1H), 3.76-3.72 (dd, J=11.2, 4.8, 1H), 3.67-3.63 (dd, J=10.8, 5.2, 1H), 3.45-3.37 (m, 4H), 1.57 (s, 6H), 1.50-1.43 (m, 2H), 1.34-1.25 (m, 2H), 0.86 (t, J=3.6, 1H); ¹³C NMR (100 MHz, DMSO-d₆): δ 156.4, 156.1, 142.9, 142.5, 127.4, 127.4, 113.9, 113.8, 71.8, 70.3, 69.5, 68.8, 68.6, 68.0, 46.8, 41.1, 31.3, 30.7, 18.8, 13.8; HRMS (ESI) (m/z): calc'd for C₂₅H₃₅O₅NaCl [M+Na]⁺: 473.2071. found: 473.2077.

### Example 23

### Synthesis of 1-(3-chloropropoxy)-4-(2-(4-(2-(2-(2-(prop-2-ynyloxy)ethoxy)ethoxy)ethoxy)phenyl)propan-2-yl)benzene (40)

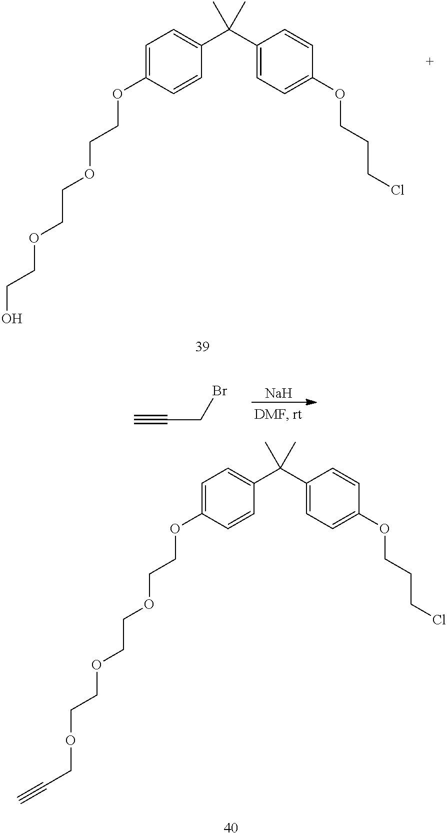

Sodium hydride (60% dispersion in mineral oil, 2 mg, 0.052 mmol, 1.2 equiv) was added slowly to a stirred solution of 39 (19 mg, 0.043 mmol, 1 equiv) in anhydrous dimethyl formamide (1 mL) at room temperature, and the contents were stirred under an atmosphere of argon for 5 min. Propargyl bromide (12 μL, 0.129 mmol, 3 equiv) was added via syringe, and the mixture was allowed to react at rt for 5 h. Deionized water (0.5 mL) was added, and the mixture was extracted with ethyl acetate (3×3 mL). The organic layer was washed with deionized water (3 mL), dried over anhydrous magnesium sulfate, filtered and concentrated under reduced pressure. The resulting residue was purified by flash column chromatography on silica gel (eluent: 40% ethyl acetate in hexane) to provide 40 (17 mg, 80%) as a foam. ¹H NMR (400 MHz, CDCl₃): δ 7.12-7.09 (m, 4H), 6.80-6.77 (m, 4H), 4.18 (d, J=2.4, 2H), 4.10-4.05 (m, 4H), 3.82 (t, J=5.2, 2H), 3.73-3.65 (m, 10H), 2.39 (t, J=2.4, 1H), 2.23-2.15 (m, 2H), 1.61 (s, 6H); ¹³C NMR (100 MHz, CDCl₃): δ 156.9, 156.2, 144.3, 143.5, 128.0, 127.9, 114.2, 114.1, 78.8, 74.7, 71.0, 70.9, 70.7, 70.0, 69.4, 67.6, 64.4, 58.6, 41.9, 41.8, 32.6, 31.3; HRMS (ESI) (m/z): calc'd for C₂₇H₃₅O₅NaCl [M+Na]⁺: 497.2071. found: 497.2064.

### Example 24

### Synthesis of (R)-2-(2-(2-(4-(2-(4-(oxiran-2-ylmethoxy)phenyl)propan-2-yl)phenoxy)ethoxy)ethoxy)ethanol (42)

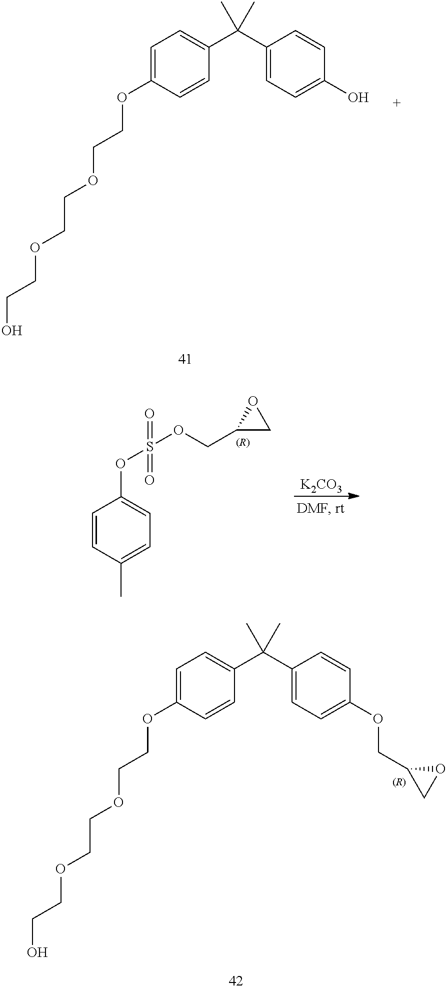

Potassium carbonate anhydrous (69 mg, 0.50 mmol, 3 equiv) was added to a stirred solution of 41 (60 mg, 0.17 mmol, 1 equiv) in anhydrous dimethyl formamide (2 mL) at room temperature, and the contents were stirred under an atmosphere of argon for 20 min. A solution of (2R)-(−)-glycidyl tosylate 98% (114 mg, 0.50 mmol, 3 equiv) in anhydrous dimethyl formamide (1 mL) was added slowly via syringe, and the mixture was allowed to react at room temperature for 97 h. Then, the reaction was quenched by the addition of a saturated solution of ammonium chloride (1 mL), and the mixture was extracted with ethyl acetate (3×5 mL). The organic layer was washed with deionized water (5 mL), dried over anhydrous magnesium sulfate, filtered and concentrated under reduced pressure. The resulting residue was purified by flash column chromatography on silica gel (eluent: 0% to 5% methanol in dichloromethane) to provide 42 (25R) (49 mg, 70%) as a pale foam. ¹H NMR (400 MHz, DMSO-d₆): δ 7.11-7.08 (dd, J=8.8, 4.4, 4H), 6.83 (t, J=8.4, 4H), 4.55 (br, 1H), 4.28-4.25 (dd, J=11.2, 2.4, 1H), 4.04 (t, J=4.4, 2H), 3.81-3.77 (dd, J=11.6, 6.8, 1H), 3.72 (t, J=4.8, 2H), 3.59-3.57 (m, 2H), 3.54-3.52 (m, 2H), 3.50-3.47 (m, 2H), 3.42 (t, J=5.2, 2H), 3.32-3.29 (m, 1H), 2.82 (t, J=4.8, 1H), 2.70-2.68 (dd, J=4.8, 2.4, 1H), 1.57 (s, 6H); ¹³C NMR (100 MHz, DMSO-d₆): δ 156.2, 156.0, 142.9, 142.6, 127.4, 127.4, 113.9, 113.8, 72.3, 69.9, 69.8, 69.0, 68.8, 67.0, 60.2, 49.7, 43.7, 41.1, 30.7; HRMS (ESI) (m/z): calc'd for C₂₄H₃₂O₆Na [M+Na]⁺: 439.2097. found: 439.2092.

### Example 25

### Synthesis of (S)-1-chloro-3-(4-(2-(4-(2-(2-(2-(prop-2-ynyloxy)ethoxy)ethoxy)ethoxyphenyl)propan-2-yl)phenoxy)propan-2-ol (43)

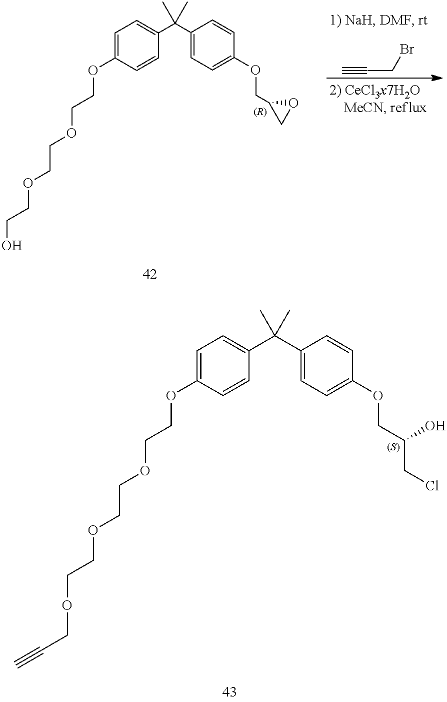

Sodium hydride (60% dispersion in mineral oil, 1 mg, 0.031 mmol, 1.3 equiv) was added slowly to a stirred solution of 42 (10 mg, 0.024 mmol, 1 equiv) in anhydrous dimethyl formamide (1 mL) at room temperature, and the contents were stirred under an atmosphere of argon for 5 min. Propargyl bromide (11 μL, 0.12 mmol, 5 equiv) was added via syringe, and the mixture was allowed to react at rt for 14 h. Water was added (2 mL), and the reaction was concentrated to dryness under reduced pressure and filtered over silica gel (2% methanol in dichloromethane). The crude residue was dissolved in acetonitrile (1 mL), and solid CeCl₃.7H₂O (13 mg, 0.036 mmol) was added in one portion. The mixture was refluxed for 12 h. The resulting white paste was filtered and washed with ethyl acetate, and the clear suspension was concentrated under reduced pressure. The resulting residue was purified by flash column chromatography on silica gel (eluent: 30% to 40% ethyl acetate in hexane) to provide 43 (10 mg, 85%, 2 steps) as a foam. ¹H NMR (400 MHz, DMSO-d₆): δ 7.13-7.09 (dd, J=10.4, 9.2, 4H), 6.79 (d, J=8.8, 4H), 4.18-4.16 (m, 3H), 4.08 (t, J=4.8, 2H), 4.05 (t, J=4.0, 2H), 3.82 (t, J=5.2, 2H), 3.78-3.74 (m, 1H), 3.72-3.70 (m, 3H), 3.67-3.65 (m, 6H), 2.48 (br, 1H), 2.39 (t, J=2.4, 1H), 1.61 (s, 6H); ¹³C NMR (100 MHz, CDCl₃): δ 156.9, 156.2, 144.3, 143.3, 128.1, 127.9, 114.2, 114.1, 74.7, 71.0, 70.9, 70.7, 70.1, 70.0, 69.3, 68.6, 67.6, 58.6, 46.2, 41.9, 31.2; HRMS (ESI) (m/z): calc'd for C₂₇H₃₅O₆NaCl [M+Na]⁺: 513.2020. found: 513.2028.

### Example 26

### Synthesis of (S)-1-chloro-3-(4-(2-(4-(2-(2-(2-hydroxyethoxy)ethoxy)ethoxy)phenyl)propan-2-yl)phenoxy)propan-2-ol (44)

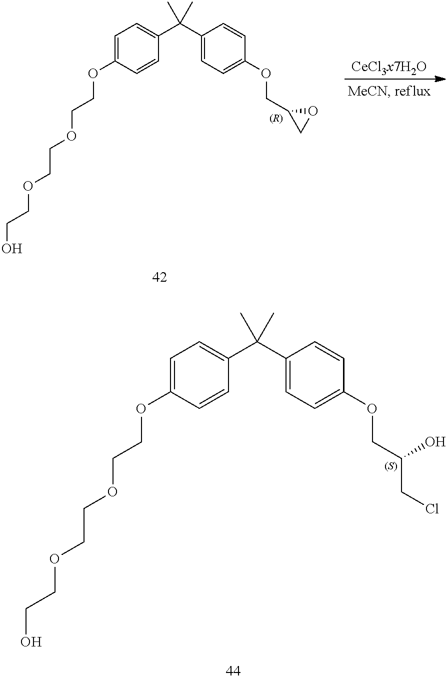

To a solution of 42 (7 mg, 0.017 mmol, 1 equiv) in acetonitrile (1 mL) was added CeCl₃.7H₂O (10 mg, 0.025 mmol, 1.5 equiv), and the mixture was refluxed for 14 h. The resulting white paste was filtered and washed with ethyl acetate, and the clear suspension was concentrated under reduced pressure. The resulting residue was purified by flash column chromatography on silica gel (eluent: 60% to 80% ethyl acetate in hexane) to provide 44 (6.8 mg, 89%) as a clear foam. ¹H NMR (400 MHz, CDCl₃): δ 7.11 (t, J=8.8, 4H), 6.79 (d, J=7.2, 4H), 4.19-4.15 (m, 1H), 4.09 (t, J=4.4, 2H), 4.03 (t, J=4.8, 2H), 3.83 (t, J=5.2, 2H), 3.78-3.67 (m, 8H), 3.60 (t, J=4.8, 2H), 1.90 (br, 2H), 1.61 (s, 6H); ¹³C NMR (100 MHz, CDCl₃): δ 156.9, 156.2, 144.3, 143.5, 128.1, 127.9, 114.2, 114.1, 72.7, 71.0, 70.6, 70.1, 70.0, 68.6, 67.5, 62.0, 46.2, 42.0, 31.2; HRMS (ESI) (m/z): calc'd for C₂₄H₃₃O₆NaCl [M+Na]⁺: 475.1863. found: 475.1870.

### Example 27

### Synthesis of (S)-1-chloro-3-(4-(2-(4-((R)-2-hydroxy-3-(2-(2-(2-hydroxyethoxy)ethoxy)ethoxy)propoxy)phenyl)propan-2-yl)phenoxy)propan-2-ol(46)

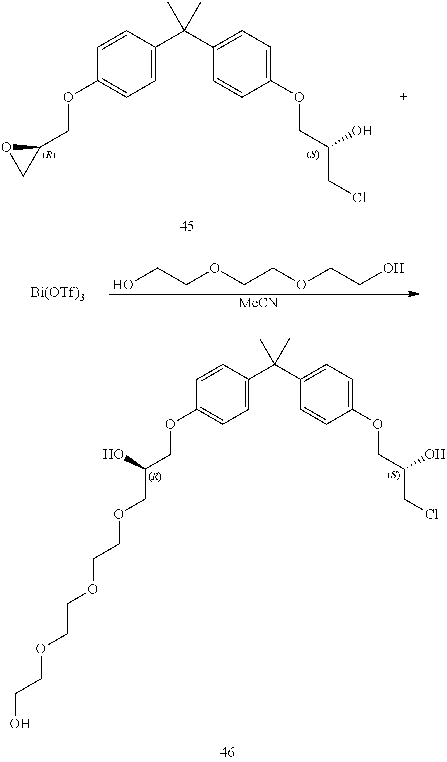

To a solution of 45 (35 mg, 0.092 mmol, 1 equiv) in acetonitrile (1 mL) was added triethylene glycol (1 mL) and bismuth(III) trifluoromethanesulfonate (12 mg, 0.018 mmol, 1/5 equiv), and the mixture was stirred at room temperature for 16 h. After the crude was concentrated under reduced pressure (to remove acetonitrile), the resulting residue was extracted with ethyl acetate/water (3×5 mL). The organic layer was washed with deionized water (5 mL), dried over anhydrous magnesium sulfate, filtered and concentrated under reduced pressure. The resulting residue was purified by flash column chromatography on silica gel (eluent: dichloromethane to 5% methanol in dichloromethane) to provide 46 (37 mg, 76%) as a pale foam. ¹H NMR (400 MHz, DMSO-d₆): δ 7.11-7.08 (dd, J=8.8, 4.0, 4H), 6.84-6.81 (dd, J=8.8, 6.8, 4H), 5.52 (d, J=5.2, 1H), 5.05 (d, J=4.8, 1H), 4.56 (t, J=5.2, 1H), 4.04-3.98 (m, 1H), 3.94-3.89 (m, 4H), 3.87-3.81 (m, 1H), 3.76-3.72 (dd, J=11.2, 4.8, 1H), 3.67-3.63 (dd, J=11.2, 5.6, 1H), 3.53-3.44 (m, 12H), 3.39 (t, J=5.2, 2H), 1.57 (s, 6H); ¹³C NMR (100 MHz, DMSO-d₆): δ 156.4, 156.1, 142.9, 142.5, 127.4, 127.4, 113.9, 113.8, 72.3, 72.2, 70.2, 69.8, 69.8, 69.7, 69.5, 68.8, 68.6, 68.0, 60.2, 46.8, 41.1, 30.7; HRMS (ESI) (m/z): calc'd for C₂₇H₃₉O₈NaCl [M+Na]⁺: 549.2231. found: 549.2220.

### Example 28

### Synthesis of 3,3′-(4,4′-(propane-2,2-diyl)bis(4,1-phenylene))bis(oxy)bis(1-(cyclohexyloxy)propan-2-ol) (47)

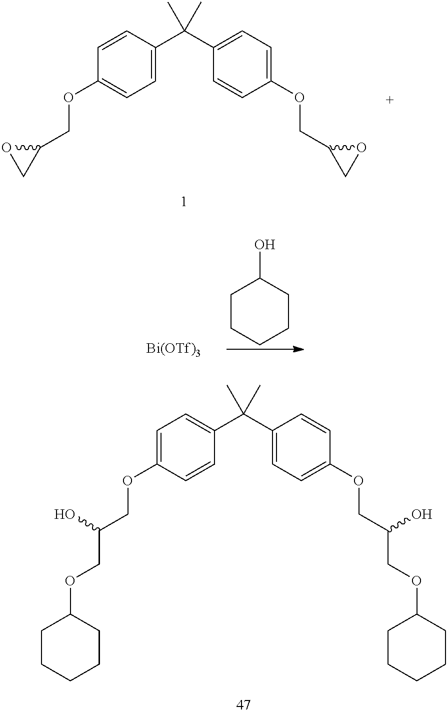

Compound 47 was prepared using a procedure analogous to that described in Example 19 using cyclohexanol in place of isopropanol. HRMS (ESI) (m/z): calc'd for C₃₃H₄₈O₆Na [M+Na]⁺: 563.3349. found: 563.3.

### Example 29

### Synthesis of 1-chloro-3-(4-(2-(4-(3-(cyclohexyloxy)-2-hydroxypropoxy)phenyl)propan-2-yl)phenoxy)propan-2-ol (48)

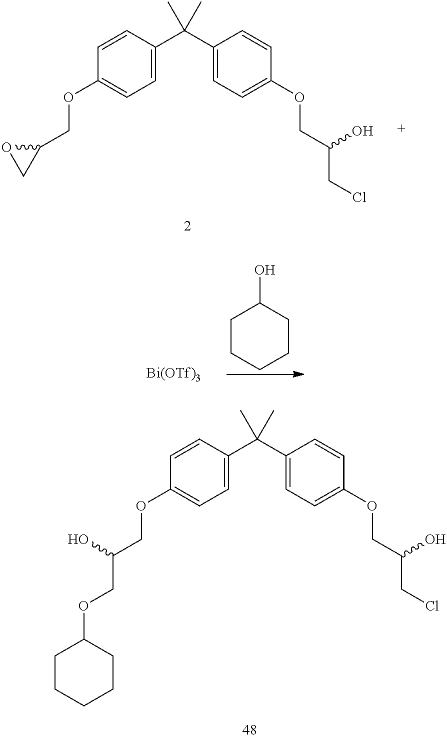

Compound 48 was prepared using a procedure analogous to that described in Example 20 using cyclohexanol in place of isopropanol. HRMS (ESI) (m/z): calc'd for C₂₇H₃₇O₅NaCl [M+Na]⁺: 499.2227. found: 499.2.

### Example 30

### Activity of Representative Compounds

LNCaP cells were transiently cotransfected with PSA (6.1 kb)-luciferase (0.25 μg/well) in 24-well plates for 24 h prior to pre-treatment with compounds for 1 hour before the addition of synthetic androgen, R1881 (1 nM), to induce PSA production or vehicle. The total amount of plasmid DNA transfected was normalized to 0.75 μg/well by the addition of the empty vector. After 48 h of incubation with R1881, the cells were harvested, and relative luciferase activity was determined. Test compounds were added to the cells at various concentrations and activity for each treatment was normalized to the predicted maximal activity induction (in the absence of test compounds, vehicle only). Plotting of sigmoidal curves (Boltzmann Function) and IC50 calculations were done using OriginPro 8.1 Sofware (Northampton, Mass., USA). Furthermore, toxicity was assessed by both microscopic examination and reduction of protein levels. Solubility was assessed both macroscopically (cloudy media) and microscopically (formation of granules or crystals). TABLE 3 shows activity of representative compounds in the above-described assays.

TABLE 3
Activity of Representative Compounds

| COMPOUND | IC₅₀ (μM) |
|---|---|
| 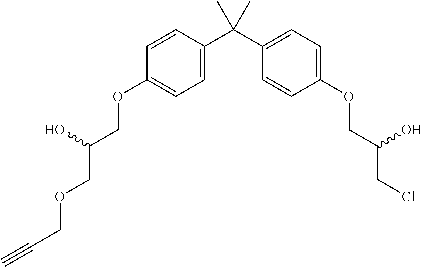 [SMILES: C#CCOCC(O)COc1ccc(C(C)(C)c2ccc(OCC(O)CCl)cc2)cc1] 3 | 1.6 |
| 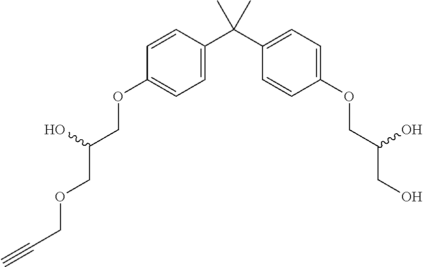 [SMILES: C#CCOCC(O)COc1ccc(C(C)(C)c2ccc(OCC(O)CO)cc2)cc1] 5 | 4.5 |
| 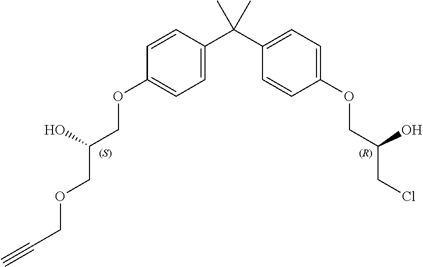 10 | 2.2 |
| 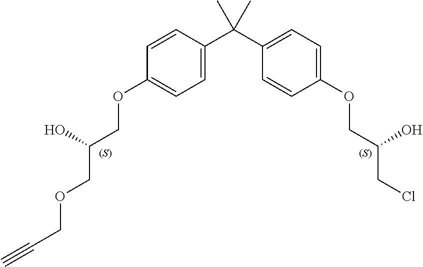 12 | 2.2 |
| 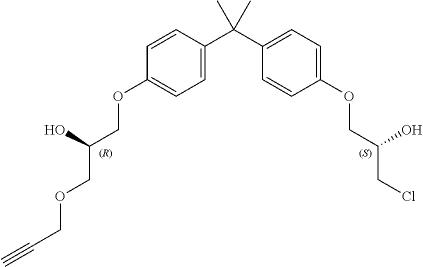 16 | 6.94 |
| 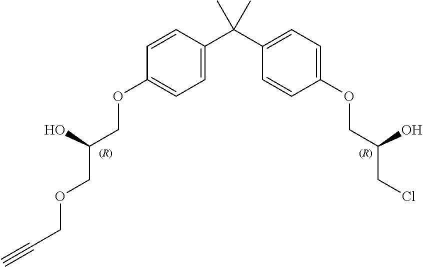 18 | 1.3 |
| 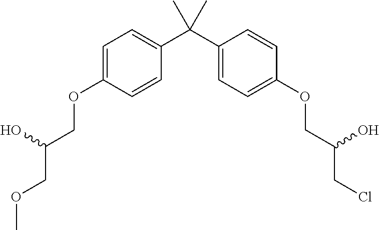 [SMILES: COCC(O)COc1ccc(C(C)(C)c2ccc(OCC(O)CCl)cc2)cc1] 30 | 11.47 |
| 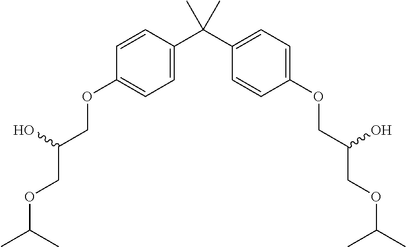 [SMILES: CC(C)OCC(O)COc1ccc(C(C)(C)c2ccc(OCC(O)COC(C)C)cc2)cc1] 31 | 4.5 |
| 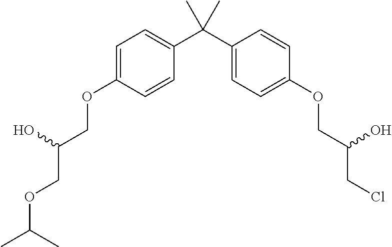 [SMILES: CC(C)OCC(O)COc1ccc(C(C)(C)c2ccc(OCC(O)CCl)cc2)cc1] 32 | 4.5 |
| 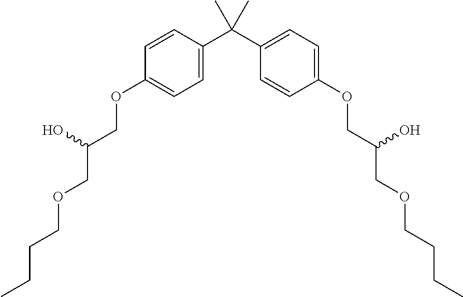 [SMILES: CCCCOCC(O)COc1ccc(C(C)(C)c2ccc(OCC(O)COCCCC)cc2)cc1] 33 | 0.70 |
| 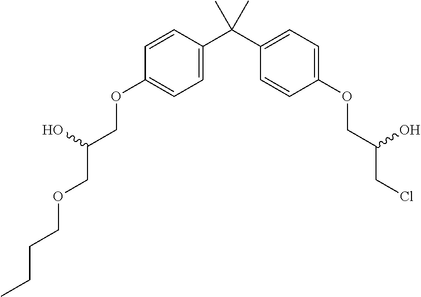 [SMILES: CCCCOCC(O)COc1ccc(C(C)(C)c2ccc(OCC(O)CCl)cc2)cc1] 34 | 1.74 |

### Example 31

### In Vivo and In Vitro Activity of Representative Compounds

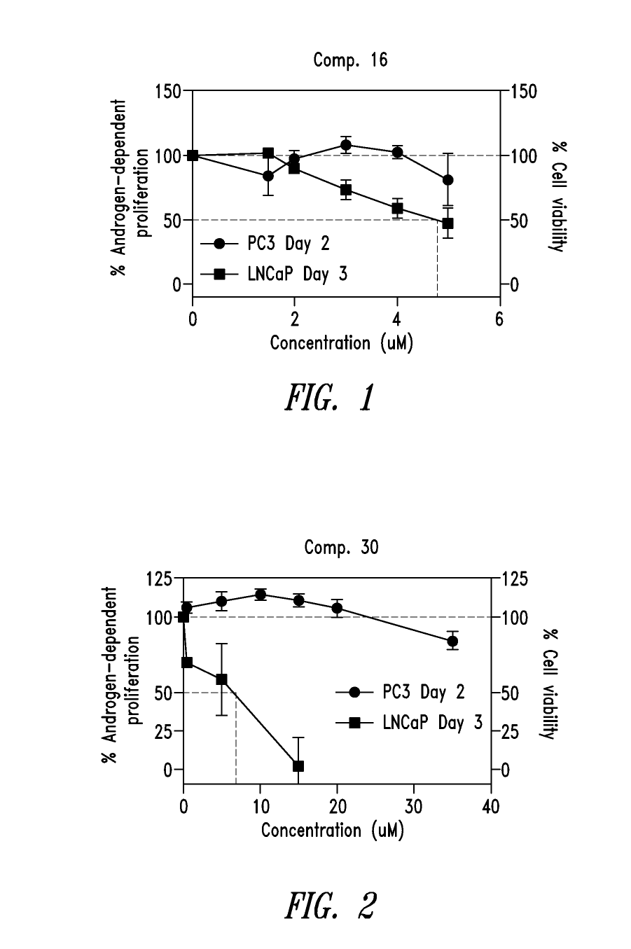

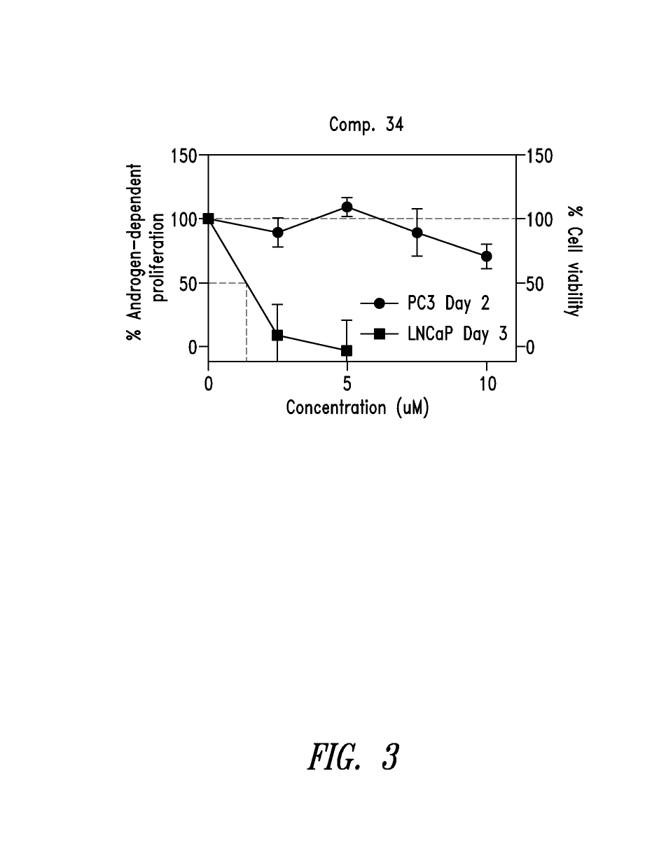

In vitro activity of representative compounds was determined according to the following procedure. LNCaP human prostate cancer cells were maintained in phenol red-free RPMI 1640 medium with 0.5% (v/v) fetal bovine serum, while PC3 human prostate cancer cells were cultured in phenol red DMEM medium with 0.5% (v/v) fetal bovine serum at 37° C. Cells were seeded in 96-well plates for 24 hrs before pre-treatment for 1 hour with representative compounds of the invention before treatment of LNCaP cells with 0.1 nM R1881 (a synthetic androgen). LNCaP cells were incubated for 3 days with R1881, while the duration of the experiment was 2 days for PC3 cells in the absence of R1881. AlamarBlue reagent (Invitrogen) was added to the cells prior to incubation for an additional 2 hrs. Fluorescence was measured at 570 nm via Safire 2 Fluorescence/Luminescence Reader (Tecan). LNCaP and PC3 data for compounds 16, 30 and 34 are presented in FIGS. 1, 2 and 3, respectively.

In vivo dose response of representative compounds and comparative compound A (i.e., (R)-3-(4-(2-(4-((S)-3-chloro-2-hydroxypropoxy)phenyl)prop an-2-yl)phenoxy)propane-1,2-diol, structure below) was determined according to the following procedure:

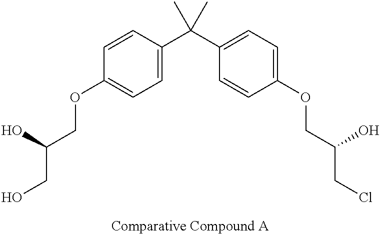 [SMILES: CC(C)(c1ccc(OC[C@H](O)CO)cc1)c1ccc(OC[C@H](O)CCl)cc1]

Male athymic SCID-NOD mice, 6- to 8-weeks old, were inoculated subcutaneously with LNCaP cells (1×10⁶) suspended in 75 μl of RPMI 1640 (5% FBS) and 75 μl of Matrigel (Becton Dickinson Labware) in the flank region via a 27-gauge needle under isofluorane anesthesia. Mice bearing LNCaP subcutaneous tumors were castrated when tumor volumes were approximately 100 mm³. Seven days after castration, mice were injected intravenously by tail vein every other day for a total of 7 doses with representative compounds of the invention in 15% DMSO and 25.5% PEG. The experiment was completed 2 days after the last injection. Tumours were measured with calipers and their volumes calculated by the formula L×W×H×0.5236.

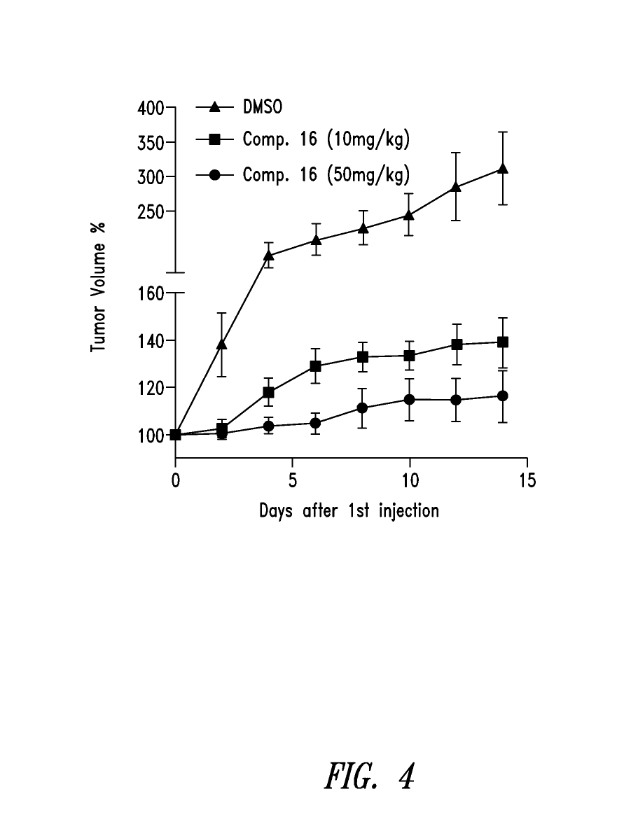

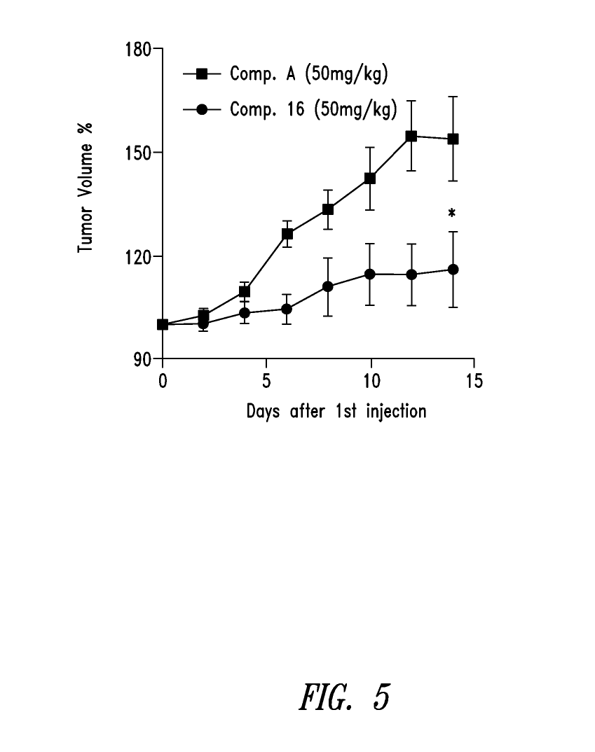

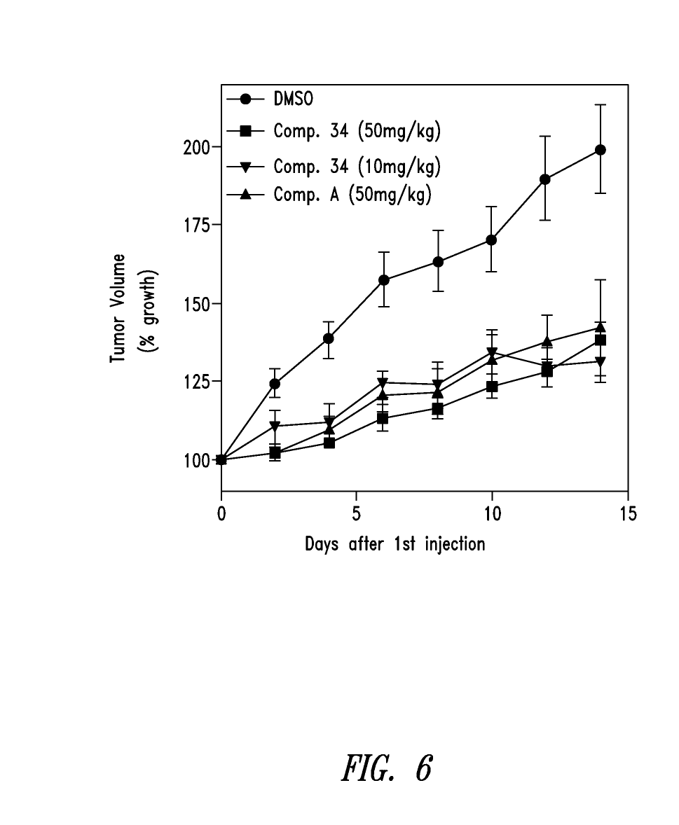

FIG. 4 shows the dose response of compound 16 at 10 mg/kg and 50 mg/kg. FIGS. 5 and 6 show dose response of Compounds 16 and 34, respectively, compared to Comparative Compound A. The data indicate that the compounds of the invention inhibit tumor growth in a dose dependent manner at levels greater than both control (DMSO) and Comparative Compound A.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A compound having a structure of Formula I: 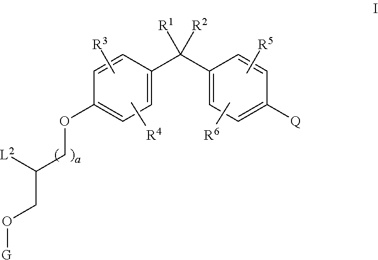
or a pharmaceutically acceptable salt thereof, wherein:
G is a linear, branched or non-aromatic cyclic, substituted or unsubstituted, saturated or unsaturated C₁-C₂₀ alkyl, wherein one or more carbon atoms of the C₁-C₂₀ alkyl may optionally be replaced with an oxygen atom;
a is 0 or 1;
R¹ and R² are each independently H or linear or branched, substituted or unsubstituted, saturated or unsaturated C₁-C₁₀ alkyl, or R¹ and R² together may form a substituted or unsubstituted, saturated or unsaturated cyclic C₃-C₁₀ alkyl;
R³, R⁴, R⁵ and R⁶ are each independently H, halo or linear or branched, substituted or unsubstituted, saturated or unsaturated C₁-C₁₀ alkyl; 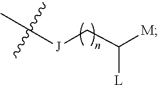
Q is
J is G¹, O, CH₂, CHG¹, CG¹₂, S, NH, NG¹, SO, SO₂, or NR;
M is H, OH, F, Cl, Br, CH₂OH, CH₂F, CH₂Cl, CHCl₂, CCl₃, CH₂Br, CHBr₂ or CBr₃;
L is H, A-D or —CH₂-A-D;
A is O, S, NH, NG¹, N⁺H₂, or N⁺HG¹;
D is H, G¹, R, or a moiety from TABLE 1;
R is C₁-C₁₀ acyl;
n is 0, 1, 2, 3, 4, 5, 6, 7 or 8;
L² is H or A²-D²;
A² is O, S, SO, SO₂, NH, NG¹, N⁺H₂ or N⁺HG¹;
D² is H, G¹, R⁷, or a moiety from TABLE 1;
G¹ is a linear or branched, aromatic or non-aromatic cyclic, substituted or unsubstituted, saturated or unsaturated C₂-C₁₀ alkyl, and
wherein the optional substituents for any of the C₁-C₂₀ alkyl, C₁-C₁₀ alkyl and cyclic C₃-C₁₀ alkyl moieties are each independently oxo, OR⁸, COOH, R⁹, OH, OR⁹, F, Cl, Br, I, NH₂, NHR⁹, N(R⁹)₂, CN, SH, SR⁹, SO₃H, SO₃R⁹, SO₂R⁹, OSO₃R⁹, OR, CO₂R⁹, CONH₂, CONHR⁹, CONHR, CON(R⁹)₂, NHR, OPO₃H₃, CONR⁹R, NR⁹R or NO₂, wherein R⁸ is a moiety from TABLE 1 and each R⁹ is independently unsubstituted C₁-C₁₀ alkyl, wherein the use is for modulating androgen receptor (AR) activity;
provided that M is not CH₂Cl when G is isopropyl and L is not H when G is a saturated C₁-C₂₀ alkyl, wherein one or more carbon atoms of the saturated C₁-C₂₀ alkyl have been replaced with an oxygen atom.

2. The compound of claim 1, wherein the compound has one of the following Formulas Ia or Ib:

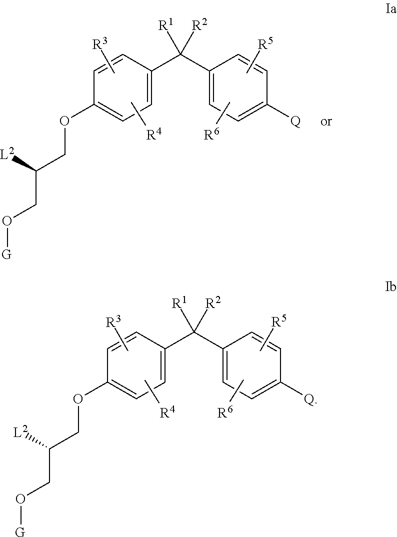

3. The compound of claim 1, wherein M is H, F, Cl, Br, CH₂OH, CH₂F, CH₂Cl, CHCl₂, CCl₃, CH₂Br, CHBr₂ or CBr₃ and L is H or A-D.

4. The compound of claim 1, wherein G is methyl, ethyl or a C₄-C₂₀ alkyl.

5. The compound of claim 1, wherein M is H, OH, F, Cl, Br, CH₂OH, CH₂F, CCl₃, CH₂Br, CHBr₂ or CBr₃.

6. The compound of claim 1, wherein M is H, OH, CH₂OH, CCl₃, CHBr₂ or CBr₃.

7. The compound of claim 1, wherein L is A-D or —CH₂-A-D.

8. The compound of claim 1, wherein G is a linear, branched or non-aromatic cyclic, substituted or unsubstituted, unsaturated C₁-C₂₀ alkyl, wherein one or more carbon atoms of the unsaturated C₁-C₂₀ alkyl may optionally be replaced with an oxygen atom.

9. The compound of claim 1, wherein G is a linear, branched or non-aromatic cyclic, substituted or unsubstituted, saturated C₁-C₂₀ alkyl, wherein one or more carbon atoms of the C₁-C₂₀ alkyl may optionally be replaced with an oxygen atom.

10. The compound of claim 9, wherein none of the carbon atoms of the C₁-C₂₀ alkyl are replaced with an oxygen atom.

11. The compound of claim 1, wherein the compound has one of the following Formulas II or III: 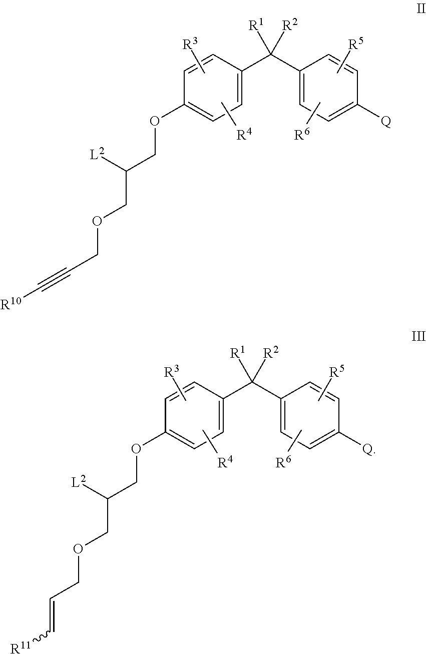
wherein R¹⁰ and R¹¹ are each independently H or linear or branched, substituted or unsubstituted, saturated or unsaturated C₁-C₆ alkyl.

12. The compound of claim 11, wherein the compound has one of the following Formulas IIa, IIb, IIIa or IIIb:

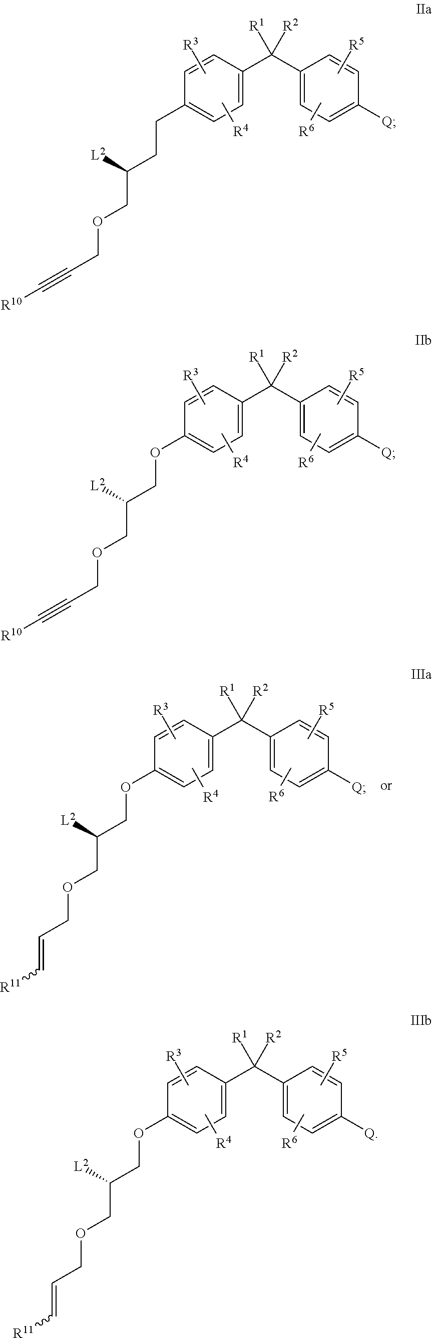

13. The compound of claim 1, wherein the compound has one of the following Formulas IV or V:

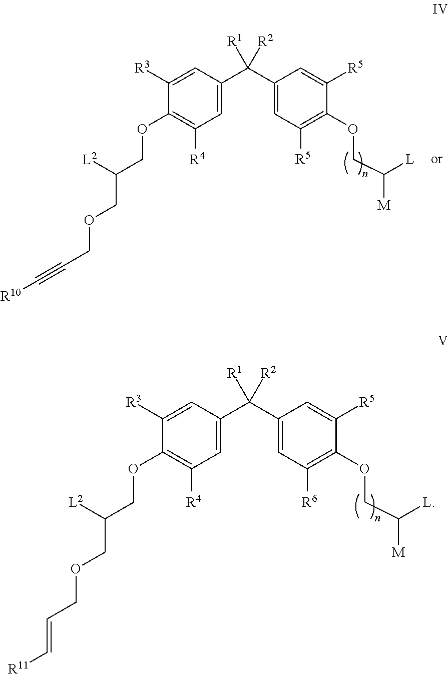

14. The compound of claim 13, wherein the compound has one of the following structures IVa, IVb, IVc, IVd, Va, Vb, Vc or Vd:

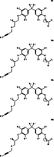

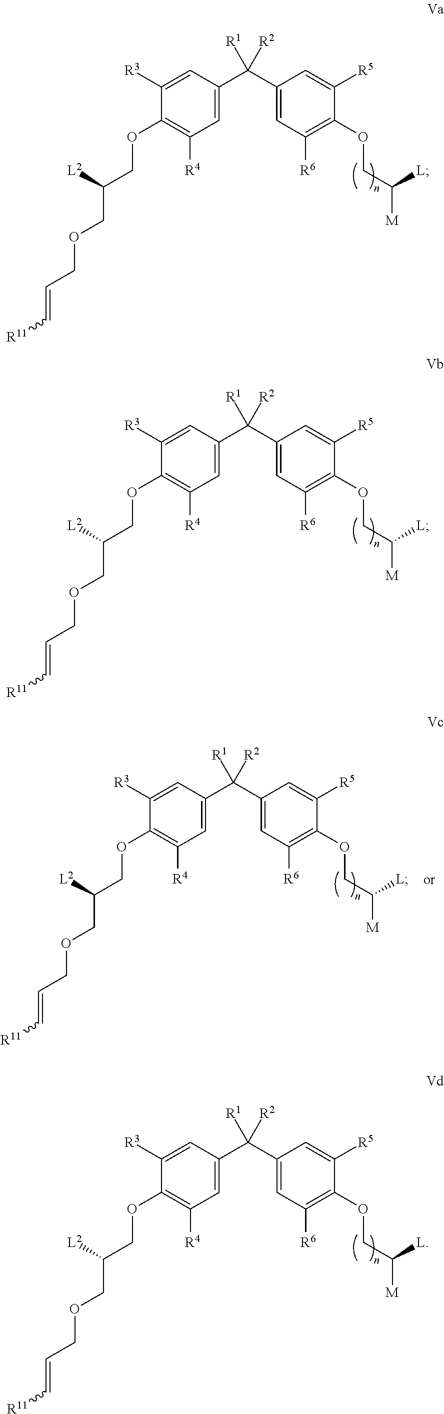

15. The compound of claim 1, wherein the compound has one of the following Formulas VI or VII: 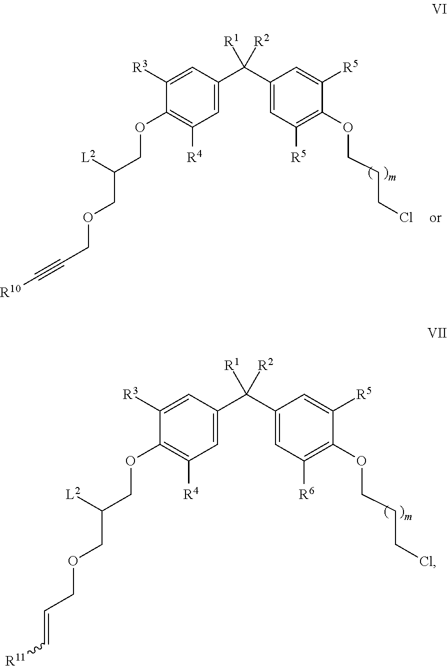
wherein m is 0, 1, 2, 3, 4 or 5.

16. The compound of claim 15, wherein the compound has one of the following Formulas VIIa, VIIb, VIIa or VIIb:

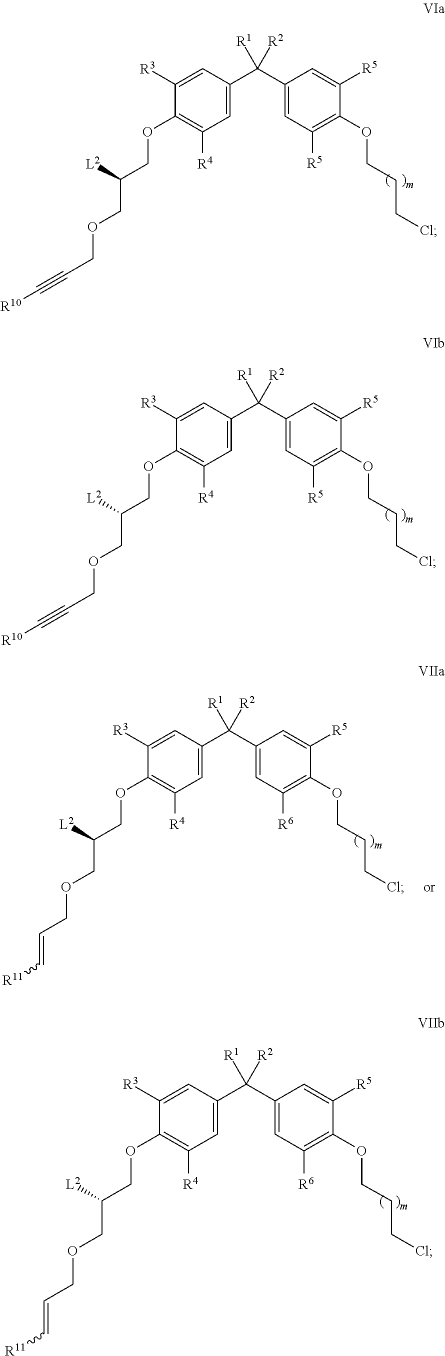

17. The compound of claim 1, wherein the compound has one of the following Formulas VIII, IX or XI:

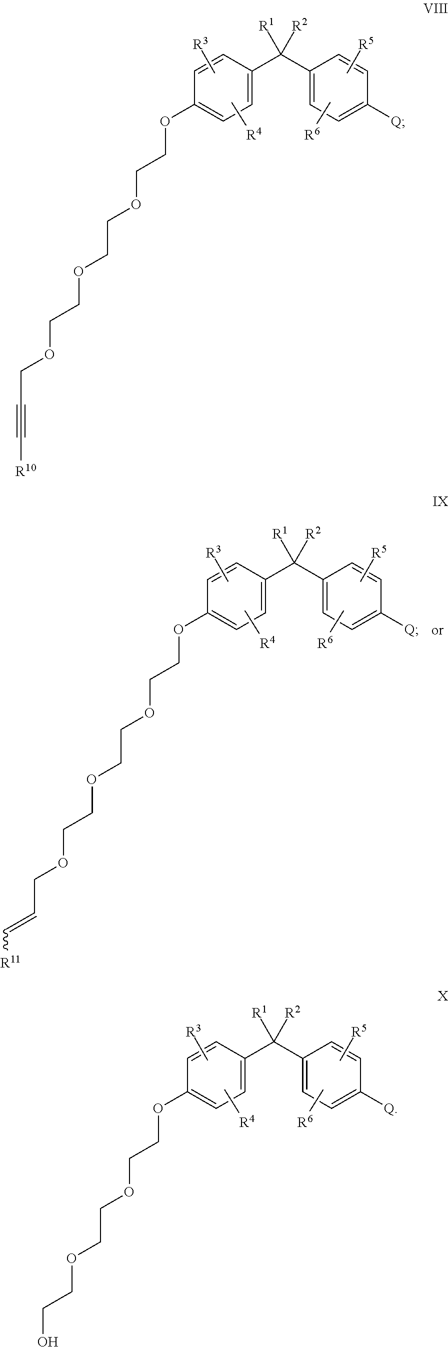

18. The compound of claim 17, wherein the compound has one of the following Formulas XI, XII, or XIII:

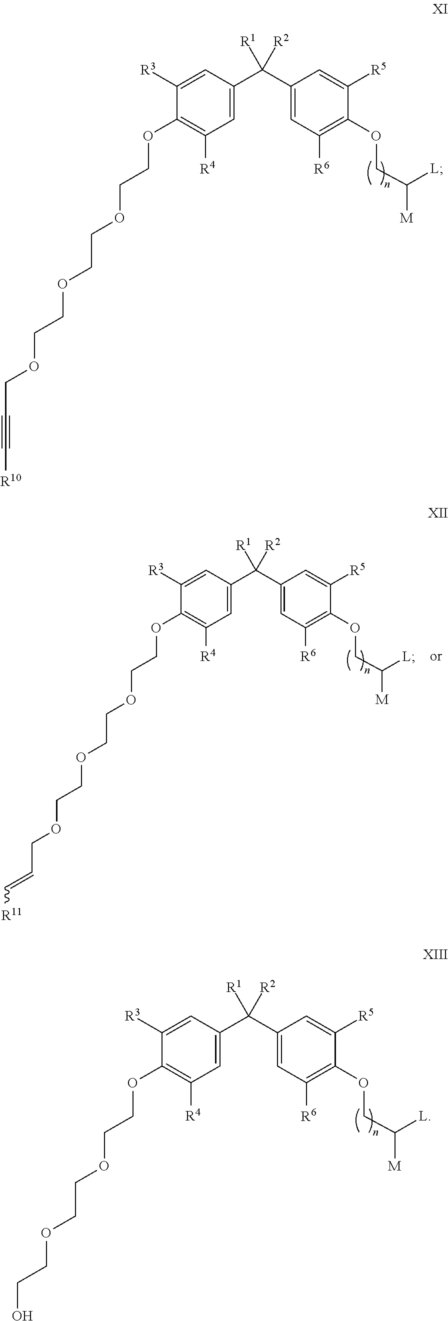

19. The compound of claim 18, wherein the compound has one of the following Formulas XIa, XIb, XIIa, XIIb, XIIIa or XIIIb:

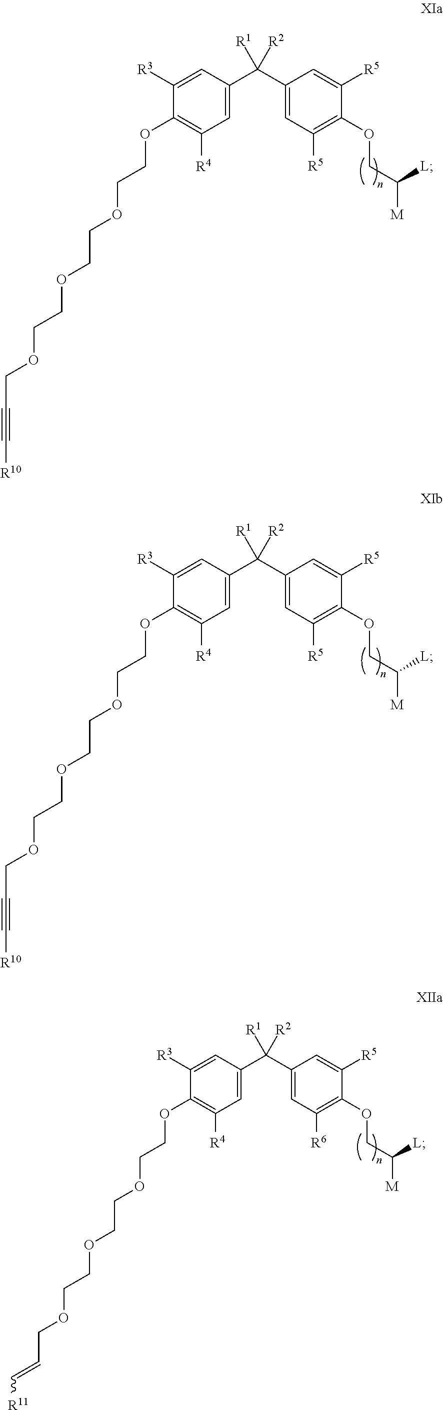

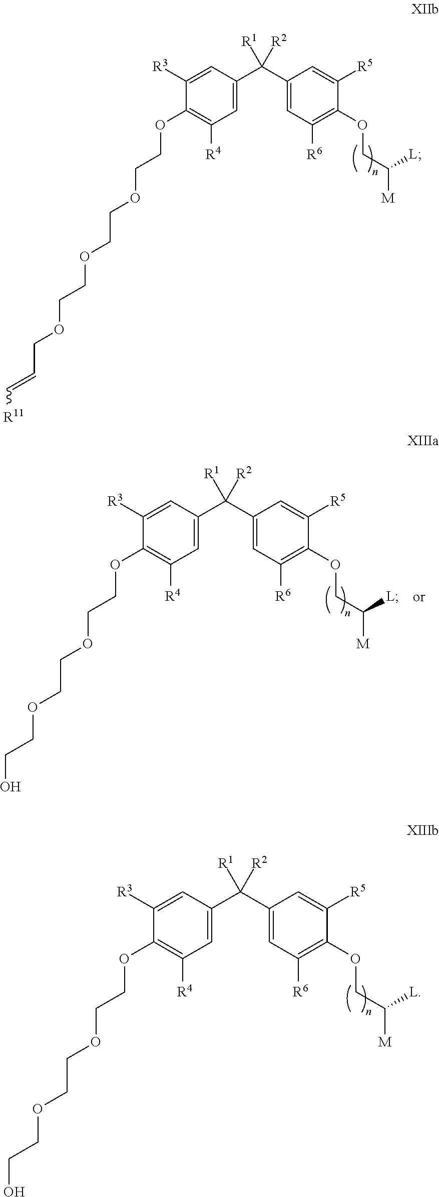

20. The compound of any claim 1, wherein the compound has one of the following Formulas XIV, XV or XVI: 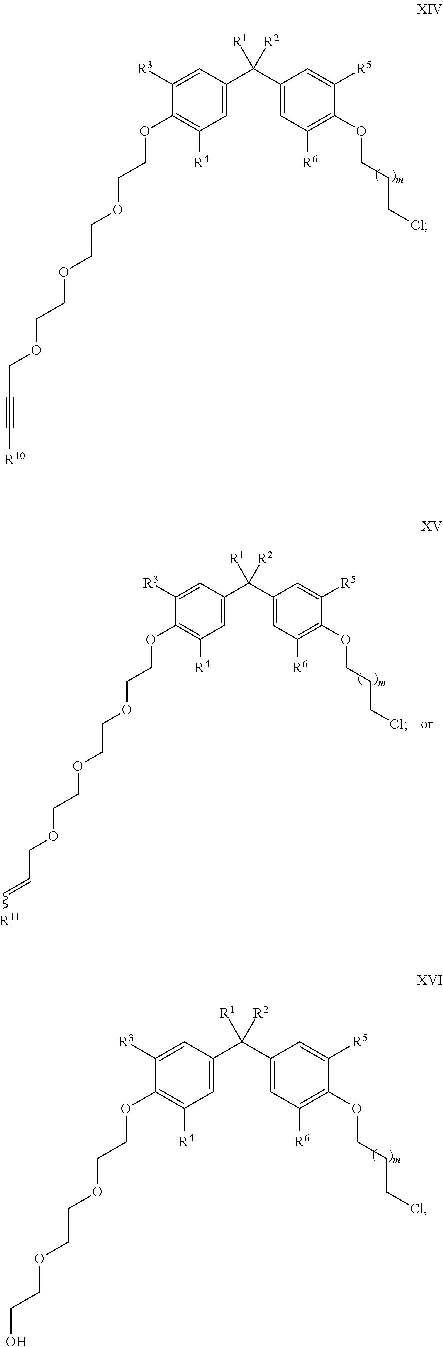
wherein m is 0, 1, 2, 3, 4 or 5.

21. The compound of claim 1, wherein the compound has one of the following Formulas XVII, XVIII or XIX:

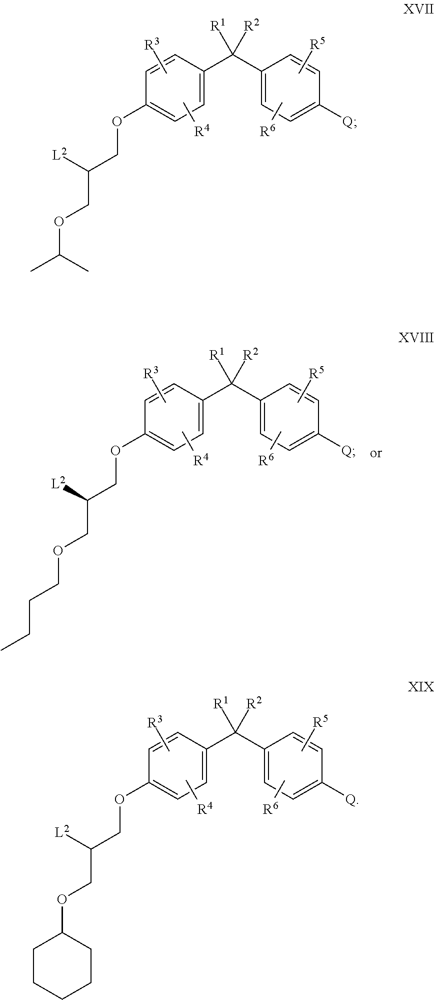

22. The compound of claim 21, wherein the compound has one of the following Formulas XVIIa, XVIIb, XVIIIa, XVIIIb, XIXa or XIXb:

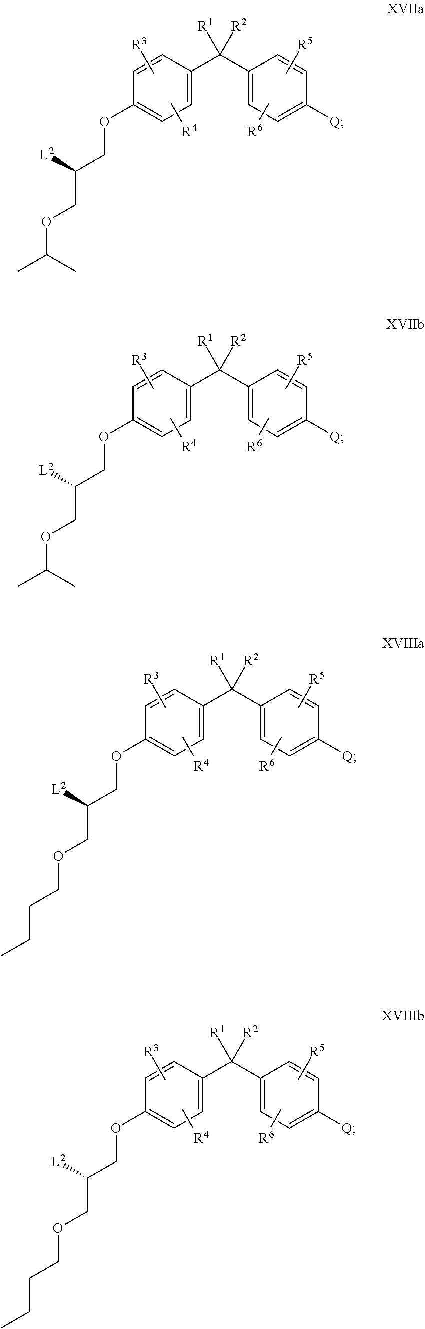

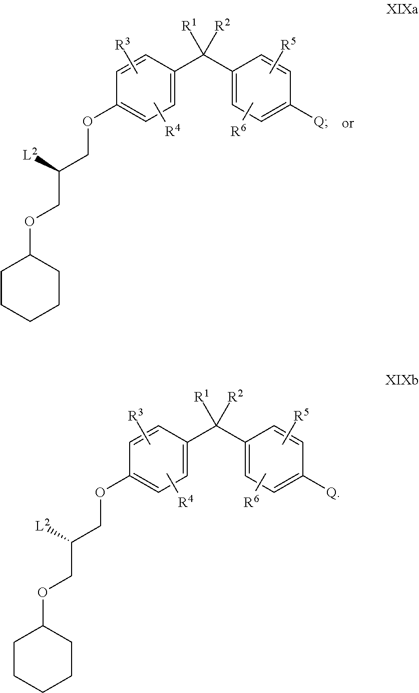

23. The compound of claim 22, wherein the compound has one of the following Formulas XX, XXI or XXII:

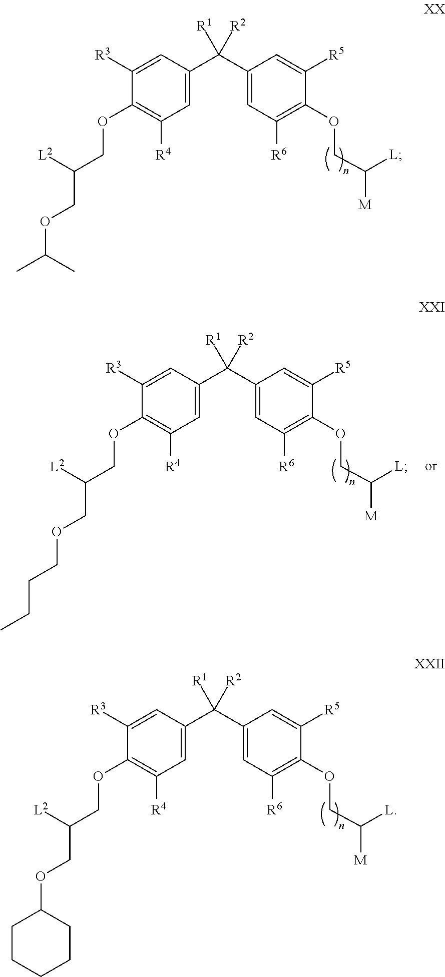

24. The compound of claim 23, wherein the compound has one of the following Formulas XXa, XXb, XXc, XXd, XXIa, XXIb, XXIc, XXId, XXIIa, XXIIb, XXIIc or XXIId:

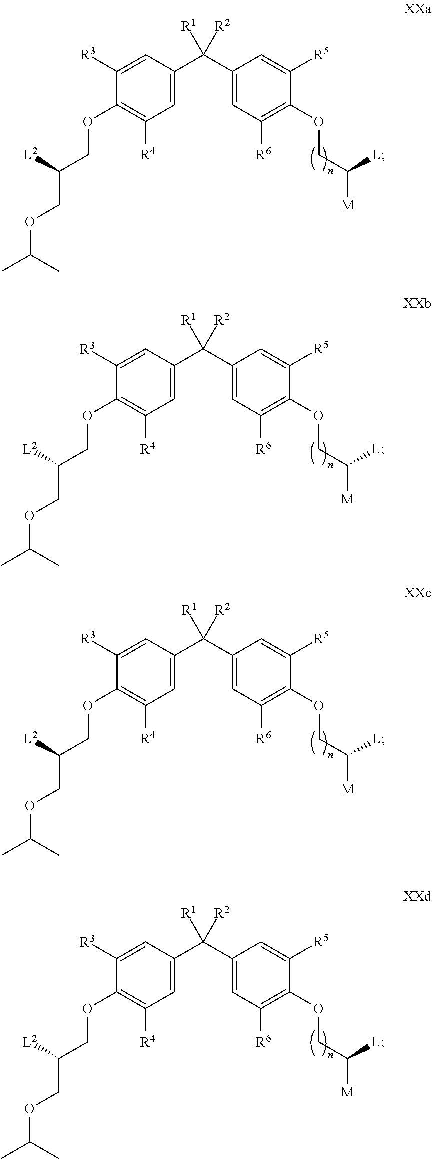

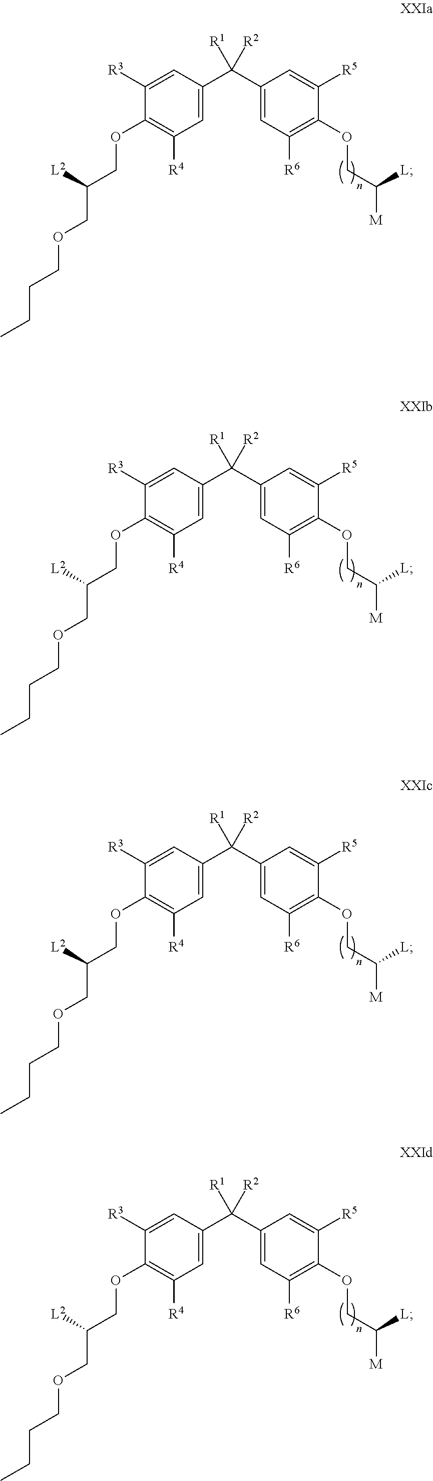

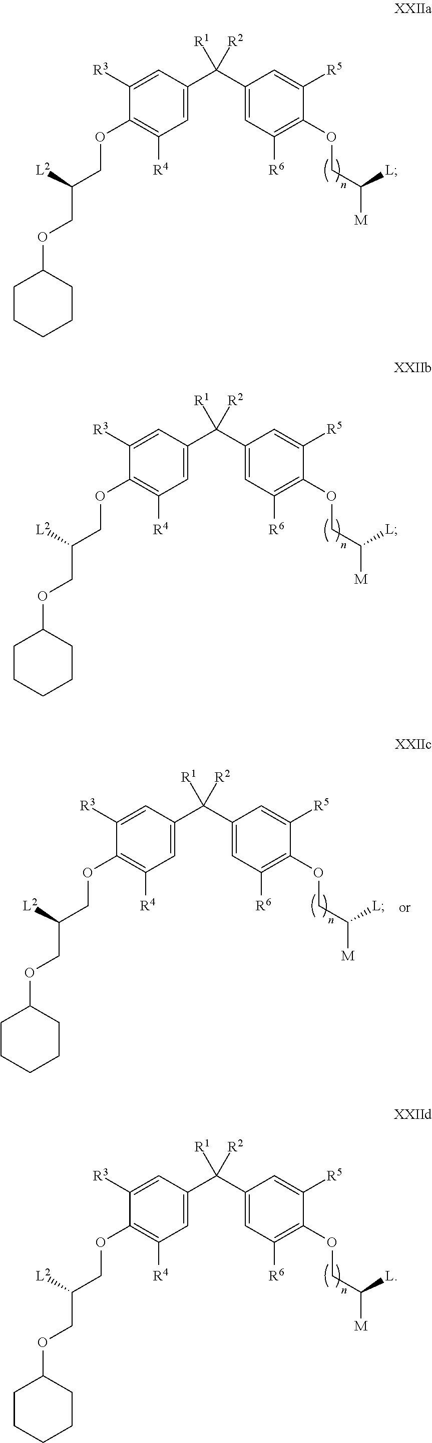

25. The compound of claim 1, wherein the compound has one of the following Formulas XXIII, XXIV or XXV:

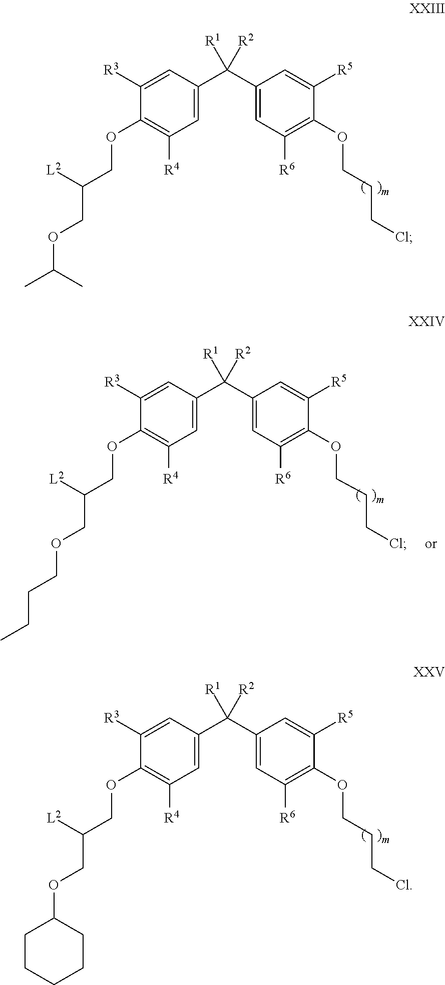

26. The compound of claim 25, wherein the compound has one of the following Formulas XXIIIa, XXIIIb, XXIVa, XXIVb, XXVa or XXVb:

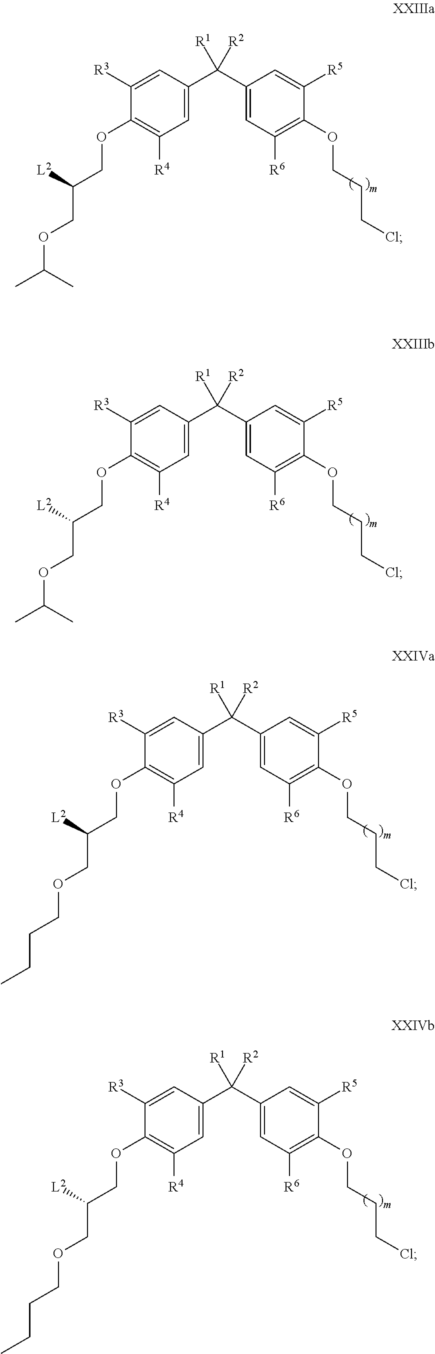

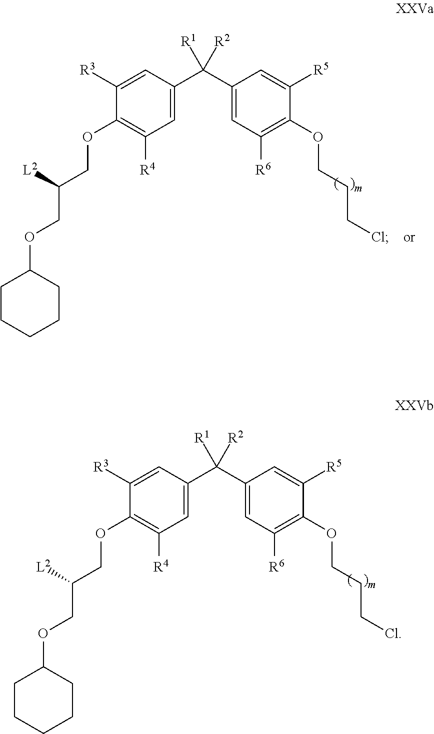

27-84. (canceled)

85. The compound claim 1, wherein Q is 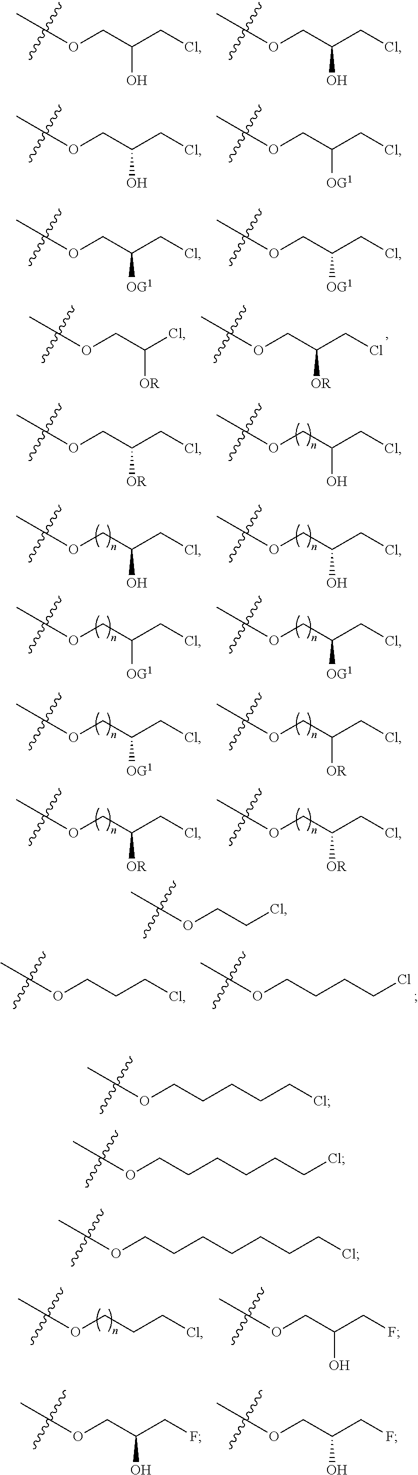 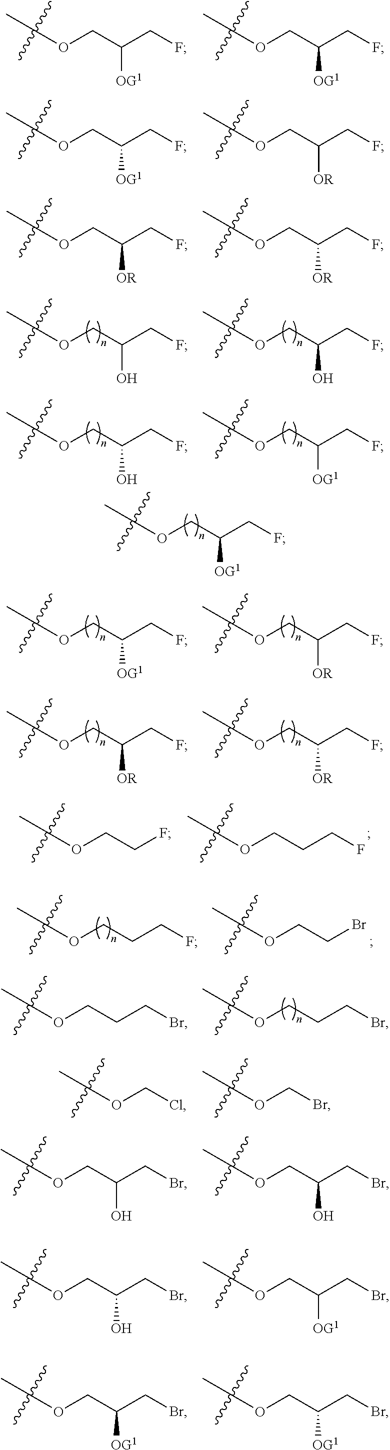 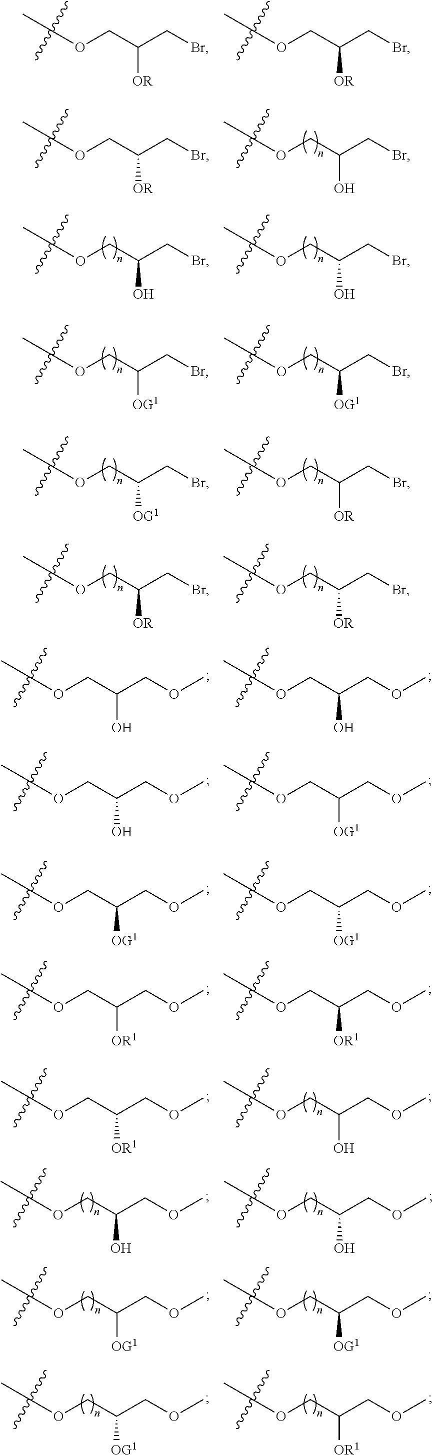 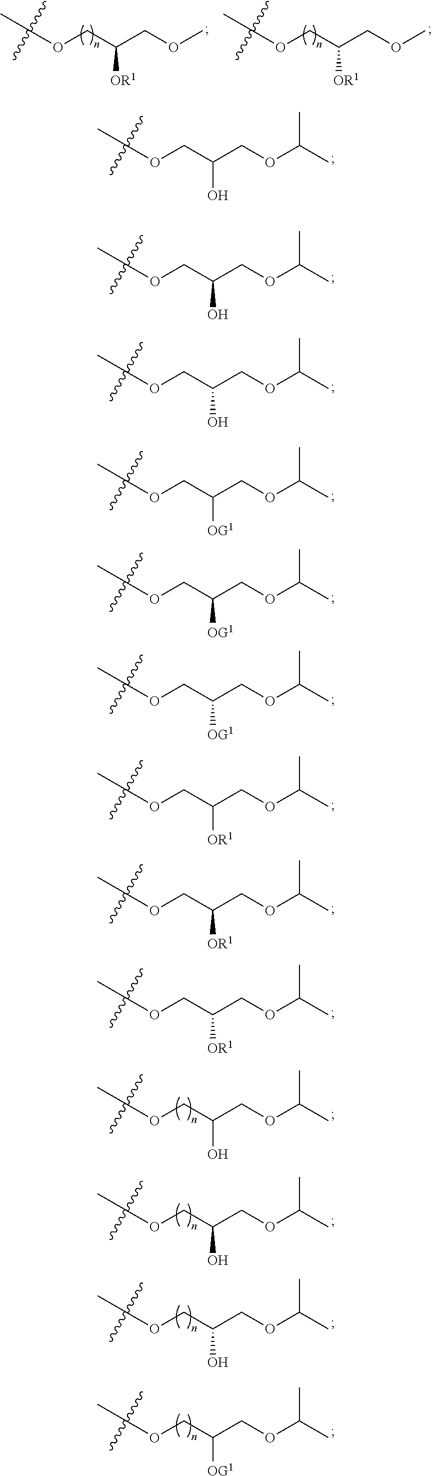 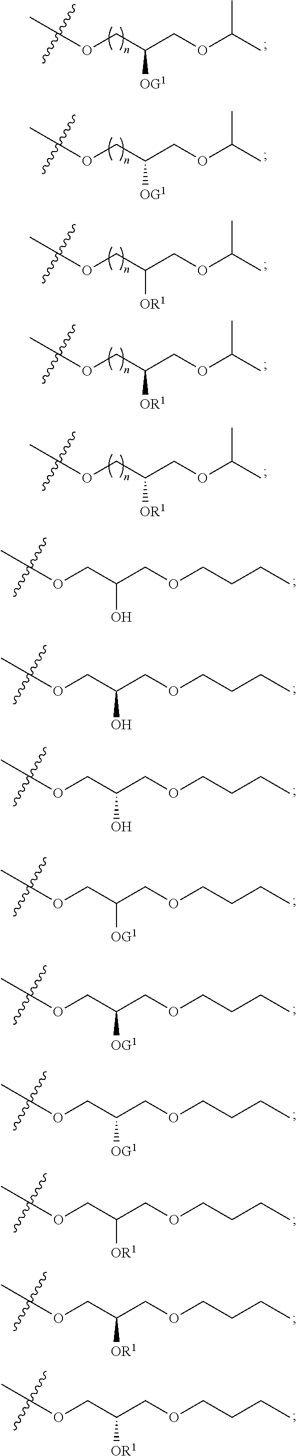 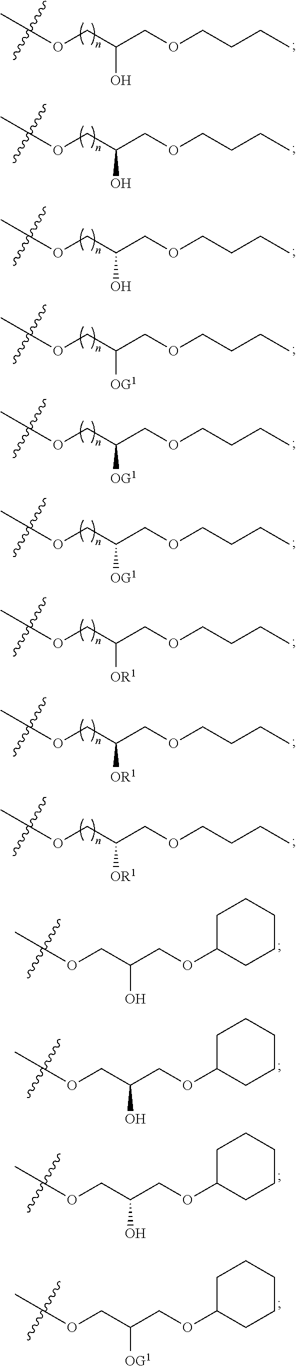 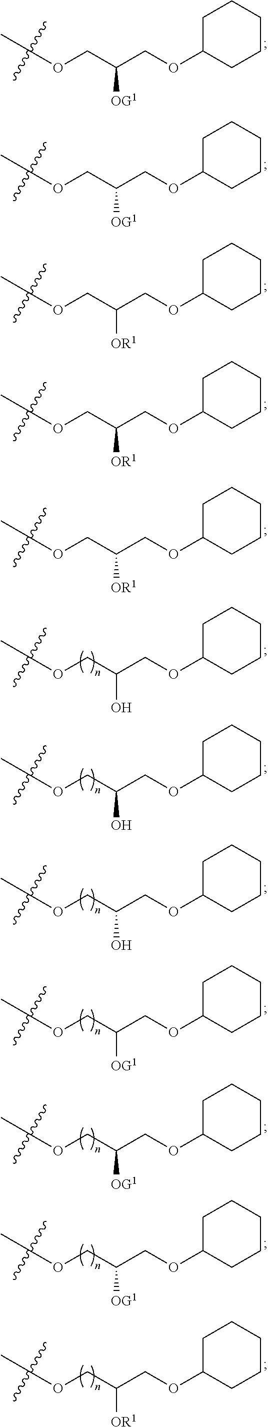 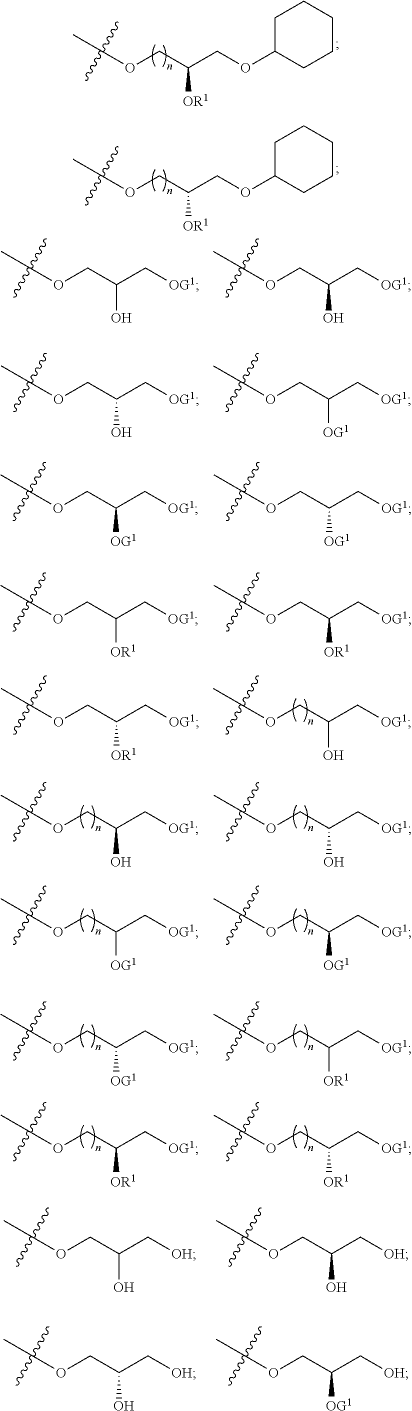 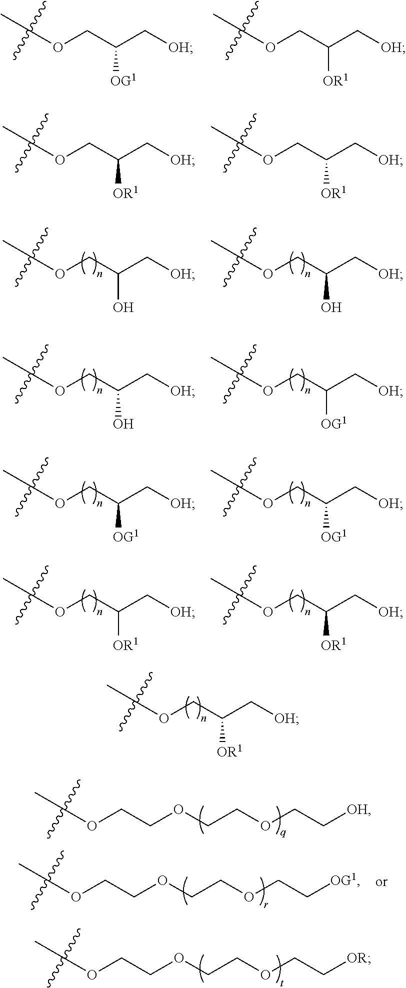
n is 0, 1, 2, 3, 4, 5, 6, 7 or 8;
each of q, r, and t is independently 0, 1, 2, 3, 4, 5, 6 or 7;
each G¹ is independently linear or branched, substituted or unsubstituted, saturated or unsaturated C₁-C₁₀ alkyl, wherein the optional substituent is oxo, OR⁸, COOH, OH, F, Cl, Br, I, NH₂, CN, SH, SO₃H, CONH₂, OPO₃H₃ or NO₂.

86. The compound of claim 1, wherein Q is 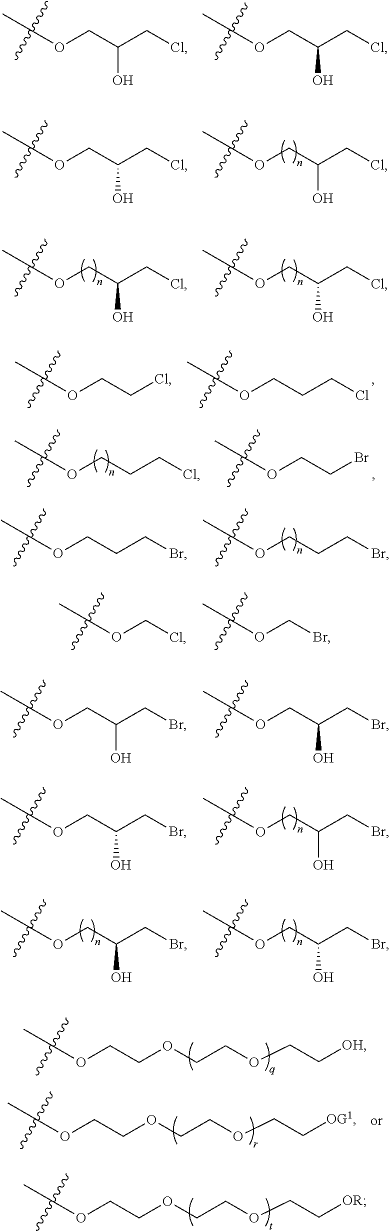
n is 0, 1, 2, 3, 4, 5, 6, 7 or 8;
each of q, r, and t is independently 0, 1, 2, 3, 4, 5, 6 or 7; and
each G¹ is independently linear or branched, substituted or unsubstituted, saturated or unsaturated C₁-C₁₀ alkyl, wherein the optional substituent is selected from the group consisting of oxo, OR⁸, COOH, OH, F, Cl, Br, I, NH₂, CN, SH, SO₃H, CONH₂, OPO₃H₃, and NO₂.

87. The compound of claim 1, wherein Q is 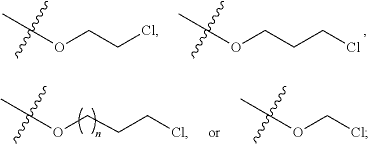
and
n is 0, 1, 2, 3, 4, 5, 6, 7 or 8.

88. The compound of claim 1, wherein Q is 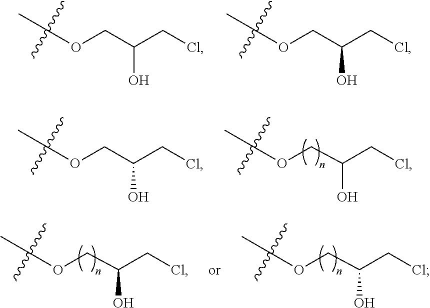
and
n is 0, 1, 2, 3, 4, 5, 6, 7 or 8.

89. The compound of claim 1, wherein Q is 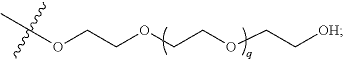
and
q is 0, 1, 2, 3, 4, 5, 6 or 7.

90. The compound of claim 1, wherein Q is

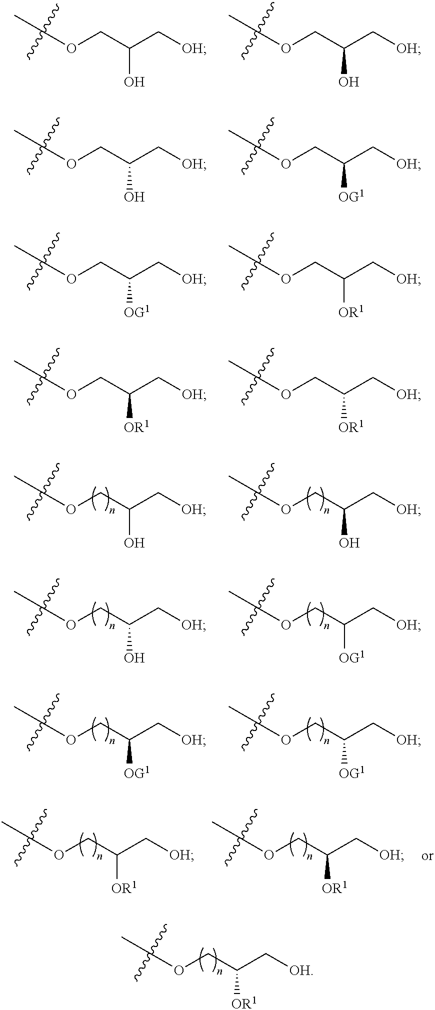

91. The compound of claim 1, wherein Q is

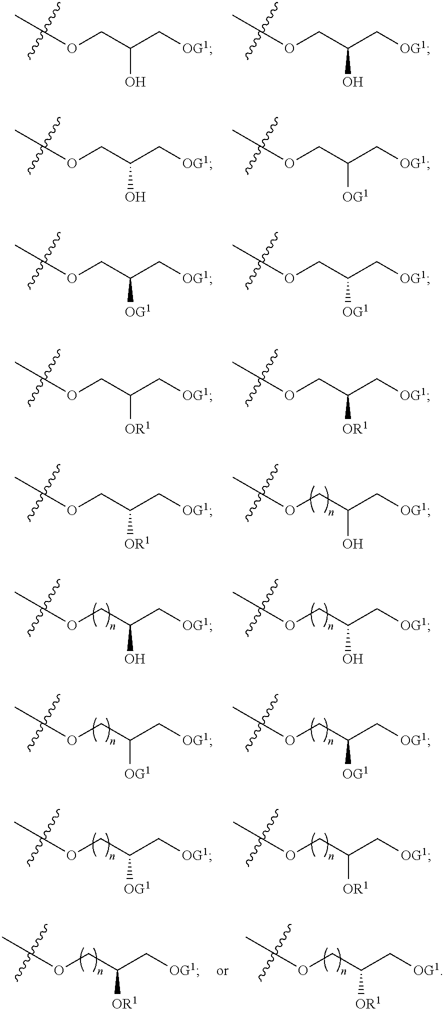

92. The compound of claim 91, wherein Q is

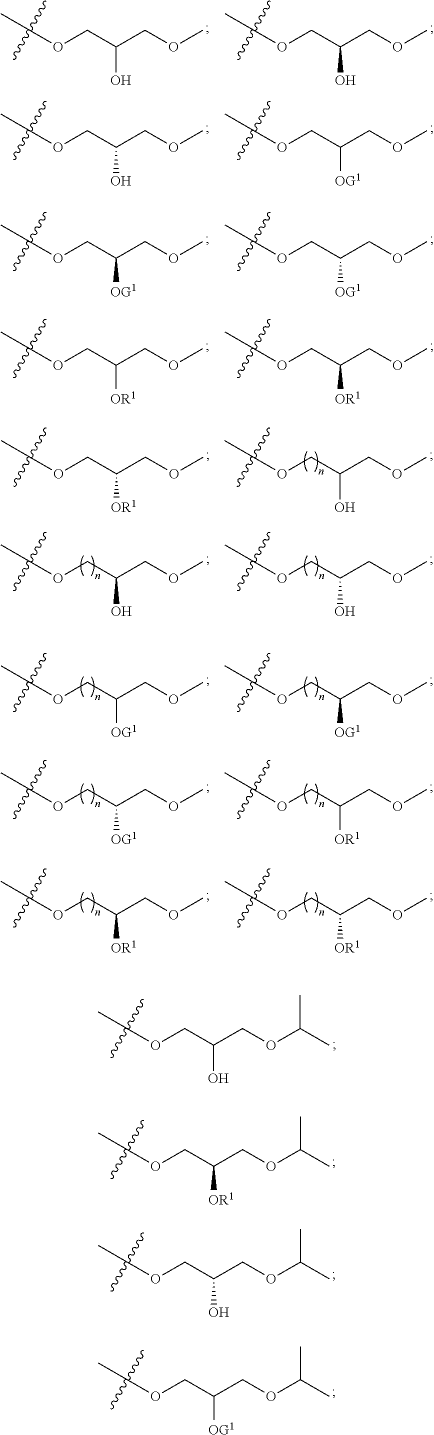

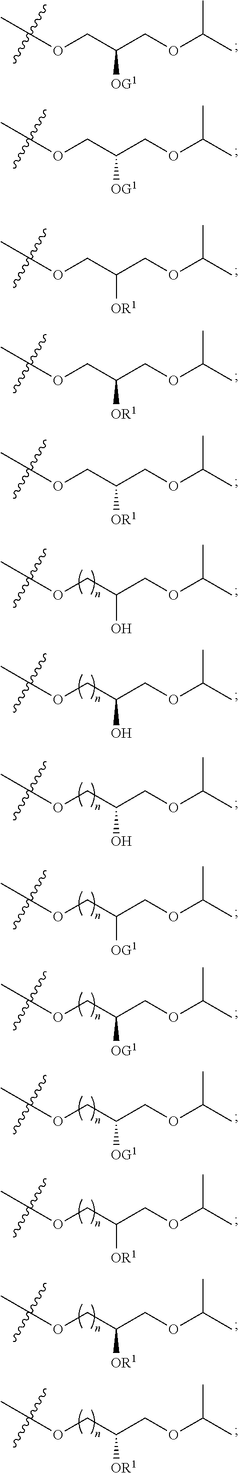

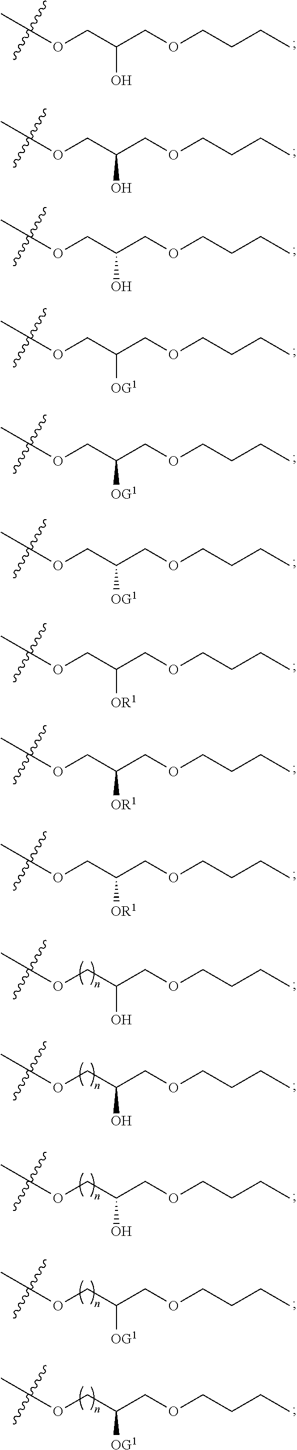

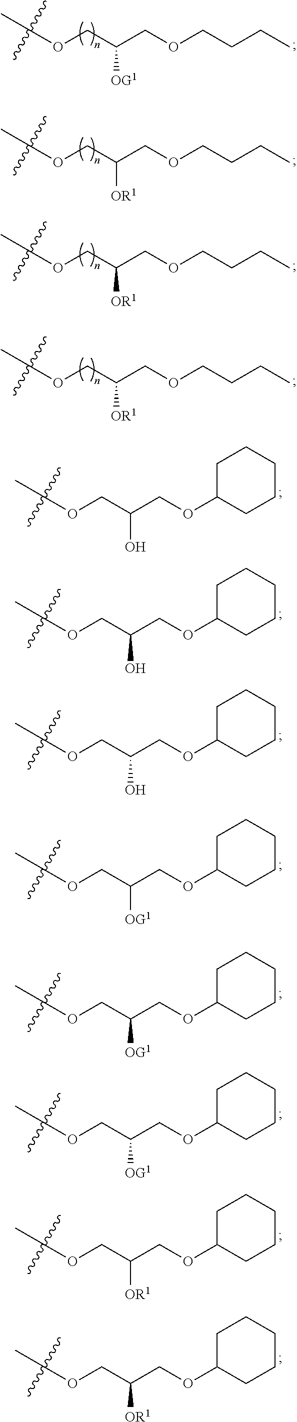

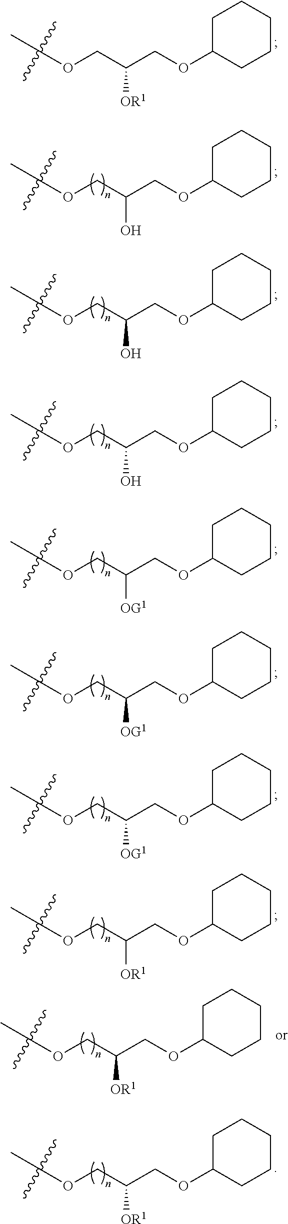

93. The compound of claim 1, wherein G, is CH₂C≡CH or CH═CH₂.

94. The compound of claim 1, wherein one or more of the OH groups of the compound is optionally substituted to replace the H with a moiety from TABLE 1.

95. The compound of claim 94, wherein the moiety from TABLE 1 is

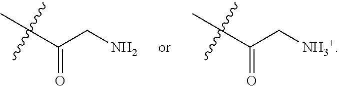

96. A compound having one of the following structures:

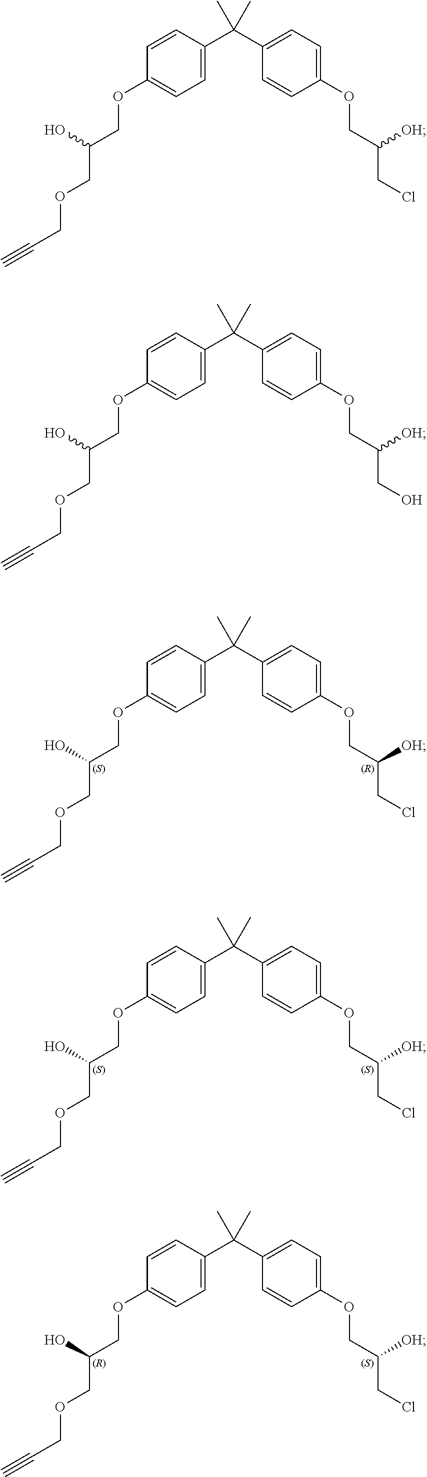

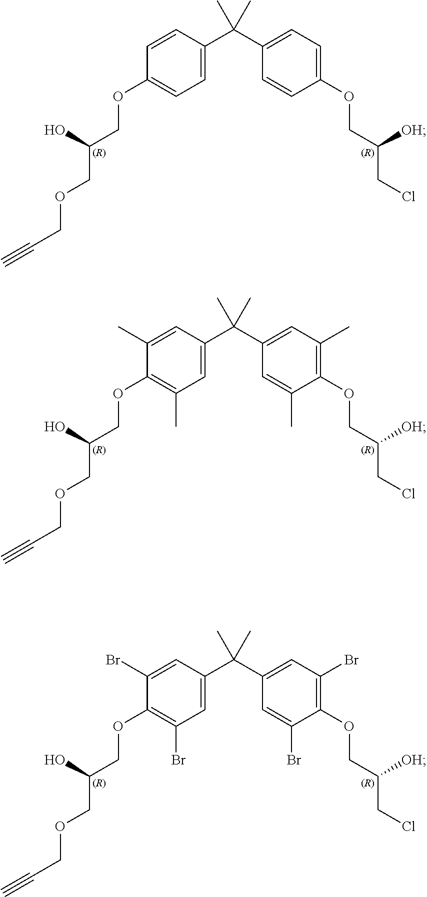

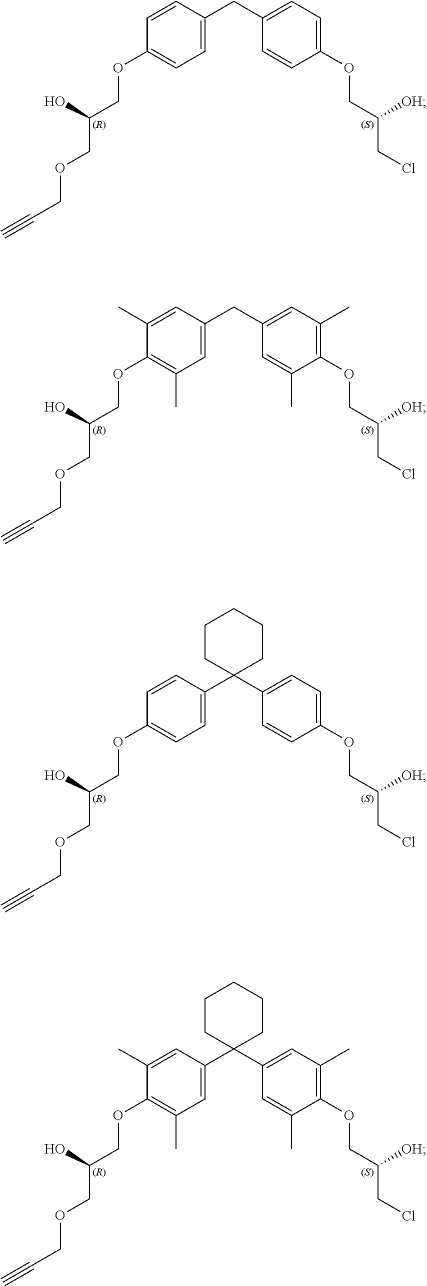

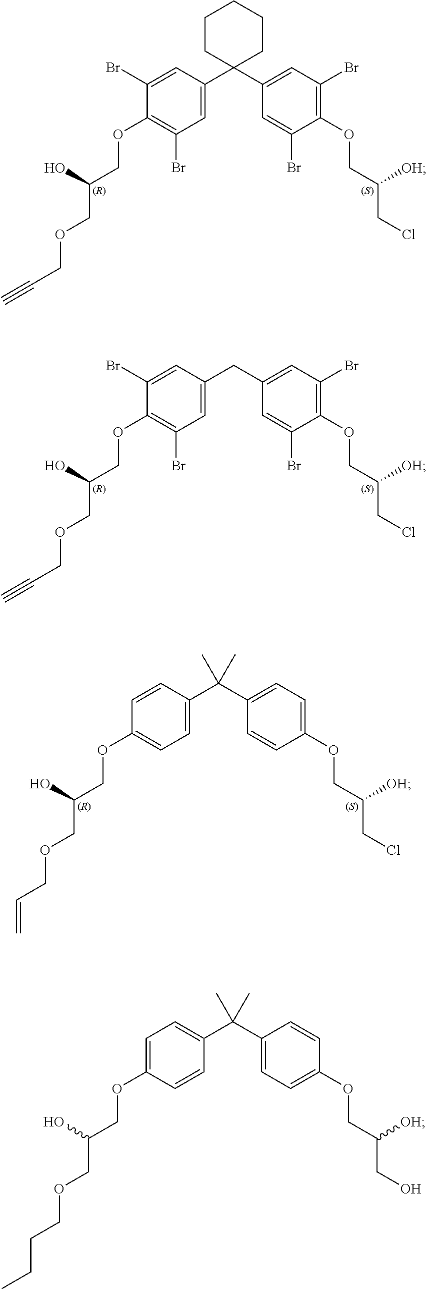

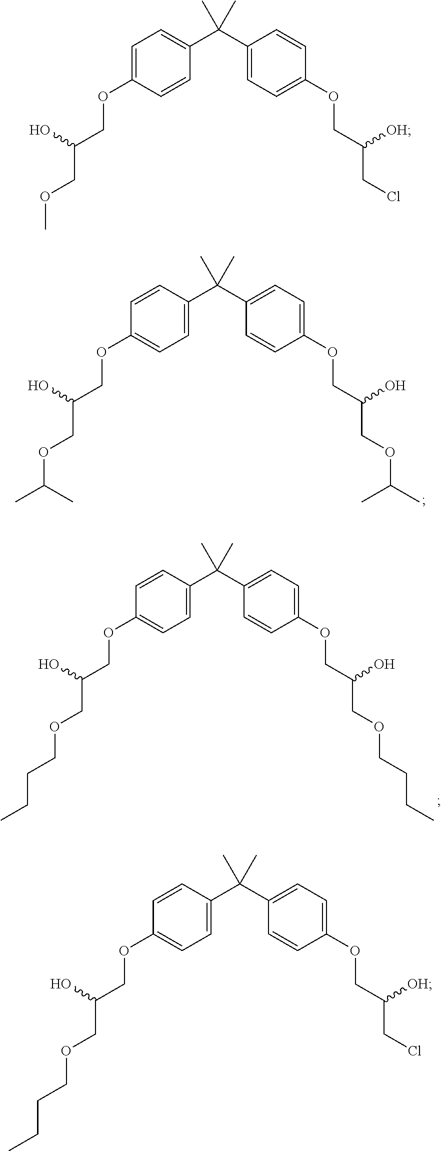

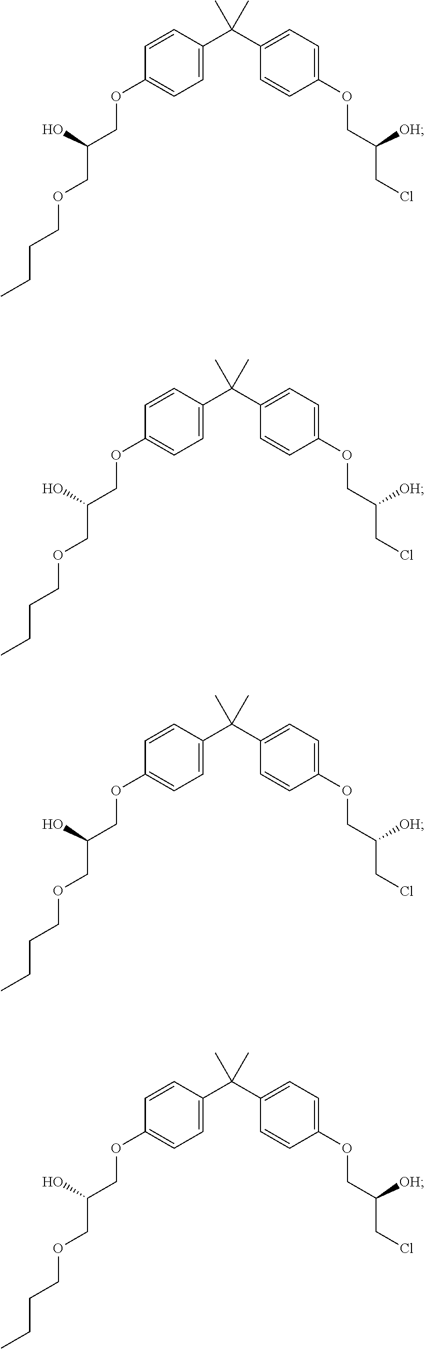 [SMILES: CCCCOC[C@@H](O)COc1ccc(C(C)(C)c2ccc(OC[C@@H](O)CCl)cc2)cc1 ; CCCCOC[C@H](O)COc1ccc(C(C)(C)c2ccc(OC[C@H](O)CCl)cc2)cc1 ; CCCCOC[C@@H](O)COc1ccc(C(C)(C)c2ccc(OC[C@H](O)CCl)cc2)cc1 ; CCCCOC[C@H](O)COc1ccc(C(C)(C)c2ccc(OC[C@@H](O)CCl)cc2)cc1]

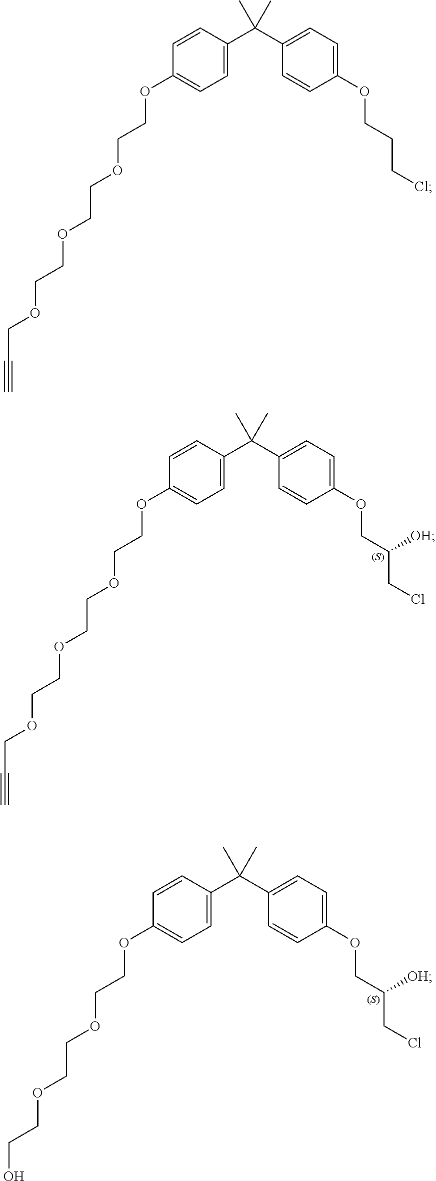

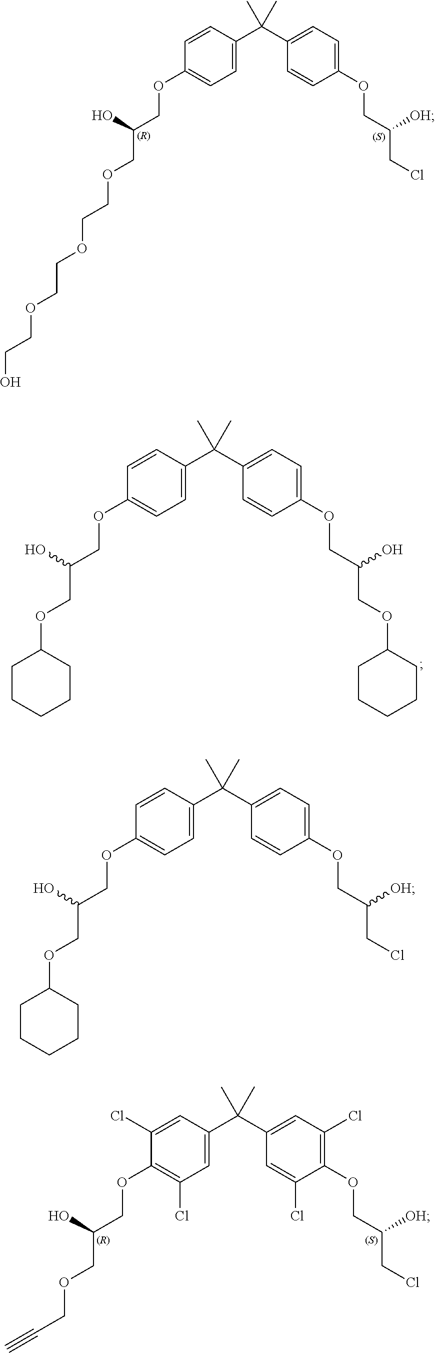

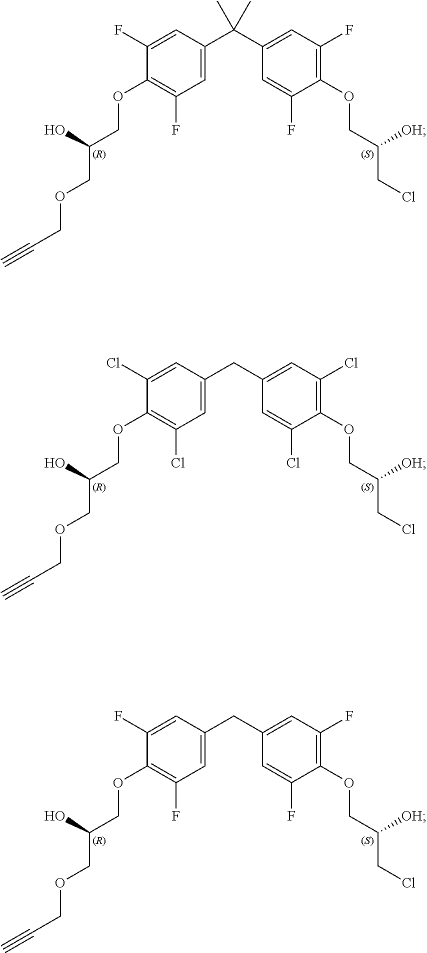

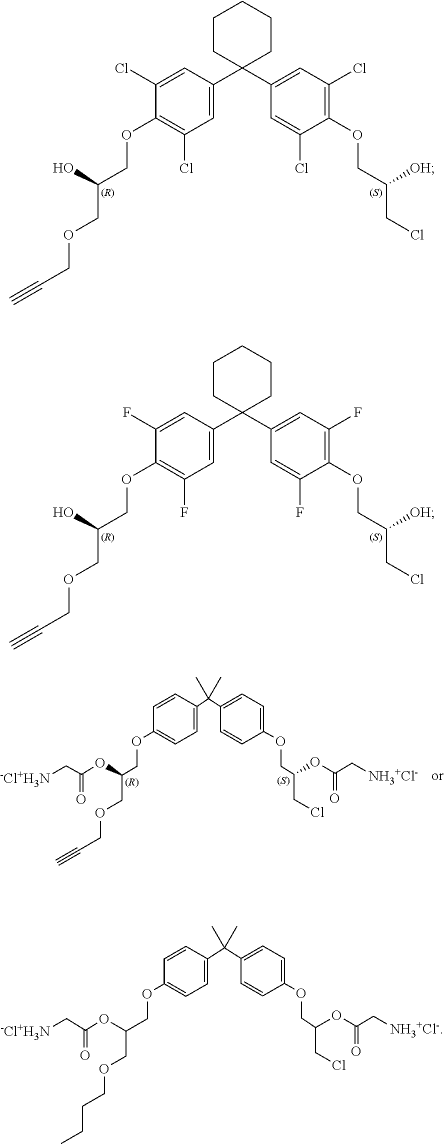

97. Use of the compound of claim 1, for modulating androgen receptor (AR) activity.

98. The use of claim 97, wherein modulating androgen receptor (AR) activity is in a mammalian cell.

99. The use of claim 97, wherein modulating androgen receptor (AR) activity is for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration.

100. The use of claim 99, wherein the indication is prostate cancer.

101. The use of claim 100, wherein the prostate cancer is castration resistant prostate cancer.

102. The use of claim 100, wherein the prostate cancer is androgen-dependent prostate cancer.

103. The use of claim 99, wherein the spinal and bulbar muscular atrophy is Kennedy's disease.

104. A method of modulating androgen receptor (AR) activity, the method comprising administering a compound of claim 1, or pharmaceutically acceptable salt thereof, to a subject in need thereof.

105. The method of claim 104, wherein modulating androgen receptor (AR) activity is for the treatment of one or more of the following: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration.

106. The method of claim 105, wherein the spinal and bulbar muscular atrophy is Kennedy's disease.

107. A pharmaceutical composition comprising a compound of claim 1 and a pharmaceutically acceptable carrier.

108. A pharmaceutical composition comprising a compound of claim 1, an additional therapeutic agent and a pharmaceutically acceptable carrier.

109. The pharmaceutical composition of claim 108, wherein the additional therapeutic agent is for treating prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy or age-related macular degeneration.

110. The pharmaceutical composition of claim 108, wherein the additional therapeutic agent is MDV3100, TOK 001, ARN-509; abiraterone, bicalutamide, nilutamide, flutamide, cyproterone acetate, docetaxel, Bevacizumab (Avastin), OSU-HDAC42, VITAXIN, sunitumib, ZD-4054, VN/124-1, Cabazitaxel (XRP-6258), MDX-010 (Ipilimumab), OGX 427, OGX 011, finasteride, dutasteride, turosteride, bexlosteride, izonsteride, FCE 28260, SKF105,111 or related compounds thereof.

111-123. (canceled)
